SEQUENCE LISTING (187 sequences)

SEQ ID NO: 1 - protein, 511 aa, synthetic construct
MALPVTALLLPLALLLHAARPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 2 - protein, 517 aa, synthetic construct
MALPVTALLLPLALLLHAARPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 3 - protein, 526 aa, synthetic construct
MALPVTALLLPLALLLHAARPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSCPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 4 - protein, 532 aa, synthetic construct
MALPVTALLLPLALLLHAARPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 5 - protein, 495 aa, synthetic construct
MALPVTALLLPLALLLHAARPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGCPYSNPSLCGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 6 - protein, 501 aa, synthetic construct
MALPVTALLLPLALLLHAARPQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGGGCPYSNPSLCGGGGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 7 - protein, 508 aa, synthetic construct
MALPVTALLLPLALLLHAARPGGGGSCPYSNPSLCGQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 8 - protein, 514 aa, synthetic construct
MALPVTALLLPLALLLHAARPGGGGSCPYSNPSLCSGGGGSGQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 9 - protein, 523 aa, synthetic construct
MALPVTALLLPLALLLHAARPGGGGSCPYSNPSLCSGGGGSCPYSNPSLCGQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 10 - protein, 529 aa, synthetic construct
MALPVTALLLPLALLLHAARPGGGGSCPYSNPSLCSGGGGSCPYSNPSLCSGGGGSGQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 11 - protein, 297 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 12 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 13 - protein, 1039 aa, synthetic construct
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERAIPIWWVLVGVLGGLLLLTILVLAMWKVGFFKRNRPPLEEDDEEGE

SEQ ID NO: 14 - protein, 272 aa, synthetic construct
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 15 - protein, 595 aa, synthetic construct
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 16 - protein, 1091 aa, synthetic construct
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQGQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPGEWLVWKQSCSSTSSTHSAAASLQCPSQVLPPASPEGETVADLQTQ

SEQ ID NO: 17 - protein, 233 aa, synthetic construct
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 18 - protein, 61 aa, synthetic construct
MKRFLFLLLTISLLVMVQIQTGLSGQNDTSQTSSPSASSNISGGIFLFFVANAIIHLFCFS

SEQ ID NO: 19 - protein, 232 aa, synthetic construct
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 20 - protein, 1676 aa, synthetic construct
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 21 - protein, 1170 aa, synthetic construct
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILACDPGLSRTCDQNTYLSGLCYLFRQNLQGPMLQGRPGFQECIKGNVDLVFLFDGSMSLQPDEFQKILDFMKDVMKKLSNTSYQFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAQGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 22 - protein, 364 aa, synthetic construct
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 23 - protein, 1032 aa, synthetic construct [sequence omitted]

SEQ ID NO: 24 - protein, 257 aa, synthetic construct
MAPAMESPTLLCVALLFFAPDGVLAVPQKPKVSLNPPWNRIFKGENVTLTCNGNNFFEVSSTKWFHNGSLSEETNSSLNIVNAKFEDSGEYKCQHQQVNESEPVYLEVFSDWLLLQASAEVVMEGQPLFLRCHGWRNWDVYKVIYYKDGEALKYWYENHNISITNATVEDSGTYYCTGKVWQLDYESEPLNITVIKAPREKYWLQFFIPLLVVILFAVDTGLFISTQQQVTFLLKIKRTRKGFRLLNPHPKPNPKNN

SEQ ID NO: 25 - protein, 574 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 26 - protein, 468 aa, synthetic construct
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 27 - protein, 1255 aa, synthetic construct [sequence omitted]

SEQ ID NO: 28 - protein, 798 aa, synthetic construct
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 29 - protein, 285 aa, synthetic construct
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 30 - protein, 269 aa, synthetic construct
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 31 - protein, 317 aa, synthetic construct
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 32 - protein, 223 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 33 - protein, 189 aa, synthetic construct
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 34 - protein, 288 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 35 - protein, 9 aa, synthetic construct
  REGION 1..9: note = mAb-specific epitope
CPYSNPSLC

SEQ ID NO: 36 - protein, 24 aa, synthetic construct
  REGION 1..24: note = mAb-specific epitope
NSELLSLINDMPITNDQKKLMSNN

SEQ ID NO: 37 - protein, 12 aa, synthetic construct
  REGION 1..12: note = mAb-specific epitope
CQFDLSTRRLKC

SEQ ID NO: 38 - protein, 12 aa, synthetic construct
  REGION 1..12: note = mAb-specific epitope
CQYNLSSRALKC

SEQ ID NO: 39 - protein, 12 aa, synthetic construct
  REGION 1..12: note = mAb-specific epitope
CVWQRWQKSYVC

SEQ ID NO: 40 - protein, 12 aa, synthetic construct
  REGION 1..12: note = mAb-specific epitope
CMWDRFSRWYKC

SEQ ID NO: 41 - protein, 25 aa, synthetic construct
  REGION 1..25: note = mAb-specific epitope
SFVLNWYRMSPSNQTDKLAAFPEDR

SEQ ID NO: 42 - protein, 19 aa, synthetic construct
  REGION 1..19: note = mAb-specific epitope
SGTYLCGAISLAPKAQIKE

SEQ ID NO: 43 - protein, 557 aa, synthetic construct
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 44 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 45 - protein, 378 aa, synthetic construct
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 46 - protein, 335 aa, synthetic construct
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 47 - protein, 184 aa, synthetic construct
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 48 - protein, 993 aa, synthetic construct
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 49 - protein, 364 aa, synthetic construct; identical to SEQ ID NO: 22

SEQ ID NO: 50 - protein, 193 aa, synthetic construct
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 51 - protein, 943 aa, synthetic construct
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 52 - protein, 497 aa, synthetic construct
MQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 53 - protein, 118 aa, synthetic construct
  REGION 1..118: note = CD19-1 VH chain
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTV

SEQ ID NO: 54 - protein, 107 aa, synthetic construct
  REGION 1..107: note = CD19-1 VL chain
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 55 - protein, 119 aa, synthetic construct
  REGION 1..119: note = CD19-2 VH chain
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTV

SEQ ID NO: 56 - protein, 112 aa, synthetic construct
  REGION 1..112: note = CD19-2 VL chain
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELK

SEQ ID NO: 57 - protein, 115 aa, synthetic construct
QVQLQQPGAELVRPGASVKLSCKASGYTFTSYWMNWVKQRPDQGLEWIGRIDPYDSETHYNQKFKDKAILTVDKSSSTAYMQLSSLTSEDSAVYYCARGNWDDYWGQGTTLTVSS

SEQ ID NO: 58 - protein, 107 aa, synthetic construct
DVQITQSPSYLAASPGETITINCRASKSISKDLAWYQEKPGKTNKLLIYSGSTLQSGIPSRFSGSGSGTDFTLTISSLEPEDFAMYYCQQHNKYPYTFGGGTKLEIK

SEQ ID NO: 59 - protein, 118 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSS

SEQ ID NO: 60 - protein, 111 aa, synthetic construct
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELK

SEQ ID NO: 61 - protein, 113 aa, synthetic construct
MADYKDIVMTQSHKFMSTSVGDRVNITCKASQNVDSAVAWYQQKPGQSPKALIYSASYRYSGVPDRFTGRGSGTDFTLTISSVQAEDLAVYYCQQYYSTPWTFGGGTKLEIKR

SEQ ID NO: 62 - protein, 127 aa, synthetic construct
EVKLVESGGGLVQPGGSLSLSCAASGFTFTDYYMSWVRQPPGKALEWLALIRSKADGYTTEYSASVKGRFTLSRDDSQSILYLQMNALRPEDSATYYCARDAAYYSYYSPEGAMDYWGQGTSVTVSS

SEQ ID NO: 63 - protein, 119 aa, synthetic construct
VQLQESGPGLVKPSQSLSLTCSVTDYSITSGYYWNWIRQFPGNKLEWMGYISYDGSNNYNPSLKNRISITRDTSKNQFFLKLSSVTTEDTATYYCSRGEGFYFDSWGQGTTLTVSSARS

SEQ ID NO: 64 - protein, 113 aa, synthetic construct
DIMMSQSPSSLAVSVGEKFTMTCKSSQSLFFGSTQKNYLAWYQQKPGQSPKLLIYWASTRESGVPDRFTGSGSGTDFTLAISSVMPEDLAVYYCQQYYNYPWTFGGGTKLEIK

SEQ ID NO: 65 - protein, 115 aa, synthetic construct
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVS

SEQ ID NO: 66 - protein, 111 aa, synthetic construct
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 67 - protein, 117 aa, synthetic construct
QVQLQQSGPELVRPGTFVKISCKASGYTFTNYDINWVNQRPGQGLEWIGWIYPGDGSTKYNEKFKAKATLTADKSSSTAYLQLNNLTSENSAVYFCASGYEDAMDYWGQGTSVTVSS

SEQ ID NO: 68 - protein, 108 aa, synthetic construct
DIKMTQSPSSMYASLGERVIINCKASQDINSYLSWFQQKPGKSPKTLIYRANRLVDGVPSRFSGSGSGQDYSLTISSLEYEDMGIYYCLQYDEFPLTFGAGTKLELKR

SEQ ID NO: 69 - protein, 120 aa, synthetic construct
EVKLQESGPELVKPGASVKMSCKASGYKFTDYVVHWLKQKPGQGLEWIGYINPYNDGTKYNEKFKGKATLTSDKSSSTAYMEVSSLTSEDSAVYYCARDYRYEVYGMDYWGQGTSVTVSS

SEQ ID NO: 70 - protein, 115 aa, synthetic construct
DIVLTQSPTIMSASPGERVTMTCTASSSVNYIHWYQQKSGDSPLRWIFDTSKVASGVPARFSGSGSGTSYSLTISTMEAEDAATYYCQQWRSYPLTFGDGTRLELKRADAAPTVS

SEQ ID NO: 71 - protein, 118 aa, synthetic construct
QVQLQQPGAEVVKPGASVKMSCKASGYTFTSYYIHWIKQTPGQGLEWVGVIYPGNDDISYNQKFKGKATLTADKSSTTAYMQLSSLTSEDSAVYYCAREVRLRYFDVWGAGTTVTVSS

SEQ ID NO: 72 - protein, 113 aa, synthetic construct
NIMLTQSPSSLAVSAGEKVTMSCKSSQSVFFSSSQKNYLAWYQQIPGQSPKLLIYWASTRESGVPDRFTGSGSGTDFTLTISSVQSEDLAIYYCHQYLSSRTFGGGTKLEIKR

SEQ ID NO: 73 - protein, 115 aa, synthetic construct; identical to SEQ ID NO: 65

SEQ ID NO: 74 - protein, 111 aa, synthetic construct; identical to SEQ ID NO: 66

SEQ ID NO: 75 - protein, 117 aa, synthetic construct; identical to SEQ ID NO: 67

SEQ ID NO: 76 - protein, 108 aa, synthetic construct; identical to SEQ ID NO: 68

SEQ ID NO: 77 - protein, 120 aa, synthetic construct
EVQLQQSGPDLVKPGASVKISCKASGYSFTGYYMHWVKQSHGKSLEWIGRINPNNGVTLYNQKFKDKAILTVDKSSTTAYMELRSLTSEDSAVYYCARSTMITNYVMDYWGQVTSVTVSS

SEQ ID NO: 78 - protein, 108 aa, synthetic construct
SIVMTQTPTFLLVSAGDRVTITCKASQSVSNDVAWYQQKPGQSPTLLISYTSSRYAGVPDRFIGSGYGTDFTFTISTLQAEDLAVYFCQQDYNSPPTFGGGTKLEIKR

SEQ ID NO: 79 - protein, 119 aa, synthetic construct
QIQLVQSGPELKKPGETVKISCKASGYTFTNFGMNWVKQGPGEGLKWMGWINTNTGEPRYAEEFKGRFAFSLETTASTAYLQINNLKNEDTATYFCARDWDGAYFFDYWGQGTTLTVSS

SEQ ID NO: 80 - protein, 107 aa, synthetic construct
SIVMTQTPKFLLVSAGDRVTITCKASQSVSNDVAWYQQKPGQSPKLLINFATNRYTGVPNRFTGSGYGTDFTFTISTVQAEDLALYFCQQDYSSPWTFGGGTKLEIK

SEQ ID NO: 81 - protein, 117 aa, synthetic construct
QVQLQQSRPELVKPGASVKMSCKASGYTFTDYVISWVKQRTGQGLEWIGEIYPGSNSIYYNEKFKGRATLTADKSSSTAYMQLSSLTSEDSAVYFCAMGGNYGFDYWGQGTTLTVSS

SEQ ID NO: 82 - protein, 108 aa, synthetic construct
QIVLTQSPAIMSASLGERVTLTCTASSSVNSNYLHWYQQKPGSSPKLWIYSTSNLASGVPARFSGSGSGTSYSLTISSMEAEDAATYYCHQYHRSPLTFGAGTKLELK

SEQ ID NO: 83 - protein, 122 aa, synthetic construct
EVQLVESGGGLVQPKGSLKLSCAASGFTFNTYAMNWVRQAPGKGLEWVARIRSKSNNYATYYADSVKDRFTISRDDSQSMLYLQMNNLKTEDTAMYYCVRQWDYDVRAMNYWGQGTSVTVSS

SEQ ID NO: 84 - protein, 107 aa, synthetic construct
DIVMTQSHIFMSTSVGDRVSITCKASQDVDTAVAWYQQKPGQSPKLLIYWASTRLTGVPDRFTGSGSGTDFTLTISNVQSEDLADYFCQQYSSYPYTFGGGTKLEIK

SEQ ID NO: 85 - protein, 118 aa, synthetic construct
QVQLQQSGAELVRPGASVTLSCKASGYTFSDYEMHWVIQTPVHGLEWIGAIDPETGGTAYNQKFKGKAILTADKSSSTAYMELRSLTSEDSAVYYCTGYYDYDSFTYWGQGTLVTVSA

SEQ ID NO: 86 - protein, 107 aa, synthetic construct
DIVMTQSQKIMSTTVGDRVSITCKASQNVDAAVAWYQQKPGQSPKLLIYSASNRYTGVPDRFTGSGSGTDFTLTISNMQSEDLADYFCQQYDIYPYTFGGGTKLEIK

SEQ ID NO: 87 - protein, 119 aa, synthetic construct
EVKLVESGGGLVKPGGSLKLSCAASGFTFSSYAMSWVRQIPEKRLEWVASISRGGTTYYPDSVKGRFTISRDNVRNILYLQMSSLRSEDTAMYYCGRYDYDGYYAMDYWGQGTSVTVSS

SEQ ID NO: 88 - protein, 107 aa, synthetic construct
DIKMTQSPSSMYASLGERVTITCKASPDINSYLSWFQQKPGKSPKTLIYRANRLVDGVPSRFSGGGSGQDYSLTINSLEYEDMGIYYCLQYDEFPYTFGGGTKLEMK

SEQ ID NO: 89 - protein, 117 aa, synthetic construct
QVQLKESGPGLVAPSQTLSITCTVSGFSLTSYGVHWVRQPPGKGLEWLGVIWAGGFTNYNSALKSRLSISKDNSKSQVLLKMTSLQTDDTAMYYCARRGSSYSMDYWGQGTSVTVSS

SEQ ID NO: 90 - protein, 106 aa, synthetic construct
EIVLSQSPAITAASLGQKVTITCSASSNVSYIHWYQQRSGTSPRPWIYEISKLASGVPVRFSGSGSGTSYSLTISSMEAEDAAIYYCQQWNYPLITFGSGTKLEIQ

SEQ ID NO: 91 - protein, 116 aa, synthetic construct
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSS

SEQ ID NO: 92 - protein, 107 aa, synthetic construct
DIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIK

SEQ ID NO: 93 - protein, 118 aa, synthetic construct
QVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTV

SEQ ID NO: 94 - protein, 108 aa, synthetic construct
DIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKAL

SEQ ID NO: 95 - protein, 119 aa, synthetic construct
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 96 - protein, 112 aa, synthetic construct
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 97 - protein, 119 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 98 - protein, 112 aa, synthetic construct
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 99 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 100 - protein, 111 aa, synthetic construct
DIVLTGSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 101 - protein, 117 aa, synthetic construct
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGRGTTLTVSS

SEQ ID NO: 102 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 103 - protein, 119 aa, synthetic construct
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPDGNYWYFDVWGAGTTVTVSS

SEQ ID NO: 104 - protein, 107 aa, synthetic construct
DIVMTQSHKFMSTSVGDRVSITCKASQDVGIAVAWYQQKPGQSPKLLIYWASTRHTGVPDRFTGSGSGTDFTLTISNVQSEDLADYFCQQYSSYPYTFGGGTKLEIK

SEQ ID NO: 105 - protein, 120 aa, synthetic construct
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSS

SEQ ID NO: 106 - protein, 107 aa, synthetic construct
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 107 - protein, 121 aa, synthetic construct
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARGKVYYGSNPFAYWGQGTLVTVSA

SEQ ID NO: 108 - protein, 107 aa, synthetic construct
DIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIK

SEQ ID NO: 109 - protein, 120 aa, synthetic construct
QVQLQQSGPELVKPGASVKISCKASGYAFSSSWMNWVKQRPGQGLEWIGRIYPGDGDTKYNGKFKGKATLTADKSSSTAYMQLSSLTSVDSAVYFCARSTMIATGAMDYWGQGTSVTVSS

SEQ ID NO: 110 - protein, 107 aa, synthetic construct
ETTVTQSPASLSMAIGEKVTIRCITSTDIDDDMNWYQQKPGEPPKLLISEGNTLRPGVPSRFSSSGYGTDFVFTIENMLSEDVADYYCLQSDNLPLTFGGGTKLEIK

SEQ ID NO: 111 - protein, 120 aa, synthetic construct; identical to SEQ ID NO: 109

SEQ ID NO: 112 - protein, 108 aa, synthetic construct
DIVMTQSHKFMSTSVGDRVSITCKASQDVSTAVAWYQQKPGQSPKLLIYSASYRYTGVPDRFTGSGSGTDFTFTISSVQAEDLAVYYCQQHYSTPPYTFGGGTKLEIK

SEQ ID NO: 113 - protein, 141 aa, synthetic construct
MGWSWIFLFLVSGTGGVLSEVQLQQSGPELVKPGASVKMSCKASGYTFTDYYMKWVKQSHGKSLEWIGDIIPSNGATFYNQKFKGKATLTVDRSSSTAYMHLNSLTSEDSAVYYCTRSHLLRASWFAYWGQGTLVTVSAAS

SEQ ID NO: 114 - protein, 134 aa, synthetic construct
MESQTQVLMSLLFWVSGTCGDFVMTQSPSSLTVTAGEKVTMSCKSSQSLLNSGNQKNYLTWYLQKPGQPPKLLIYWASTRESGVPDRFTGSGSGTDFTLTISSVQAEDLAVYYCQNDYSYPYTFGGGTKLEIKR

SEQ ID NO: 115 - protein, 140 aa, synthetic construct
WTWRFLFVVAAATGVQSQVQLLQSGAEVKKPGSSVKVSCKASGGTFSTYAISWVRQAPGQGLEWMGGIIPIFGIVNYAQKFQGRVTITADESTSTAYMELSSLRSEDTAVYYCARGGGSGPDVLDIWGQGTMVTVSSAST

SEQ ID NO: 116 - protein, 132 aa, synthetic construct
MDMRVPAQLLGLLLLWLPGARCVIWMTQSPSLLSASTGDRVTISCRMSQGIRSYLAWYQQKPGKAPELLIYAASTLQSGVPSRFSGSGSGTDFTLTISSLQSEDFATYYCQQYYSFPYTFGQGTKLEIKRTV

SEQ ID NO: 117 - protein, 115 aa, synthetic construct; identical to SEQ ID NO: 57

SEQ ID NO: 118 - protein, 107 aa, synthetic construct; identical to SEQ ID NO: 58

SEQ ID NO: 119 - protein, 118 aa, synthetic construct; identical to SEQ ID NO: 59

SEQ ID NO: 120 - protein, 111 aa, synthetic construct; identical to SEQ ID NO: 60

SEQ ID NO: 121 - protein, 127 aa, synthetic construct; identical to SEQ ID NO: 62

SEQ ID NO: 122 - protein, 113 aa, synthetic construct; identical to SEQ ID NO: 61

SEQ ID NO: 123 - protein, 119 aa, synthetic construct; identical to SEQ ID NO: 63

SEQ ID NO: 124 - protein, 113 aa, synthetic construct; identical to SEQ ID NO: 64

SEQ ID NO: 125 - protein, 464 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 126 - protein, 498 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSCPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 127 - protein, 513 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSCPYSNPSLCSGGGGSCPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 128 - protein, 522 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 129 - protein, 500 aa, synthetic construct
GGGGSCPYSNPSLCSGGGGSCPYSNPSLCSGGGGSEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 130 - protein, 466 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGSCPYSNPSLCSGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 131 - protein, 472 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGGSCPYSNPSLCSGGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 132 - protein, 491 aa, synthetic construct
GGGGSCPYSNPSLCSGGGGSGGGGSEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGSCPYSNPSLCSGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 133 - protein, 497 aa, synthetic construct
GGGGSCPYSNPSLCSGGGGSGGGGSEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGGSCPYSNPSLCSGGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 134 - protein, 490 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGSCPYSNPSLCSGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 135 - protein, 496 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGGSCPYSNPSLCSGGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 136 - protein, 514 aa, synthetic construct
GGGGSCPYSNPSLCSGGGGSGGGGSEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 137 - protein, 515 aa, synthetic construct
GGGGSCPYSNPSLCSGGGGSGGGGSEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGSCPYSNPSLCSGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 138 - protein, 521 aa, synthetic construct
GGGGSCPYSNPSLCSGGGGSGGGGSEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGGSCPYSNPSLCSGGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 139 - protein, 504 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 140 - protein, 547 aa, synthetic construct
GGGGSCPYSNPSLCSGGGGSGGGGSEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 141 - protein, 523 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGSCPYSNPSLCSGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 142 - protein, 467 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 518 aa, synthetic construct
MTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 144 - protein, 16 aa, synthetic construct
  REGION 1..16: note = mAb-specific epitope
ELPTQGTFSNVSTNVS

SEQ ID NO: 145 - protein, 469 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 485 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGSGGGGSCPYSNPSLCTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 491 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 500 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGSGGGGSCPYSNPSLCSGGGGSCPYSNPSLCTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 506 aa, synthetic construct
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 136 aa, synthetic construct
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 151 - protein, 284 aa, synthetic construct
MLQMAGQCSQNEYFDSLLHACIPCQLRCSSNTPPLTCQRYCNASVTNSVKGTNAGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 152 - protein, 490 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 153 - protein, 496 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 154 - protein, 505 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSCPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 155 - protein, 511 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSCPYSNPSLCSGGGGSAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 156 - protein, 474 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGCPYSNPSLCGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 157 - protein, 480 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGGGCPYSNPSLCGGGGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 158 - protein, 487 aa, synthetic construct
GGGGSCPYSNPSLCGQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 159 - protein, 493 aa, synthetic construct
GGGGSCPYSNPSLCSGGGGSGQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 160 - protein, 502 aa, synthetic construct
GGGGSCPYSNPSLCSGGGGSCPYSNPSLCGQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 161 - protein, 508 aa, synthetic construct
GGGGSCPYSNPSLCSGGGGSCPYSNPSLCSGGGGSGQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 162 - protein, 558 aa, synthetic construct
GGGGSCPYSNPSLCSGGGGSGGGGSEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSGGGGSGGGGSGGGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRADPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 163 - protein, 536 aa, synthetic construct
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSSSGGSCPYSNPSLCSGGSDIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKRADPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 164 - protein, 554 aa, synthetic construct
GGGGSCPYSNPSLCSGGGGSGGGGSQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 165 - protein, 532 aa, synthetic construct
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSSSGGSCPYSNPSLCSGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKRSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 166 - protein, 553 aa, synthetic construct
GGGGSCPYSNPSLCSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 167 - protein, 531 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSSSGGSCPYSNPSLCSGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 168 - protein, 552 aa, synthetic construct
PGGGGSCPYSNPSLCSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 169 - protein, 529 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSSGGSCPYSNPSLCSGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSDPGSGGGGSCPYSNPSLCSGGGGSELPTQGTFSNVSTNVSPAKPTTTACPYSNPSLCAPTTTPAPRPPTPAPTIASQPLSLRPEACRPAAGGAVHTRGLDFACDIYIWAPLAGTCGVLLLSLVITLYCRRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRE

SEQ ID NO: 170 - protein, 124 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 171 - protein, 113 aa, synthetic construct
  REGION 1..113: note = Humanized F19 VL chain
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 172 - protein, 119 aa, synthetic construct
QVQLQQSGAELARPGASVNLSCKASGYTFTNNGINWLKQRTGQGLEWIGEIYPRSTNTLYNEKFKGKATLTADRSSNTAYMETELRSLTSEDSAVYFCARTLTAPFAFWGQGTLVTVSA

SEQ ID NO: 173 - protein, 107 aa, synthetic construct
QIVLTQSPAIMSASPGEKVTMTCSASSGVNFMHWYQQKSGTSPKRWIFDTSKLASGVPARFSGSGSGTSYSLTISSMEAEDAATYYCQQWSFNPPTFGGGTKLEIKR

SEQ ID NO: 174 - protein, 12 aa, synthetic construct
  REGION 1..12: note = mAb-specific epitope
GQNDTSQTSSPS

SEQ ID NO: 175 - protein, 112 aa, synthetic construct
  REGION 1..112: note = CD3zeta intracellular signaling domain (ISD)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 176 - protein, 42 aa, synthetic construct
  REGION 1..42: note = 41BB intracellular signaling domain (ISD)
KRGRKKLLYIFKQPFMRPVQTTQEEDGCSCRFPEEEEGGCEL

SEQ ID NO: 177 - protein, 42 aa, synthetic construct; identical to SEQ ID NO: 176

SEQ ID NO: 178 - protein, 21 aa, synthetic construct
MALPVTALLLPLALLLHAARP

SEQ ID NO: 179 - protein, 16 aa, synthetic construct
GLAVSTISSFFPPGYQ

SEQ ID NO: 180 - protein, 45 aa, synthetic construct
TTTPAPRPPTPAPTIASQPLSLRPEACRPAAGGAVHTRGLDFACD

SEQ ID NO: 181 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 24 aa, synthetic construct
IYIWAPLAGTCGVLLLSLVITLYC

SEQ ID NO: 183 - protein, 52 aa, synthetic construct
FFIPLLVVILFAVDTGLFISTQQQVTFLLKIKRTRKGFRLLNPHPKPNPKNN

SEQ ID NO: 184 - protein, 215 aa, synthetic construct
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPE

SEQ ID NO: 185 - protein, 41 aa, synthetic construct
RSKRSRGGHSDYMNMTPRRPGPTRKHYQPYAPPRDFAAYRS

SEQ ID NO: 186 - protein, 113 aa, synthetic construct
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTV

SEQ ID NO: 187 - protein, 108 aa, synthetic construct
EIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIK

(19) United States
(12) Patent Application Publication
(10) Pub. No.: US 2024/0228636 A1
(43) Pub. Date: Jul. 11, 2024

(54) mAb-DRIVEN CHIMERIC ANTIGEN RECEPTOR SYSTEMS FOR SORTING/DEPLETING ENGINEERED IMMUNE CELLS

(71) Applicants: Cellectis, Paris (FR); Allogene Therapeutics, Inc., South San Francisco, CA (US)

(72) Inventors: Barbara Johnson SASU, San Francisco, CA (US); Arvind RAJPAL, San Francisco, CA (US); Philippe DUCHATEAU, Draveil (FR); Alexandre JUILLERAT, New York, NY (US); Julien VALTON, New York, NY (US)

(21) Appl. No.: 18/481,797

(22) Filed: Oct. 5, 2023

Related U.S. Application Data
(63) Continuation of application No. 15/546,630, filed on Jul. 26, 2017, now abandoned.

(30) Foreign Application Priority Data
Jan. 26, 2015 (DK) PA20157004

(51) Int. Cl. C07K 16/28; A61K 35/17; A61K 39/00; A61K 39/395; C07K 14/705; C07K 14/725; C07K 14/735; C07K 16/30; C12N 5/00; C12N 5/0783; G01N 15/01; G01N 15/10; G01N 15/1031; G01N 15/14; G01N 15/149

(52) U.S. Cl. CPC C07K 16/2866; A61K 35/17; A61K 39/0011; A61K 39/001176; A61K 39/39558; C07K 14/7051; C07K 14/70517; C07K 14/70535; C07K 14/70578; C07K 16/2803; C07K 16/2887; C07K 16/3061; C12N 5/0093; C12N 5/0636; C12N 5/0638; G01N 15/1031; G01N 15/14; A61K 2039/505; A61K 2039/5156; A61K 2039/5158; A61K 2039/6056; A61K 2039/64; C07K 2317/24; C07K 2317/53; C07K 2317/56; C07K 2317/622; C07K 2319/00; C07K 2319/02; C07K 2319/03; C12N 2510/00; G01N 2015/016; G01N 2015/1006; G01N 2015/1028; G01N 15/149

(57) ABSTRACT

A polypeptide encoding a chimeric antigen receptor (CAR) comprising at least one extracellular binding domain that comprises a scFv formed by at least a VH chain and a VL chain specific to an antigen, wherein said extracellular binding domain comprises at least one mAb-specific epitope.

### SEQUENCE LISTING

The text of the computer readable sequence listing filed herewith, titled “CELLT-37390.302”, created Mar. 15, 2024, having a file size of 222,952 bytes, is hereby incorporated by reference in its entirety.

### FIELD OF THE INVENTION

The present invention relates to improved chimeric antigen receptors (CAR) to be used in immunotherapy, the extracellular binding domains (scFv) of which have been modified by insertion of a mAb-specific epitope to allow both sorting and/or depletion of the immune cells endowed with said CARs. The present invention relates also to the immune cells expressing said CARs, to the methods of in vivo depleting and/or in vitro sorting said CAR-expressing immune cells, and is drawn to the their therapeutic use.

### BACKGROUND

Adoptive immunotherapy, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. 2011). Transfer of viral antigen specific T cells is a well-established procedure used for the treatment of transplant associated viral infections and rare viral-related malignancies. Similarly, isolation and transfer of tumor specific T cells has been shown to be successful in treating melanoma.

Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and heavy variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), ICOS and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010).

However, despite their unprecedent efficacy for tumor eradication in vivo, CAR T cells can promote acute adverse events after being transferred into patients. Among the well documented adverse events is Graft versus host disease (GvHD), on-target off-tumor activity or aberrant lymphoproliferative capacity due to vector derived insertional mutagenesis. Therefore, there is a need to develop cell specific depletion systems to prevent such deleterious events to occur in vivo.

There are many on-going researches to develop a safer CAR-based immunotherapy, such as on inhibitory signals referred to as immune checkpoints (such as CTLA-4- or PD-1) which are crucial for the maintenance of self-tolerance and also to limit immune-mediated collateral tissue damage (Dolan et al, 2014). Recently, inhibitory chimeric antigen receptors (iCARs) were designed having as objective to put the brakes on T cell function upon encountering off-target cells (Federov et al. 2013). Another system is described in Budde et al. (2013) in which a CD20 Chimeric Antigen Receptor is combined with an inducible caspase 9 (iC9) suicide switch. In the application US 2014/0286987, the latter gene is made functional in the presence of the prodrug AP1903 (tacrolimus) by binding to the mutated FK506-binding protein (FKBP1). A clinical trial is ongoing sponsored by the company Bellicum in which the above capsase technology (CaspaCIDe™) is engineered into GD2 targeted third generation CAR T cells. A similar apoptosis-inducing system based on a multimerizing agent is described in the application WO 2014/152177.

Philip et al (2014) describes the RQR8 system which is being used as compact marker/suicide gene allowing selection of transduced cells. RQR8 derives from the combination of target epitopes from both CD34 and CD20 antigens. This construct allows selection with the clinically approved CliniMACS CD34 system (Miltenyi). Moreover, this RQR8 construct binds the widely used pharmaceutical antibody rituximab, resulting in selective deletion of transgene-expressing cells. Within this system, RQR8 is co-expressed with a CAR in a retroviral vector using the foot-and-mouth disease 2A peptide, resulting thereby into the expression of 2 independent transgenes (RQR8 and CAR) on the surface of the T-cells. This system presents some limitations from the industrial perspective, as first, it requires the cloning large retroviral inserts, and second, to ensure that the transformed cells express both RQR8 and CAR polypeptides, to eliminate possible “false-positive” i.e. T-cells that would not express both polypeptides, in particular the RQR8 suicide gene allowing the depletion of the engineered immune cells in the event of undesirable effects.

The concept of depleting T cells in the context of auto-immune disease and transplantation has been successfully practiced in the clinic for decades. To deplete cell-mediated immunity, including T-cells, immunosuppressive drugs such as glucocorticoids or cytostatics such as alkylating agents (cyclophosphamide, nitrosoureas, platinum compounds . . . ) or antimetabolites (methotrexate, azathioprine, fluorouracil . . . ) are widely used. However, despite their immunosuppressive efficacy, these drugs are not discriminative as they affect the proliferation of all T and B cells. Antibodies are sometimes used as a quick and potent immunosuppressive therapy to prevent the acute rejection reactions as well as a targeted treatment of lymphoproliferative or autoimmune disorders, in particular anti-CD20 monoclonals. In vivo elimination of T cell subsets was performed by Benjamin and Waldmann (1986) to determine the role of CD4+ T cells in generating antibody responses to soluble proteins, and by Cobbold et al. (1986) to determine the role of CD4+ and CD8+ T cells in rejecting bone marrow and tissue allografts. In vivo depletion has been performed extensively to study varied topics including control of antiviral cytotoxic T lymphocyte (CTL) responses (Buller et al., 1987). However, the antibodies which have been used so far on T cells direct antigens (CD3, CD4, CD52) that are all broadly present on resting or activated T cells as well as on other cell types. As such, the use of such antibodies would not allow the selective elimination of the engineered immune cells endowed with CARs.

As presented thereafter, the inventors have sought for an “all-in-one” system which allows an optimized in vitro sorting of CAR-expressing immune cells by reducing “false-positive”, meanwhile allowing the in vivo depletion of the immune cells expressing said CARs in case of adverse clinical event.

### SUMMARY OF THE INVENTION

The present invention is drawn to chimeric antigen receptors (CAR), which extracellular binding domain (scFv) is modified in such a way to allow both cell sorting and cell depletion (see FIG. 2 for illustrative embodiment). This structure named mAb-driven sorting/depletion system consists in inserting a selected epitope within the scFv; this epitope having a specificity to be recognized by a specific antibody (preferably mAb). Given the fact that mainly the external ligand binding domain of the CAR is modified to include the epitope, different CAR architectures can be envisioned: single-chain or multi-chain. The chimeric scFv of the invention, which is formed of the VH and VL polypeptides and the specific epitope(s) may itself have different structures depending on the position of insertion of the epitope and the use of linkers. The present invention also relates to the resulting method for sorting and/or depleting the engineered immune cells endowed with the modified CARs.

Several epitope-mAb couples can be used to generate such system; in particular those already approved for medical use, such as CD20/rituximab as a non-limiting example.

To further enhance the cytotoxicity of the engineered immune cells, the epitope-specific antibody may be conjugated with a cytotoxic drug. It is also possible to promote CDC cytotoxicity by using engineered antibodies on which are grafted component(s) of the complement system.

Finally, the invention encompasses therapeutic methods where the activation of the engineered immune cells endowed with CARs is modulated by depleting the cells by using an antibody that directs the external ligand binding domain of said CARs.

The invention can be summarized by the following items:
1. A polypeptide encoding a chimeric antigen receptor (CAR) comprising at least one extracellular binding domain that comprises a scFv formed by at least a VH chain and a VL chain specific to an antigen, wherein said extracellular binding domain comprises at least one mAb-specific epitope.
2. The polypeptide according to item 1, wherein said mAb-specific epitope is located between the VH and VL chains.
3. The polypeptide according to item 1 or 2, wherein said VH and VL chains, and mAb specific-epitope are bound together by at least one linker and to the transmembrane domain of said CAR by a hinge.
4. The polypeptide according to item 3, wherein the mAb-epitope is joined to the VH and VL chains by two linkers.
5. The polypeptide according to any one of items 1 to 3 wherein the mAb-specific epitope is an epitope to be bound by an epitope-specific mAb for in vitro cell sorting and/or in vivo cell depletion of T cells expressing a CAR comprising such epitope.
6. The polypeptide according to any one of items 1 to 5, wherein the polypeptide comprises one extracellular binding domain, wherein said extracellular binding domain further comprises a hinge, and said polypeptide further comprises
   a transmembrane domain, and,
   an intracellular domain.
7. The polypeptide according to any one of items 1 to 6, wherein the extracellular binding domain comprises 1, 2, 3, 4, 5, 6, 7, 8, 9 or 10 mAb-specific epitopes.
8. The polypeptide according to any one of items 1 to 7, wherein the extracellular binding domain comprises 1, 2, 3 or, 4 mAb-specific epitopes.
9. The polypeptide according to any one of items 1 to 8, wherein the extracellular binding domain comprises 2, 3 or, 4 mAb-specific epitopes.
10. The polypeptide according to any one of items 1 to 9, wherein the extracellular binding domain comprises the following sequence
V₁-L₁-V₂-(L)ₓ-Epitope1-(L)ₓ-;
V₁-L₁-V₂-(L)ₓ-Epitope1-(L)ₓ-Epitope2-(L)ₓ-;
V₁-L₁-V₂-(L)ₓ-Epitope1-(L)ₓ-Epitope2-(L)ₓ-Epitope3-(L)ₓ-;
(L)ₓ-Epitope1-(L)ₓ-V₁-L₁-V₂;
(L)ₓ-Epitope1-(L)ₓ-Epitope2-(L)ₓ-V₁-L₁-V₂;
Epitope1-(L)ₓ-Epitope2-(L)ₓ-Epitope3-(L)ₓ-V₁-L₁-V₂;
(L)ₓ-Epitope1-(L)ₓ-V₁-L₁-V₂-(L)ₓ-Epitope2-(L)ₓ;
(L)ₓ-Epitope1-(L)ₓ-V₁-L₁-V₂-(L)ₓ-Epitope2-(L)ₓ-Epitope3-(L)ₓ-;
(L)ₓ-Epitope1-(L)ₓ-V₁-L₁-V₂-(L)ₓ-Epitope2-(L)ₓ-Epitope3-(L)ₓ-Epitope4-(L)ₓ-;
(L)ₓ-Epitope1-(L)ₓ-Epitope2-(L)ₓ-V₁-L₁-V₂-(L)ₓ-Epitope3-(L)ₓ-;
(L)ₓ-Epitope1-(L)ₓ-Epitope2-(L)ₓ-V₁-L₁-V₂-(L)ₓ-Epitope3-(L)ₓ-Epitope4-(L)ₓ-;
V₁-(L)ₓ-Epitope1-(L)ₓ-V₂;
V₁-(L)ₓ-Epitope1-(L)ₓ-V₂-(L)ₓ-Epitope2-(L)ₓ;
V₁-(L)ₓ-Epitope1-(L)ₓ-V₂-(L)ₓ-Epitope2-(L)ₓ-Epitope3-(L)ₓ;
V₁-(L)ₓ-Epitope1-(L)ₓ-V₂-(L)ₓ-Epitope2-(L)ₓ-Epitope3-(L)ₓ-Epitope4-(L)ₓ;
(L)ₓ-Epitope1-(L)ₓ-V₁-(L)ₓ-Epitope2-(L)ₓ-V₂; or,
(L)ₓ-Epitope1-(L)ₓ-V₁-(L)ₓ-Epitope2-(L)ₓ-V₂-(L)ₓ-Epitope3-(L)ₓ;
wherein,
V₁ is V₁ and V₂ is V_{H} or V₁ is V_{H} and V₂ is V_{L};
L₁ is a linker suitable to link the V_{H} chain to the V₁ chain;
L is a linker comprising glycine and serine residues, and each occurrence of L in the extracellular binding domain can be identical or different to other occurrence of L in the same extracellular binding domain, and,
x is 0 or 1 and each occurrence of x is selected independently from the others; and, Epitope 1, Epitope 2 and Epitope 3 are mAb-specific epitopes and can be identical or different.
11. The polypeptide according to item 10, wherein the extracellular binding domain comprises the following sequence
V₁-L₁-V₂-L-Epitope1; V₁-L₁-V₂-L-Epitope1-L; V₁-L₁-V₂-L-Epitope1-L-Epitope2; V₁-L₁-V₂-L-Epitope1-L-Epitope2-L; V₁-L₁-V₂-L-Epitope1-L-Epitope2-L-Epitope3; V₁-L₁-V₂-L-Epitope1-L-Epitope2-L-Epitope3-L; V₁-L₁-V₂-Epitope1; V₁-L₁-V₂-Epitope1-L; V₁-L₁-V₂-Epitope1-L-Epitope2; V₁-L₁-V₂-Epitope1-L-Epitope2-L; V₁-L₁-V₂-Epitope 1-L-Epitope2-L-Epitope3; V₁-L₁-V₂-Epitope1-L-Epitope2-L-Epitope3-L; Epitope1-V₁-L₁-V₂; Epitope1-L-V₁-L₁-V₂; L-Epitope1-V₁-L₁-V₂; L-Epitope1-L-V₁-L₁-V₂; Epitope1-L-Epitope2-V₁-L₁-V₂; Epitope1-L-Epitope2-L-V₁-L₁-V₂; L-Epitope1-L-Epitope2-V₁-L₁-V₂; L-Epitope1-L-Epitope2-L-V₁-L₁-V₂; Epitope1-L-Epitope2-L-Epitope3-V₁-L₁-V₂; Epitope1-L-Epitope2-L-Epitope3-L-V₁-L₁-V₂; L-Epitope1-L-Epitope2-L-Epitope3-V₁-L₁-V₂; L-Epitope1-L-Epitope2-L-Epitope3-L-V₁-L₁-V₂; V₁-L-Epitope1-L-V₂; L-Epitope1-L-V₁-L-Epitope2-L-V₂; V₁-L-Epitope1-L-V₂-L-Epitope2-L; V₁-L-Epitope1-L-V₂-L-Epitope2-L-Epitope3; V₁-L-Epitope1-L-V₂-L-Epitope2-Epitope3; V₁-L-Epitope1-L-V₂-L-Epitope2-L-Epitope3-Epitope4; L-Epitope1-L-V₁-L-Epitope2-L-V₂-L-Epitope3-L; Epitope1-L-V₁-L-Epitope2-L-V₂-L-Epitope3-L; L-Epitope1-L-V₁-L-Epitope2-L-V₂-L-Epitope3; L-Epitope1-L-V₁-L₁-V₂-L-Epitope2-L; L-Epitope 1-L-V₁-L₁-V₂-L-Epitope2-L-Epitope3; L-Epitope1-L-V₁-L₁-V₂-L-Epitope2-Epitope3, or Epitope1-L-V₁-L₁-V₂-L-Epitope2-L-Epitope3-Epitope4 wherein
V₁ is V₁ and V₂ is V_{H} or V₁ is V_{H} and V₂ is VL;
L₁ is any linker suitable to link the V_{H} chain to the V₁ chain;
L is a linker comprising glycine and serine residues, and each occurrence of L in the extracellular binding domain can be identical or different to other occurrences of L in the same extracellular binding domain, and,

Epitope 1, Epitope 2 and Epitope 3 are mAb-specific epitopes and can be identical or different.
12. The polypeptide according to item 10, wherein L₁ is a linker comprising Glycine and/or Serine.
13. The polypeptide according to item 12, wherein L₁ is a linker comprising the amino acid sequence (Gly-Gly-Gly-Ser)ₙ or (Gly-Gly-Gly-Gly-Ser)ₙ, where n is 1, 2, 3, 4 or 5 or a linker comprising the amino acid sequence (Gly₄Ser)₄ or (Gly₄Ser)₃.
14. The polypeptide according to any one of items 10 to 13 wherein L is a linker comprising Glycine and/or Serine.
15. The polypeptide according to item 14 wherein L is a linker having an amino acid sequence selected from SGG, GGS, SGGS, SSGGS, GGGG, SGGGG, GGGGS, SGGGGS, GGGGGS, SGGGGGS, SGGGGG, GSGGGGS, GGGGGGGS, SGGGGGGG, SGGGGGGGS, or SGGGGSGGGGS.
16. The polypeptide according to item 14 wherein L is a SGGGG, GGGGS or SGGGGS.
17. The polypeptide according to any one of items 10 to 16 wherein Epitope 1, Epitope 2, Epitope 3 and Epitope 4 are independently selected from mAb-specific epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.
18. The polypeptide according to any one of items 10 to 16 wherein Epitope 1, Epitope 2, Epitope 3 and Epitope 4 are independently selected from mAb-specific epitopes having an amino acid sequence of SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 144 or SEQ ID NO 174.
19. The polypeptide according to any one of items 10 to 18 wherein Epitope 1 is a mAb-specific epitope having an amino acid sequence of SEQ ID NO 35.
20. The polypeptide according to any one of items 10 to 19 wherein Epitope 2 is a mAb-specific epitope having an amino acid sequence of SEQ ID NO 35.
21. The polypeptide according to any one of items 10 to 20 wherein Epitope 3 is a mAb-specific epitope having an amino acid sequence of SEQ ID NO 35 or SEQ ID NO 144.
22. The polypeptide according to any one of items 10 to 21 wherein Epitope 4 is a mAb-specific epitope having an amino acid sequence of SEQ ID NO 35.
23. The polypeptide according to item 22 wherein Epitope 1, Epitope 2 and Epitope 4 are a mAb-specific epitope having an amino acid sequence of SEQ ID NO 35 and Epitope 3 is a mAb-specific epitope having an amino acid sequence of SEQ ID NO 144.
24. The polypeptide according to any one of items 1 to 9, wherein the mAb-specific epitope is from one polypeptide selected from those listed in Table 1.
25. The polypeptide according to any one of items 1 to 9 wherein the mAb-specific epitope is selected from mAb-specific epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.
26. The polypeptide according to any one of items 1 to 9 wherein the mAb-specific epitope is selected from mAb-specific epitope having an amino acid sequence of SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 144 or SEQ ID NO 174.
27. The polypeptide according to any one of items 1 to 9 wherein the mAb-specific epitope is has an amino acid sequence of SEQ ID NO 35.
28. The polypeptide according to anyone of items 1 to 27, wherein said VH and VL chains have an antigenic target sequence of over 80% identity, preferably over 90%, and more preferably over 95% with SEQ ID NO 43 (CD19 antigen), SEQ ID NO 44 (CD38 antigen), SEQ ID NO 45 (CD123 antigen), SEQ ID NO 46 (CS1 antigen), SEQ ID NO 47 (BCMA antigen), SEQ ID NO 48 (FLT-3 antigen), SEQ ID NO 49 (CD33 antigen), SEQ ID NO 50 (CD70 antigen), SEQ ID NO 51 (EGFR-3v antigen) and SEQ ID NO 52 (WT1 antigen).
29. The polypeptide according to any one of items 1 to 27 wherein said antigen is a cell surface marker antigen.
30. The polypeptide according to any one of items 1 to 27 wherein said antigen is a tumor-associated surface antigen.
31. The polypeptide according to any one of items 1 to 27 wherein said antigen is selected from ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, GD3, C-type lectin-like molecule-1 (CLL-1), ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, survivin and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap), LRP6, melamona-associated Chondroitin Sulfate Proteoglycan (MCSP), CD38/CS1, MART1, WT1, MUC1, LMP2, Idiotype, NY-ESO-1, Ras mutant, gp100, proteinase 3, bcr-abl, tyrosinase, hTERT, EphA2, ML-TAP, ERG, NA17, PAX3, ALK, Androgen receptor; a lineage-specific or tissue specific antigen such as CD3, CD4, CD8, CD24, CD25, CD33, CD34, CD70, CD79, CD116, CD117, CD135, CD123, CD133, CD138, CTLA-4, B7-1 (CD80), B7-2 (CD86), endoglin, a major histocompatibility complex (MHC) molecule, BCMA (CD269, TNFRSF 17) or FLT-3.
32. The polypeptide according to any one of items 1 to 27 wherein VH and VL are selected from a VH of SEQ ID NO 65 and a VL of SEQ ID NO 66; a VH of SEQ ID NO 67 and a VL of SEQ ID NO 68; a VH of SEQ ID NO 69 and a VL of SEQ ID NO 70; a VH of SEQ ID NO 71 and a VL of SEQ ID NO 72; a VH of SEQ ID NO 77 and a VL of SEQ ID NO 78; a VH of SEQ ID NO 79 and a VL of SEQ ID NO 80; a VH of SEQ ID NO 81 and a VL of SEQ ID NO 82; a VH of SEQ ID NO 83 and a VL of SEQ ID NO 84; a VH of SEQ ID NO 85 and a VL of SEQ ID NO 86; a VH of SEQ ID NO 87 and a VL of SEQ ID NO 88; a VH of SEQ ID NO 89 and a VL of SEQ ID NO 90; a VH of SEQ ID NO 91 and a VL of SEQ ID NO 92; a VH of SEQ ID NO 93 and a VL of SEQ ID NO 94; a VH of SEQ ID NO 95 and a VL of SEQ ID NO 96; a VH of SEQ ID NO 97 and a VL of SEQ ID NO 98; a VH of SEQ ID NO 99 and a VL of SEQ ID NO 100; a VH of SEQ ID NO 101 and a VL of SEQ ID NO 102; a VH of SEQ ID NO 103 and a VL of SEQ ID NO 104; a VH of SEQ ID NO 105 and a VL of SEQ ID NO 106; a VH of SEQ ID NO 107 and a VL of SEQ ID NO 108; a VH of SEQ ID NO 109 and a VL of SEQ ID NO 110; a VH of SEQ ID NO 111 and a VL of SEQ ID NO 112; a VH of SEQ ID NO 113 and a VL of SEQ ID NO 114; a VH of SEQ ID NO 115 and a VL of SEQ ID NO 116; a VH of SEQ ID NO 117 and a VL of SEQ ID NO 118; a VH of SEQ ID NO 119 and a VL of SEQ ID NO 120; a VH of SEQ ID NO 121 and a VL of SEQ ID NO 122; or, a VH of SEQ ID NO 123 and a VL of SEQ ID NO 124, a VH of SEQ ID NO 170 and a VL of SEQ ID NO 171; a VH of SEQ ID NO 172 and a VL of SEQ ID NO 173; or, a VH of SEQ ID NO 174 and a VL of SEQ ID NO 175.
33. The polypeptide according to any one of items 2 to 32 wherein the hinge comprises a PD-1 hinge, an IgG4 hinge, a CD8alpha hinge or a FcγRIII alpha hinge.
34. The polypeptide according to any one of items 2 to 33 wherein the transmembrane domain comprises the transmembrane region(s) of the alpha, beta or zeta chain of the T-cell receptor, PD-1, 4-1BB, OX40, ICOS, CTLA-4, LAG3, 2B4, BTLA4, TIM-3, TIGIT, SIRPA, CD28, CD3 epsilon, CD45, CD4, CD5, CD8, CD9, CD16, CD22, CD33, CD37, CD64, CD80, CD86, CD134, CD137 or CD154.
35. The polypeptide according to any one of items 2 to 33 wherein the transmembrane domain comprises the transmembrane region(s) of PD-1 or CD8 alpha.
36. The polypeptide according to any one of items 2 to 33 wherein the transmembrane domain comprises the transmembrane region(s) of CD8 alpha.
37. The polypeptide according to any one of items 2 to 37 wherein the intracellular domain comprises a CD3zeta signalling domain.
38. The polypeptide according to any one of items 2 to 37 wherein the intracellular domain comprises a 4-1BB domain.
39. A polypeptide according to any one of items 1-38, wherein the CAR is a single-chain CAR.
40. A polypeptide according to item 1, wherein the said polypeptide shares over 80% identity, over 90%, over 95% with or is identical to SEQ ID NO 1 to 10, SEQ ID NO 125 to 141 or SEQ ID no 145 to 150 or SEQ ID NO 152 to 169.
41. A polypeptide according to any one of items 1-38 wherein the CAR is a multi-chain CAR.
42. A polynucleotide encoding a polypeptide according to any one of items 1 to 41.
43. A polynucleotide encoding a chimeric antigen receptor according to any one of items 1 to 41, wherein said CAR comprises a CD3 zeta signaling domain and co-stimulatory domain from 4-1BB.
44. An expression vector comprising a nucleic acid of item 42 or 43.
45. An engineered immune cell expressing at its cell surface a polypeptide according to any one of items 1 to 41.
46. The engineered immune cell according to item 45, wherein said cell is derived from inflammatory T-lymphocytes, cytotoxic T-lymphocytes, regulatory T-lymphocytes or helper T-lymphocytes.
47. The engineered immune cell according to item 45 or 46 for use as a medicament.
48. A method for engineering an immune cell of any one of items 45-47, comprising:
(a) Providing an immune cell;
(b) Introducing into said cell at least one polynucleotide encoding the chimeric antigen receptor according to any one of items 1-41.
(c) Expressing said polynucleotide into said cell.
49. The method for engineering an immune cell of item 48, wherein immune cell is a T-cell.
50. A method for in vitro sorting engineered immune cell expressing at its cell surface a polypeptide comprising at least one mAb-specific epitope according to any one of items 1 to 41 comprising
   contacting a population of immune cells comprising said engineered immune cells with a monoclonal antibody specific for the mAb-specific epitope;
   selecting the cells that bind to the monoclonal antibody to obtain a population of cells enriched in engineered immune cell.
51. The method according to item 50 wherein the monoclonal antibody specific for the mAb-specific epitope is conjugated to a fluorophore and the step of selecting the cells that bind to the monoclonal antibody is done by Fluorescence Activated Cell Sorting (FACS).
52. The method according to item 50 wherein the monoclonal antibody specific for the mAb-specific epitope is conjugated to a magnetic particle and the step of selecting the cells that bind to the monoclonal antibody is done by Magnetic Activated Cell Sorting (MACS).
53. The method according to any one of items 50 to 52 wherein the polypeptide comprises an mAb-specific epitope having an amino acid sequence of SEQ ID NO 35 and the monoclonal antibody is rituximab.
54. The method according to any one of items 50 to 52 wherein the polypeptide comprises an mAb-specific epitope having an amino acid sequence of SEQ ID NO 144 and the antibody used to contact the population of immune cells is QBEND-10.
55. The method according to any one of items 50 to 54 wherein the population of cells enriched in engineered immune cell comprises at least 70%, 75%, 80%, 85%, 90%, 95% of CAR-expressing immune cells.
56. A method for in vivo depleting an engineered immune cell expressing at its cell surface a polypeptide comprising at least one mAb-specific epitope according to any one of items 1 to 41 in a patient, comprising contacting said engineered immune cell with at least one epitope-specific mAb.
57. The method according to item 56 wherein the mAb-specific epitope is a CD20 epitope or mimotope and the epitope-specific mAb is rituximab.
58. The method according to item 57 wherein the mAb-specific epitope has an amino acid sequence of SEQ ID NO 35.
59. The method according to any one of items 56 to 58 wherein the epitope-specific mAb is conjugated by a molecule able to activate the complement system.
60. The method according to any one of items 56 to 59 wherein, wherein a cytotoxic drug is coupled to the epitope-specific mAb.
61. A method for in vivo depleting an engineered immune cell expressing at its cell surface a polypeptide comprising at least one mAb-specific epitope according to any one of items 1 to 41 in a patient, comprising contacting said engineered immune cell with bi-specific mAb (BsAb) able to bind both the mAb-specific epitope borne on said cells and to an surface antigen borne on an effector (and cytotoxic) cell.
62. A method according to any one of 48 to 61, wherein said immune cell is a T-cell.

### BRIEF DESCRIPTION OF THE FIGURES AND TABLE

FIG. 1: Schematic structure of the mAb-driven sorting/depletion system of the invention using here a single-chain CAR scaffold; several configurations are presented for the chimeric scFv with different positions of the VH, VL chains and the mAb-specific epitope.

FIG. 2: Schematic representation of cell sorting and cell depletion functioning by using the mAb-driven system of the invention. The addition of specific mAb (+/−complement) allows purification of CAR+ T cells by recognizing its epitope within the chimeric scFv. During the cell depletion step, the same specific mAb (+/−complement) by binding to its specific epitope within the chimeric scFv provokes a specific lysis of CAR+ T cells.

FIG. 3: Schematic representation of cell depletion using bi-specific antibody. By binding to both CAR-expressing immune cell and to an effector cell, this system allows recruitment of effector cells at the surface of CAR-expressing immune cell and triggers their specific depletion in vivo.

FIG. 4: CAR architecture for 10 CARs expressing anti-CD123 scFv with CD20 mimotope(s) used in Examples 1-2. A series of 10 chimeric scFv are designed in which one or two copies of the CD20 mimotopes (black box named “mimotope”) are inserted between the anti-CD123 scFv and the hinge. As depicted in the FIG. 4, all the 10 CARs have the same hinge (CD8 hinge), transmembrane domain (CD8 TM), co-stimulatory domain (4-1BB) and stimulatory domain (ITAM CD3 zeta). SEQ ID NO 1-10 comprise the leader sequence MALPVTALLLPLALLLHAARP, which is present when the CAR is initially expressed but which is not part of the CAR expressed at the surface of the cell. Depending on the position of the mimotope(s) in view of the scFv, 3 series of CARs are designed (Anti-CD123 No 1, 2, 3, 4, 5, 6, 7, 8, 9 and 10 corresponding to SEQ ID NO: 1, 2, 3, 4, 5, 6, 7, 8, 9 and 10, respectively):
first series: SEQ ID NO 1-2 and SEQ ID NO 3-4 correspond to the conformations wherein respectively one and two CD20 mimotope(s) is(are) inserted between the anti-CD123 scFv and the hinge; in SEQ ID NO 2 a GS linker joins the CD20 mimotope to the hinge. In SEQ ID NO 3-4, a GS linker interspaces the two mimotopes and in SEQ ID NO 4 has an extra GS linker between the mimotopes and the hinge.
second series: SEQ ID NO 5-6 correspond to the conformations wherein one copy of CD20 mimotope is inserted within the anti-CD123 scFv; the differences of sequence coming from the presence of respectively a short GS linker (SEQ ID NO 5) and a long GS linker (SEQ ID NO 6) lying on both sides.
third series: SEQ ID NO 7 to 10 correspond to the conformations wherein the anti-CD123 scFv is located between the CD mimotope(s) and the hinge. In SEQ ID NO. 7-8 and SEQ ID NO. 9-10 respectively, one copy and two copies of CD20 mimotope is (are) inserted. A GS linker is inserted between the 2 CD20 mimotopes, and for SEQ ID NO.10, a supplementary GS linker joins the mimotopes to the anti-CD123 scFv.

FIG. 5 shows the cytolytic activity of T cells expressing anti-CD123 CAR of SEQ ID NO 1-4 or 142 or control T Cell not expressing any anti CD123 CAR (mock T-cell-transfection step without any mRNA). The cytolytic activity is expressed as the frequency of specific cell lysis as detailed in Example 1.

FIG. 6 shows the result of a CDC assay where T cells expressing anti-CD123 CAR of SEQ ID NO 1-4 or control T Cell not expressing any anti CD123 CAR (mock T-cell (transfection step without any mRNA)) are incubated with Rituximab (RTX) and Baby Rabbit Complement BRC). The results are expressed as relative frequency of viable cells among anti-CD123 CAR positive T cells (with respect to control experiment) as detailed in Example 3.

FIGS. 7A, 7B and 7C discloses the general structure of CARs of SEQ ID NO 125 to 141 that have been designed and tested in Examples 4 to 6. An anti BCMA ScFV was used in all these CARs. One, two, three or four epitopes selected from a CD20 mimotope (black box named “mimotope”) and or a CD34 epitope (gray box named “CD34”) were included at different positions, i.e upstream to the ScFv, downstream to the ScFv or between the variable chains of the ScFv (noted as V1 and V2). As depicted in the FIG. 7, all the CARs have the same hinge (CD8 hinge), transmembrane domain (CD8 TM), co-stimulatory domain (4-1BB) and stimulatory domain (ITAM CD3 zeta).

FIGS. 8A and 8B shows the result of a CDC assay where T cells expressing anti-BCMA CAR of SEQ ID NO 125 or 130 to 141 are incubated with RTX and BRC. The results are expressed as relative frequency of viable cells among anti-BCMA CAR positive T cells (with respect to control experiment) as detailed in Example 4.

FIG. 9 shows the cytolytic activity of T cells expressing anti-BCMA CAR of SEQ ID NO 125 or 130 to 139 or control T Cell not expressing any anti BCMA CAR (T-cell). The cytolytic activity is expressed as the frequency of viable H929 cells as detailed in Example 4.

FIG. 10A shows the frequency of T-cells expressing CAR of SEQ ID NO 128 comprising a CD34 epitope and two CD20 mimotopes before or after purification with CD34 MicroBead Kit or in the flow through fraction.

FIG. 10B shows the number of T-cells expressing CAR of SEQ ID NO 128 comprising a CD34 epitope before or after purification with CD34 MicroBead Kit or in the flow through fraction.

FIG. 11 shows concentration of INF gamma produced by T cell expressing anti-BCMA CAR of SEQ ID NO 125 or 130 to 139 in the presence of RTX, phytohemagglutinin (PHA) or in the absence of both RTX anf PHA.

FIG. 12 shows the frequency of CAR positive T-Cell resulting from the detection of T Cell expressing CARs of SEQ ID NO 125 or 130 to 139 using a BCMA-Fc fusion protein and a labeled anti Fc antibody or RTX and a labeled anti Fc antibody.

FIG. 13 shows the frequency of CAR positive T-cells resulting from the detection of T Cell expressing CARs of SEQ ID NO 128 using RTX and a labeled anti Fc antibody or labeled QBEND-10.

FIGS. 14A and 14B shows the detection of T Cells expressing BCMA CARs of SEQ ID NO 145 (BC30, Wild type), 146 (LM), 147 (LML), 148 (LMLM and-149 (LMLML) containing CD20 mimotopes by flow cytometry. The CAR T cells are detected by flow cytometry using either soluble biotinylated-BCMA protein followed by PE-conjugated streptavidin (sBCMA biotin (PE) or the anti-CD20 antibody rituximab followed by FITC-conjugated anti-human IgG (Rituximab (FITC)).

FIG. 15 shows the detection of T cells not transduced, transduced with a lentivirus for the coexpression of an anti BCMA CAR of SEQ ID NO 145 and RQR8 (SEQ ID NO 150) (BC30-RQR8), or BCMA CARs of SEQ ID NO 149 containing CD20 mimotopes by flow cytometry. The CAR-T cells are detected by flow cytometry using either soluble biotinylated-BCMA protein followed by PE-conjugated streptavidin (sBCMA biotin (PE)) or the anti-CD20 antibody rituximab followed by FITC-conjugated anti-human IgG (Rituximab (FITC)).

FIG. 16 shows the result of a CDC assay where T cells expressing anti-BCMA CAR of SEQ ID NO 149 (BC30-R2), anti BCMA CAR of SEQ ID NO 145 or coexpressing an anti BCMA CAR of SEQ ID NO 145 and RQR8 (SEQ ID NO 150) (BC30-RQR8) are incubated with RTX and BRC. The percentage of cytotoxicity is determined by flow cytometry analysis using biotinylated BCMA protein. The results are expressed as the frequency of cell lysis among anti-BCMA CAR positive T cells with respect to control (cells incubated with BRC only).

FIG. 17 shows the cytolytic activity of T cells expressing anti-BCMA CAR of SEQ ID NO 149 (BC30-R2) or coexpressing an anti BCMA CAR of SEQ ID NO 145 (BC30) and RQR8 (SEQ ID NO 150) (BC30-RQR8) in the presence/absence of RTX. The cytolytic activity is expressed as the percentage of cell lysis calculated as disclosed in example 7.5 and is determined at different ratio of effector (CAR T cell): target (MM1S cells expressing BCMA).

FIG. 18 shows the percentage of activated T cells expressing anti-BCMA CAR of SEQ ID NO 149 in the presence of PBS (control), anti-CD3 OKT3 antibody (αCD3), or Rituximab (RTX). T cell activation is assessed by measuring the expression of the activation markers CD25 and CD69 using flow cytometry.

Table 1: Listing of the pharmaceutically-approved mAb with their antigenic targets. The sequences of the latter are provided, as well as epitope(s) for some of them.

Table 2: Listing of several mimotopes and epitopes corresponding to their mAb which are presented in Example 2.

Table 3: Listing of the VH & VL chains of scFv targeting the CD19, CD33, 5T4, ROR1, EGFRVIII, BCMA, CS1 and CD123 antigens.

Table 4: Exemplary sequence of CAR components

### DETAILED DESCRIPTION OF THE INVENTION

The invention relates to a polypeptide encoding a chimeric antigen receptor (CAR) comprising at least one extracellular binding domain that comprises a scFv formed by at least a VH chain and a VL chain specific to an antigen, preferably a cell surface marker antigen, wherein said extracellular binding domain comprises at least one mAb-specific epitope. In one embodiment, the mAb-specific epitope is an epitope to be bound by an epitope-specific mAb for in vitro cell sorting and/or in vivo cell depletion of T cells expressing a CAR comprising such epitope.

The invention relates to a polypeptide encoding a chimeric antigen receptor (CAR) comprising at least one extracellular binding domain that comprises a scFv formed by at least a VH chain and a VL chain specific to a cell surface marker antigen, wherein said extracellular binding domain comprises at least one mAb-specific epitope to be bound by a epitope-specific mAb for in vitro cell sorting and/or in vivo cell depletion of T cells expressing said CAR.

In some embodiments, the invention relates to a CAR comprising
an extracellular domain comprising
   at least one, preferably one, extracellular binding domain that comprises a scFv formed by at least a VH chain and a VL chain specific to an antigen, preferably a cell surface marker antigen, wherein said extracellular binding domain comprises at least one mAb-specific epitope, and,
   a hinge,
a transmembrane domain, and,
an intracellular domain.

In some embodiments, the invention relates to a CAR comprising
an extracellular domain comprising
   at least one extracellular binding domain that comprises a scFv formed by at least a VH chain and a VL chain specific to a cell surface marker antigen, wherein said extracellular binding domain comprises at least one mAb-specific epitope to be bound by a epitope-specific mAb for in vitro cell sorting and/or in vivo cell depletion of T cells expressing said CAR, and,
   a hinge,
a transmembrane domain, and,
an intracellular domain.

In embodiments, the CAR of the invention comprises one extracellular binding domain.

By “chimeric scFv” is meant a polypeptide corresponding to a single-chain variable fragment composed of heavy and light chains (V_{H} and V_{L}, respectively) and of an epitope, which was not originally included in said V_{H} and V_{L} chains. The latter epitope is referred to as “mAb-specific epitope” when it has the capacity to be bound specifically by an antibody, in particular a monoclonal antibody. In some embodiments, the mAbs specific epitope is not an epitope recognized by the ScFv. In some embodiments, the mAbs specific epitope is not derived from the extracellular domain of the CAR. The components of this chimeric scFv (i.e. the light and heavy variable fragments of the ligand binding domain and the mAb specific epitope) may be joined together by at least one linker, usually a flexible linker. These components are generally joined to the transmembrane domain of the CAR by a hinge.

Chimeric scFv Conformations

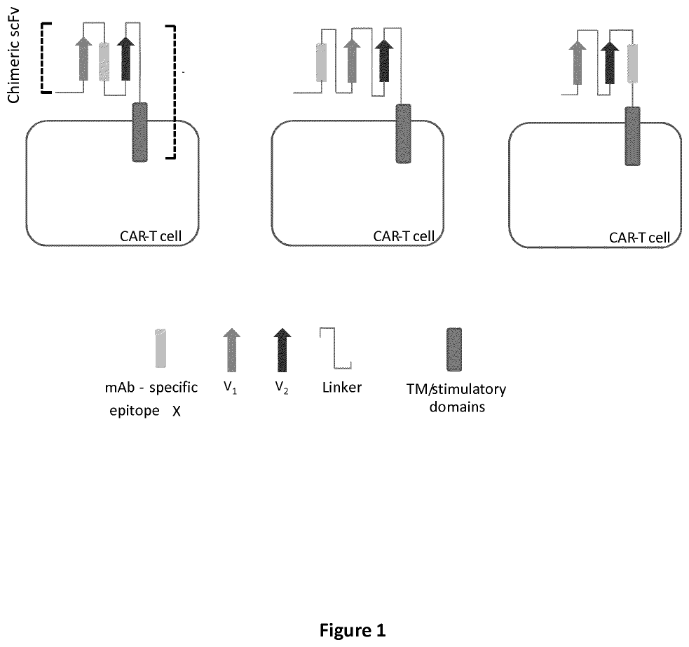

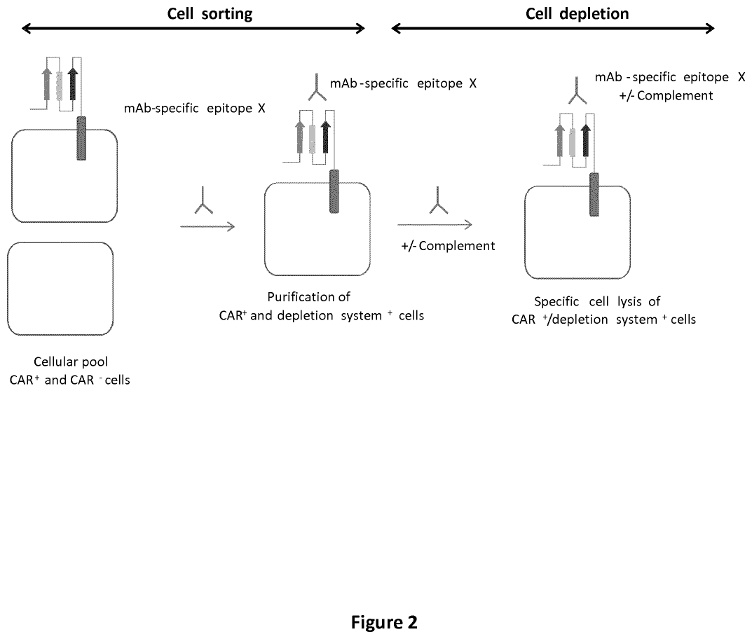

The structure of the chimeric scFv of the invention can be various as presented in the FIG. 1 depending of the position of its main components (V_{H} and V_{L} and m-Ab specific epitope).

The chimeric scFv of the invention may have several conformations, at least 9 when considering the number of possible permutations of one V_{H}, one V_{L} and one epitope.

Preferably, each component (VH, VL and epitope) is interconnected with its neighbor(s) by at least one flexible linker such as presented previously. The suitable combinations according to the invention are the ones which provide a good affinity/specificity in both bindings: between the mAb-specific epitope and the infused mAb, and between the VH & VL chains of the chimeric scFv and the antigen of the cell target ligand.

According to one embodiment, the extracellular-binding domain of the CAR comprises at least two linkers, both of them joining the epitope to the VH and VL chains; and a hinge joining the scFv-epitope to the transmembrane domain of the CAR.

For instance, if the projected CAR conformation is such that the mAb-specific epitope is located beside the VH and VL chains, a screening is performed when the CAR is expressed and is tested for cytoxicity and/or mAb depletion.

When the mAb-specific epitope is sought for being located between the VH and VL chains, a screening may be performed by phage display before testing and/or transient expression of the CAR construct. This may be obtained by transfection of mRNA, which is sufficient for a primary cytoxicity and/or mAb depletion test.

In some embodiments, the extracellular binding domain comprises at least 1, 2, 3, 4, 5, 6, 7, 8, 9 or 10 mAb-specific epitopes.

In some embodiments, the extracellular binding domain comprises 1, 2, 3, 4, 5, 6, 7, 8, 9 or 10 mAb-specific epitopes.

In some embodiments, the extracellular binding domain comprises 1, 2 or 3 mAb-specific epitopes.

In some embodiments, when the extracellular binding domain comprises several mAb-specific epitopes, all the mAb-specific epitopes are identical.

In some embodiments, when the extracellular binding domain comprises several mAb-specific epitopes, the mAb-specific epitopes are not identical. For example, the extracellular binding domain can comprises three mAb-specific epitopes, two of them being identical and the third one being different.

In some embodiments, the extracellular binding domain comprises a VH, a VL, and one or more mAb-specific epitopes, preferably 1, 2 or 3, more preferably 2 or 3 mAb-specific epitopes.

In some embodiments, the extracellular binding domain comprises the following sequence (Nterm is located on the left hand side):
V₁-L₁-V₂-(L)ₓ-Epitope1-(L)ₓ; V₁-L₁-V₂-(L)ₓ-Epitope1-(L)ₓ-Epitope2-(L)ₓ; V₁-L₁-V₂-(L)ₓ-Epitope1- (L)ₓ-Epitope2-(L)ₓ-Epitope3-(L)ₓ; (L)ₓ-Epitope1-(L)ₓ-V₁-L₁-V₂; (L)ₓ-Epitope1-(L)ₓ-Epitope2-(L)ₓ-V₁-L₁-V₂; Epitope1-(L)ₓ-Epitope2-(L)ₓ-Epitope3-(L)ₓ-V₁-L₁-V₂; (L)ₓ-Epitope1-(L)ₓ-V₁-L₁-V₂-(L)ₓ-Epitope2-(L)ₓ; (L)ₓ-Epitope1-(L)ₓ-V₁-L₁-V₂-(L)ₓ-Epitope2-(L)ₓ-Epitope3-(L)ₓ; (L)ₓ-Epitope1-(L)ₓ-V₁-L₁-V₂-(L)ₓ-Epitope2-(L)ₓ-(L)ₓ-Epitope1-(L)ₓ-Epitope2-(L)ₓ-V₁-L₁-V₂-(L)ₓ-Epitope3-(L)ₓ; (L)ₓ-Epitope3-(L)ₓ-Epitope4-(L)ₓ; Epitope1-(L)ₓ-Epitope2-(L)ₓ-V₁-L₁-V₂-(L)ₓ-Epitope3-(L)ₓ-Epitope4-(L)ₓ; V₁-(L)ₓ-Epitope1-(L)ₓ-V₂; V₁-(L)ₓ-Epitope1-(L)ₓ-V₂-(L)ₓ-Epitope2-(L)ₓ; V₁-(L)ₓ-Epitope1-(L)ₓ-V₂-(L)ₓ-Epitope2-(L)ₓ-Epitope3-(L)ₓ; V₁-(L)ₓ-Epitope1-(L)ₓ-V₂-(L)ₓ-Epitope2-(L)ₓ-Epitope3-(L)ₓ-Epitope4-(L)ₓ; (L)ₓ-Epitope1-(L)ₓ-V₁-(L)ₓ-Epitope2-(L)ₓ-V₂; (L)ₓ-Epitope1-(L)ₓ-V₁-(L)ₓ-Epitope2-(L)ₓ-V₂-(L)ₓ-Epitope3-(L)ₓ; V₁-L₁-V₂-L-Epitope1; V₁-L₁-V₂-L-Epitope1-L; V₁-L₁-V₂-L-Epitope1-L-Epitope2; V₁-L₁-V₂-L-Epitope1-L-Epitope2-L; V₁-L₁-V₂-L-Epitope1-L-Epitope2-L-Epitope3; V₁-L₁-V₂-L-Epitope1-L-Epitope2-L-Epitope3-L; V₁-L₁-V₂-Epitope1; V₁-L₁-V₂-Epitope1-L; V₁-L₁-V₂-Epitope1-L-Epitope2; V₁-L₁-V₂-Epitope1-L-Epitope2-L; V₁-L₁-V₂-Epitope1-L-Epitope2-L-Epitope3; V₁-L₁-V₂-Epitope1-L-Epitope2-L-Epitope3-L; Epitope1-V₁-L₁-V₂; Epitope1-L-V₁-L₁-V₂; L-Epitope1-V₁-L₁-V₂; L-Epitope1-L-V₁-L₁-V₂; Epitope1-L-Epitope2-V₁-L₁-V₂; Epitope1-L-Epitope2-L-V₁-L₁-V₂; L-Epitope1-L-Epitope2-V₁-L₁-V₂; L-Epitope1-L-Epitope2-L-V₁-L₁-V₂; Epitope1-L-Epitope2-L-Epitope3-V₁-L₁-V₂; Epitope1-L-Epitope2-L-Epitope3-L-V₁-L₁-V₂; L-Epitope1-L-Epitope2-L-Epitope3-V₁-L₁-V₂; L-Epitope1-L-Epitope2-L-Epitope3-L-V₁-L₁-V₂; V₁-L-Epitope1-L-V₂; L-Epitope1-L-V₁-L-Epitope2-L-V₂; V₁-L-Epitope1-L-V₂-L-Epitope2-L; V₁-L-Epitope1-L-V₂-L-Epitope2-L-Epitope3; V₁-L-Epitope1-L-V₂-L-Epitope2-Epitope 3; V₁-L-Epitope1-L-V₂-L-Epitope2-L-Epitope3-Epitope4; L-Epitope1-L-V₁-L-Epitope2-L-V₂-L-Epitope3-L; Epitope 1-L-V₁-L-Epitope2-L-V₂-L-Epitope3-L; L-Epitope 1-L-V₁-L-Epitope2-L-V₂-L-Epitope3; L-Epitope1-L-V₁-L₁-V₂-L-Epitope2-L; L-Epitope1-L-V₁-L₁-V₂-L-Epitope2-L-Epitope3; L-Epitope1-L-V₁-L₁-V₂-L-Epitope2-Epitope3, or Epitope1-L-V₁-L₁-V₂-L-Epitope2-L-Epitope3-Epitope4.
wherein,
V₁ and V₂ are V_{H} and V_{L} of an ScFv (i.e, V₁ is V₁ and V₂ is V_{H} or V₁ is V_{H} and V₂ is V_{L});
L₁ is any linker suitable to link the V_{H} chain to the V_{L} chain in an ScFv;
L is a linker, preferably comprising glycine and serine residues, and each occurrence of L in the extracellular binding domain can be identical or different to other occurrence of L in the same extracellular binding domain, and,
x is 0 or 1 and each occurrence of x is independently from the others; and, Epitope 1, Epitope 2 and Epitope 3 are mAb-specific epitopes and can be identical or different.

In some embodiments, the extracellular binding domains comprises the following sequence (Nterm is located on the left hand side):
V_{H}-L₁-VL-L-Epitope1-L-Epitope2-L; L-Epitope1-L-V_{H}-L-Epitope2-L-VL-L-Epitope3-L; V_{L}-L¹-V_{H}-L-Epitope1-L-Epitope2-L; or, L-Epitope1-L-VL-L-Epitope2-L-V_{H}-L-Epitope3-L,
wherein L, L₁, Epitope1, Epitope2 and Epitope3 are as defined above.

In some embodiments, L₁ is a linker comprising Glycine and/or Serine. In some embodiments, L₁ is a linker comprising the amino acid sequence (Gly-Gly-Gly-Ser)ₙ or (Gly-Gly-Gly-Gly-Ser)ₙ, where n is 1, 2, 3, 4 or 5. In some embodiments L₁ is (Gly₄Ser)₄ or (Gly₄Ser)₃.

In some embodiments, L is a flexible linker, preferably comprising Glycine and/or Serine. In some embodiments, L has an amino acid sequence selected from SGG, GGS, SGGS, SSGGS, GGGG, SGGGG, GGGGS, SGGGGS, GGGGGS, SGGGGGS, SGGGGG, GSGGGGS, GGGGGGGS, SGGGGGGG, SGGGGGGGS, or SGGGGSGGGGS preferably SGG, SGGS, SSGGS, GGGG, SGGGGS, SGGGGGS, SGGGGG, GSGGGGS or SGGGGSGGGGS. In some embodiments, when the extracellular binding domain comprises several occurrences of L, all the Ls are identical. In some embodiments, when the extracellular binding domain comprises several occurrences of L, the Ls are not all identical. In some embodiments, L is SGGGGS. In some embodiments, the extracellular binding domain comprises several occurrences of L and all the Ls are SGGGGS.

In some embodiments, Epitope 1, Epitope 2 and Epitope 3 are identical or different and are selected from mAb-specific epitopes having an amino acid sequence of SEQ ID NO 35, SEQ ID NO 36, SEQ ID NO 37, SEQ ID NO 38, SEQ ID NO 39, SEQ ID NO 40, SEQ ID NO 41 or SEQ ID NO 42, SEQ ID NO 144 or SEQ ID NO 174.

In some embodiments, Epitope 1, Epitope 2 and Epitope 3 are identical or different and are selected from mAb-specific epitopes specifically recognized by ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.

In some embodiments, Epitope 1 is a mAb-specific epitope having an amino acid sequence of SEQ ID NO 35.

In some embodiments, Epitope 2 is a mAb-specific epitope having an amino acid sequence of SEQ ID NO 35.

In some embodiments, Epitope 3 is a mAb-specific epitope having an amino acid sequence of SEQ ID NO 35.

In some embodiments, Epitope 4 is a mAb-specific epitope having an amino acid sequence of SEQ ID NO 35.

In some embodiments, Epitope 2 is a mAb-specific epitope having an amino acid sequence of SEQ ID NO 35 and Epitope 3 is an mAb-specific epitope having an amino acid sequence of SEQ ID NO 144.

In some embodiments, one of Epitope 1, Epitope 2, Epitope 3 and Epitope 4 is a CD34 epitope, preferably an epitope of SEQ ID 144. In some embodiments, one of Epitope1, Epitope 2, Epitope 3 and Epitope 4 is a CD34 epitope, preferably an epitope of SEQ ID 144 and the other mAb specific epitopes are CD20 mimotopes, preferably mimotope of SEQ ID NO 35.

Inserted mAb-Specific Epitope

According to the invention, the epitope to be inserted within the chimeric scFv is specific to the monoclonal antibody (mAb) which is used for cell sorting and/or cell depletion processes.

In a preferred embodiment, the introduced epitope within chimeric scFv is chosen as part of a mAb-specific epitope/epitope-specific mAb couple, in the basis of their approval by National Health Agencies in terms of regulatory/safety. Such couples are presented in the following table 1.

TABLE 1
Listing of pharmaceutically-approved monoclonal antibodies with their antigenic targets.

| | | | | |
|---|---|---|---|---|
| | | Drug bank accession | | |
| | | n° (or other n° if | Target/ | SEQ ID |
| Antibody | Indication | stated) | Antigen | NO |

Murine

| | | | | |
|---|---|---|---|---|
| Ibritumomab | Non-Hodgkin lymphoma | DB00078 | CD20 | SEQ ID |
| tiuxetan | (with yttrium-90 or | | | NO 11 |
| | indium-111) | | | |
| Muromonab-CD3 | Transplant rejection | DB00075 | T cell CD3 | SEQ ID |
| | | | Receptor | NO 12 |
| Tositumomab | Non-Hodgkin lymphoma | DB00081 | CD20 | SEQ ID |
| | | | | NO 11 |

Chimeric

| | | | | |
|---|---|---|---|---|
| Abciximab | Cardiovascular disease | DB00054 | inhibition of | SEQ ID |
| | | | glycoprotein | NO 13 |
| | | | IIb/IIIa | |
| Basiliximab | Transplant rejection | DB00074 | IL-2Rα receptor | SEQ ID |
| | | | (CD25) | NO 14 |
| Brentuximab | Anaplastic large cell | DB08870 | CD30 | SEQ ID |
| vedotin | lymphoma | | | NO 15 |
| Cetuximab | Colorectal cancer, Head | DB00002 | epidermal growth | SEQ ID |
| | and neck cancer | | factor receptor | NO 16 |
| Infliximab | Several autoimmune | DB00065 | inhibition of | SEQ ID |
| | disorders | | TNF-α signaling | NO 17 |
| Rituximab | Non-Hodgkin lymphoma | DB00073 | CD20 | SEQ ID |
| | | | | NO 11 |

Humanized

| | | | | |
|---|---|---|---|---|
| Alemtuzumab | Chronic lymphocytic | DB00087 | CD52 | SEQ ID |
| | leukemia | | | NO 18 |
| Bevacizumab | Colorectal cancer, Age | DB00112 | Vascular | SEQ ID |
| | related macular | | endothelial | NO 19 |
| | degeneration (off-label) | | growth factor | |
| | | | (VEGF) | |
| Certolizumab | Crohn's disease | DB08904 | inhibition of | SEQ ID |
| pegol | | | TNF-α signaling | NO 17 |
| Daclizumab | Transplant rejection | DB00111 | IL-2Rα receptor | SEQ ID |
| | | | (CD25) | NO 14 |
| Eculizumab | Paroxysmal nocturnal | DB01257 | Complement | SEQ ID |
| | hemoglobinuria | | system protein | NO 20 |
| Efalizumab | Psoriasis | DB00095 | CD11a | SEQ ID |
| | | | | NO 21 |
| Gemtuzumab | Acute myelogenous | DB00056 | CD33 | SEQ ID |
| | leukemia (with | | | NO 22 |
| | calicheamicin) | | | |
| Natalizumab | Multiple sclerosis and | DB00108 | alpha-4 (α4) | SEQ ID |
| | Crohn's disease | | integrin | NO 23 |
| Omalizumab | mainly allergy-related | DB00043 | immunoglobulin E | SEQ ID |
| | asthma | | (IgE) | NO 24 |
| Palivizumab | Respiratory Syncytial | DB00110 | an epitope of the | SEQ ID |
| | Virus | | RSV F protein | NO 25 |
| Ranibizumab | Macular degeneration | DB01270 | Vascular | SEQ ID |
| | | | endothelial | NO 19 |
| | | | growth factor A | |
| | | | (VEGF-A) | |
| Tocilizumab (or | Rheumatoid arthritis | DB06273 | Anti-IL-6R | SEQ ID |
| Atlizumab) | | | | NO 26 |
| Trastuzumab | Breast cancer | DB00072 | ErbB2 | SEQ ID |
| | | | | NO 27 |
| Vedolizumab | Crohn's disease, | CAS n°943609-66-3 | integrin α₄β₇ | SEQ ID |
| | ulcerative colitis | | | NO 28 |

Human

| | | | | |
|---|---|---|---|---|
| Adalimumab | Several auto-immune | DB00051 | inhibition of | SEQ ID |
| | disorders | | TNF-α signaling | NO 17 |
| Belimumab | Systemic lupus | DB08879 | inihibition of | SEQ ID |
| | erythematosus | | B-cell activating | NO 29 |
| | | | factor | |
| Canakinumab | Cryopyrin-associated | DB06168 | IL-1β | SEQ ID |
| | periodic syndrome | | | NO 30 |
| | (CAPS) | | | |
| Denosumab | Postmenopausal | DB06643 | RANK Ligand | SEQ ID |
| | osteoporosis, Solid | | inhibitor | NO 31 |
| | tumor's bony | | | |
| | metastases | | | |
| Golimumab | Rheumatoid arthritis, | DB06674 | TNF-alpha | SEQ ID |
| | Psoriatic arthritis, and | | inihibitor | NO 17 |
| | Ankylosing spondylitis | | | |
| Ipilimumab | Melanoma | DB06186 | blocks CTLA-4 | SEQ ID |
| (MDX-101) | | | | NO 32 |
| Ofatumumab | Chronic lymphocytic | CAS n° 679818-59-8 | CD20 | SEQ ID |
| | leukemia | | | NO 11 |
| Panitumumab | Colorectal cancer | DB01269 | epidermal growth | SEQ ID |
| | | | factor receptor | NO 16 |
| Ustekinumab | Psoriatic Arthritis, | DB05679 | IL-12, IL-23 | SEQ ID |
| | Plaque Psoriasis | | | NO 33 |
| Nivolumab | renal cell carcinoma, | CAS n°946414-94-4 | PD-1 | SEQ ID |
| | lung cancer, melanoma, | | | NO 34 |
| | and advanced or | | | |
| | metastatic solid tumors | | | |

TABLE 2
Examples of mAb-specific epitopes (and their
corresponding mAbs) that can be used in the
extracellular binding domain of the CAR of the
invention such as for example mimotopes and
epitope with their corresponding mAb as used in
the Examples 1-2
Rituximab

| | | |
|---|---|---|
| Mimotope | SEQ ID NO 35 | CPYSNPSLC |

Palivizumab

| | | |
|---|---|---|
| Epitope | SEQ ID NO 36 | NSELLSLINDMPITNDQKKLMSNN |

Cetuximab

| | | |
|---|---|---|
| Mimotope 1 | SEQ ID NO 37 | CQFDLSTRRLKC |
| Mimotope 2 | SEQ ID NO 38 | CQYNLSSRALKC |
| Mimotope 3 | SEQ ID NO 39 | CVWQRWQKSYVC |
| Mimotope 4 | SEQ ID NO 40 | CMWDRFSRWYKC |

Nivolumab

| | | |
|---|---|---|
| Epitope 1 | SEQ ID NO 41 | SFVLNWYRMSPSNQTDKLAAFPEDR |
| Epitope 2 | SEQ ID NO 42 | SGTYLCGAISLAPKAQIKE |

QBEND-10

| | | |
|---|---|---|
| Epitope | SEQ ID NO 144 | ELPTQGTFSNVSTNVSPAKPTTTA |

Alemtuzumab

| | | |
|---|---|---|
| Epitope | SEQ ID NO 174 | GQNDTSQTSSPS |

In a preferred embodiment, the epitope introduced within the chimeric scFv is the CD20 antigen, preferably SEQ ID NO 35 and the infused mAb which is being used to target it—for sorting and/or depletion purpose(s) is rituximab.

In some embodiments, the mAb-specific epitope has an amino acid sequence of SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 144 or SEQ ID NO 174.

In some embodiments, the extracellular binding domain of the CAR of the invention comprises one mAb-specific epitope of SEQ ID NO 35, two mAb-specific epitopes of SEQ ID NO 35, three mAb-specific epitopes of SEQ ID NO 35, one mAb-specific epitope of SEQ ID NO 35 and one mAb-specific epitope of SEQ ID NO 144, two mAb-specific epitopes of SEQ ID NO 35 and one mAb-specific epitope of SEQ ID NO 144, three mAb-specific epitopes of SEQ ID NO 35 and one mAb-specific epitope of SEQ ID NO 144.

According to another embodiment, the epitope is a mimotope. As a macromolecule, often a peptide, which mimics the structure of an epitope, the mimotope has the advantage to be smaller than conventional epitope, and therefore may be beneficial for a non-conformational sequence and easier to reproduce in a long polypeptide such a CAR. Mimotopes are known for several pharmaceutically-approved mAb such as two 10 amino acid peptides for cetuximab (Riemer et al., 2005), or a 24 aa for palivizumab (Arbiza et al, 1992). As these mimotopes can be identified by phage display, it is possible to try several of them in order to obtain a sequence which does not perturb the scFv for the same mAb. Furthermore, their use can enhance a complement-dependent cytotoxicity (CDC).

scFv

The term “extracellular ligand-binding domain” as used herein is defined as an oligo- or polypeptide that is capable of binding a ligand. Preferably, said domain is sought for being capable of interacting with a cell surface molecule. For example, the extracellular ligand-binding domain may be chosen to recognize a ligand that acts as a cell surface marker on target cells associated with a particular disease state. Thus examples of cell surface markers that may act as ligands include those associated with viral, bacterial and parasitic infections, autoimmune disease and cancer cells. In particular, the extracellular ligand-binding domain can comprise an antigen binding domain derived from an antibody against an antigen of the target. As non-limiting examples, the antigen of the target can be a tumor-associated surface antigen as described above. In some embodiments, the extracellular binding domain is an extracellular ligand-binding domain as defined above. According to the present invention, said extracellular ligand-binding domain is a single chain antibody fragment (scFv) comprising the light (V_{L}) and the heavy (V_{H}) variable fragment of a target antigen specific monoclonal antibody, and an mAb epitope specific antigen. In some embodiments, the extracellular binding domain comprises a single chain antibody fragment (scFv) comprising the light (V_{L}) and the heavy (V_{H}) variable fragment of a cell surface target antigen specific monoclonal antibody.

Other binding domain than scFv can also be used for predefined targeting of lymphocytes, such as camelid single-domain antibody fragments, receptor ligands like a vascular endothelial growth factor polypeptide, an integrin-binding peptide, heregulin or an IL-13 mutein, antibody binding domains, antibody hypervariable loops or CDRs as non-limiting examples.

In another embodiment, said extracellular binding domain can be a DARPin (designed ankyrin repeat protein). DARPins are genetically engineered antibody mimetic proteins typically exhibiting highly specific and high-affinity target protein binding. They are derived from natural ankyrin proteins and comprise at least three, usually four or five repeat motifs of these proteins. DARPins are small, single domain proteins which can be selected to bind any given target protein with high affinity and specificity (Epa, Dolezal et al. 2013; Friedrich, Hanauer et al. 2013; Jost, Schilling et al. 2013). According to the present invention, DARPins can be engineered to comprise multiple antigen recognition sites. Thus, said DARPins can be used to recognize a series of consecutive different antigens as well as a unique antigen. Thus, the present invention relates to a method comprising providing an immune cell, and expressing at the surface of said immune cell chimeric antigen receptor which comprises a designed ankyrin repeat protein capable of recognizing at least one specific ligand, preferably at two specific ligands.

As non-limiting example, the ligand of the target or the antigen recognized by the extracellular binding domain, preferably by the ScFv, can be a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, GD3, C-type lectin-like molecule-1 (CLL-1), ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap), LRP6, melamona-associated Chondroitin Sulfate Proteoglycan (MCSP), CD38/CS1, MART1, WT1, MUC1, LMP2, Idiotype, NY-ESO-1, Ras mutant, gp100, proteinase 3, bcr-abl, tyrosinase, hTERT, EphA2, ML-TAP, ERG, NA17, PAX3, ALK, Androgen receptor; a lineage-specific or tissue specific antigen such as CD3, CD4, CD8, CD24, CD25, CD33, CD34, CD70, CD79, CD116, CD117, CD135, CD123, CD133, CD138, CTLA-4, B7-1 (CD80), B7-2 (CD86), endoglin, a major histocompatibility complex (MHC) molecule, BCMA (CD269, TNFRSF 17), FLT-3, or a virus-specific surface antigen such as an HIV-specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface markers. In specific cases, the ligand that the chimeric antigen receptor recognizes is present on the surface of a target cell, particularly cancer cell or viral cell. In some embodiments, the ligand that the chimeric antigen receptor recognizes is present in a tumor microenvironment. In some aspects of the invention, the ligand that the chimeric antigen receptor recognizes is a growth factor.

In one preferred embodiment, said VH and VL chains have as antigenic target sequence of over 80% identity, preferably over 90%, and more preferably over 95% with SEQ ID NO 43 (CD19 antigen), SEQ ID NO 44 (CD38 antigen), SEQ ID NO 45 (CD123 antigen), SEQ ID NO 46 (CS1 antigen), SEQ ID NO 47 (BCMA antigen), SEQ ID NO 48 (FLT-3 antigen), SEQ ID NO 49 (CD33 antigen), SEQ ID NO 50 (CD70 antigen), SEQ ID NO 51 (EGFR-3v antigen), SEQ ID NO 52 (WT1 antigen).

In one more preferred embodiment, said VH and VL chains have as antigenic target sequence of over 80% identity, preferably over 90%, and more preferably over 95% with or identical to SEQ ID NO 53-64 (CD19 antigen), SEQ ID NO 65-76 (CD33 antigen), SEQ ID NO 77-84 (5T4 antigen), SEQ ID NO 85-90 (ROR1 antigen), SEQ ID NO 91-94 (EGFRvIII antigen), SEQ ID NO 95-102 (BCMA antigen), SEQ ID NO 103-112 (CS1 antigen) and SEQ ID NO 113-124 (CD123 antigen) as follows in Table 3.

In some embodiments, the antigen recognized by the extracellular binding domain, preferably by the ScFv is selected from SEQ ID NO 43 (CD19 antigen), SEQ ID NO 44 (CD38 antigen), SEQ ID NO 45 (CD123 antigen), SEQ ID NO 46 (CS1 antigen), SEQ ID NO 47 (BCMA antigen), SEQ ID NO 48 (FLT-3 antigen), SEQ ID NO 49 (CD33 antigen), SEQ ID NO 50 (CD70 antigen), SEQ ID NO 51 (EGFR-vIII antigen) or SEQ ID NO 52 (WT1 antigen).

In some embodiments, the extracellular binding domain comprises:
a VH of SEQ ID NO 65 and a VL of SEQ ID NO 66; a VH of SEQ ID NO 67 and a VL of SEQ ID NO 68;
a VH of SEQ ID NO 69 and a VL of SEQ ID NO 70; a VH of SEQ ID NO 71 and a VL of SEQ ID NO 72; a VH of SEQ ID NO 77 and a VL of SEQ ID NO 78; a VH of SEQ ID NO 79 and a VL of SEQ ID NO 80;
a VH of SEQ ID NO 81 and a VL of SEQ ID NO 82; a VH of SEQ ID NO 83 and a VL of SEQ ID NO 84;
a VH of SEQ ID NO 85 and a VL of SEQ ID NO 86; a VH of SEQ ID NO 87 and a VL of SEQ ID NO 88; a VH of SEQ ID NO 89 and a VL of SEQ ID NO 90; a VH of SEQ ID NO 91 and a VL of SEQ ID NO 92; a VH of SEQ ID NO 93 and a VL of SEQ ID NO 94; a VH of SEQ ID NO 95 and a VL of SEQ ID NO 96; a VH of SEQ ID NO 97 and a VL of SEQ ID NO 98; a VH of SEQ ID NO 99 and a VL of SEQ ID NO 100; a VH of SEQ ID NO 101 and a VL of SEQ ID NO 102; a VH of SEQ ID NO 103 and a VL of SEQ ID NO 104; a VH of SEQ ID NO 105 and a VL of SEQ ID NO 106; a VH of SEQ ID NO 107 and a VL of SEQ ID NO 108; a VH of SEQ ID NO 109 and a VL of SEQ ID NO 110; a VH of SEQ ID NO 111 and a VL of SEQ ID NO 112; a VH of SEQ ID NO 113 and a VL of SEQ ID NO 114; a VH of SEQ ID NO 115 and a VL of SEQ ID NO 116; a VH of SEQ ID NO 117 and a VL of SEQ ID NO 118; a VH of SEQ ID NO 119 and a VL of SEQ ID NO 120; a VH of SEQ ID NO 121 and a VL of SEQ ID NO 122; a VH of SEQ ID NO 123 and a VL of SEQ ID NO 124; a VH of SEQ ID NO 170 and a VL of SEQ ID NO 171; a VH of SEQ ID NO 172 and a VL of SEQ ID NO 173; or, a VH of SEQ ID NO 174 and a VL of SEQ ID NO 175.

TABLE 3
Listing of the VH & VL chains of scFv targeting the CD19, CD33, 5T4, ROR1,
EGFRvIII, BCMA, CS1 and CD123 antigens

| | Name of | | |
|---|---|---|---|
| Cell | scFV & | SEQ | |
| surface | VH or VL | ID | |
| antigen | chain | NO | Polypeptide sequence |
| CD19 | CD19-1 | 53 | EVKLQESGPGLVAPSQSLSVTCTVSGVSLPDYGVSWIRQPPRKGLEWLGVIWG |
| | VH chain | | SETTYYNSALKSRLTIIKDNSKSQVFLKMNSLQTDDTAIYYCAKHYYYGGSYAM |
| | | | DYWGQGTSVTV |
| | CD19-1 | 54 | DIQMTQTTSSLSASLGDRVTISCRASQDISKYLNWYQQKPDGTVKLLIYHTSRL |
| | VL chain | | HSGVPSRFSGSGSGTDYSLTISNLEQEDIATYFCQQGNTLPYTFGGGTKLEIT |
| | CD19-2 | 55 | EVQLQQSGPELIKPGASVKMSCKASGYTFTSYVMHWVKQKPGQGLEWIGYI |
| | VH chain | | NPYNDGTKYNEKFKGKATLTSDKSSSTAYMELSSLTSEDSAVYYCARGTYYYGS |
| | | | RVFDYWGQGTTLTV |
| | CD19-2 | 56 | DIVMTQAAPSIPVTPGESVSISCRSSKSLLNSNGNTYLYWFLQRPGQSP |
| | VL chain | | QLLIYRMSNLASGVPDRFSGSGSGTAFTLRISRVEAEDVGVYYCMQHL |
| | | | EYPFTFGAGTKLELK |
| | 26292 VH | 57 | QVQLQQPGAELVRPGASVKLSCKASGYTFTSYWMNWVKQRPDQGLEWIGRI |
| | chain | | DPYDSETHYNQKFKDKAILIVDKSSSTAYMQLSSLTSEDSAVYYCARGNWDDY |
| | | | WGQGTTLTVSS |
| | 26292 VL | 58 | DVQITQSPSYLAASPGETITINCRASKSISKDLAWYQEKPGKTNKWYSGSTLQS |
| | chain | | GIPSRFSGSGSGTDFTLTISSLEPEDFAMYYCQQHNKYPYTFGGGTKLEIK |
| | | | QIQLVQSGPELKKPGETVKISCKASGYIFTNYGMNWVKQAPGKSFKWMGWI |
| | 32716 VH | 59 | NTYTGESTYSADFKGRFAFSLETSASTAYLHINDLKNEDTATYFCARSGGYDPM |
| | chain | | DYWGQGTSVTVSS |
| | 32716 VL | 60 | DIVLTQSPASLAVSLGQRATISCRASESVDNYGNTFMHWYQQKPGQPPKLLIY |
| | chain | | RASNLESGIPARFSGSGSRTDFTLTINPVEADDVATYYCQQSNEDPPTFGAGTK |
| | | | LELK |
| | Klon43 | 61 | MADYKDIVMTQSHKFMSTSVGDRVNITCKASQNVDSAVAWYQQKPGQSPK |
| | VH chain | | ALIYSASYRYSGVPDRFTGRGSGTDFTLTISSVQAEDLAVYYCQQYYSTPWTFG |
| | | | GGTKLEIKR |
| | Klon43 VL | 62 | EVKLVESGGGLVQPGGSLSLSCAASGFTFTDYYMSWVRQPPGKALEWLALIRS |
| | chain | | KADGYTTEYSASVKGRFTLSRDDSQSILYLQMNALRPEDSATYYCARDAAYYSY |
| | | | YSPEGAMDYWGQGTSVTVSS |
| | 12F1 VH | 63 | VQLQESGPGLVKPSQSLSLTCSVTDYSITSGYYWNWIRQFPGNKLEWMGYISY |
| | chain | | DGSNNYNPSLKNRISITRDTSKNQFFLKLSSVTTEDTATYYCSRGEGFYFDSWG |
| | | | QGTTLTVSSARS |
| | 12F1 VL | 64 | DIMMSQSPSSLAVSVGEKFTMTCKSSQSLFFGSTQKNYLAWYQQKPGQSPKL |
| | chain | | LIYWASTRESGVPDRFTGSGSGTDFTLAISSVMPEDLAVYYCQQYYNYPWTFG |
| | | | GGTKLEIK |
| CD33 | M195 VH | 65 | EVQLQQSGPELVKPGASVKISCKASGYTFTDYNMHWVKQSHGKSLEWIGYIY |
| | chain | | PYNGGTGYNQKFKSKATLTVDNSSSTAYMDVRSLTSEDSAVYYCARGRPAMD |
| | | | YWGQGTSVTVS |
| | M195 VL | 66 | DIVLTQSPASLAVSLGQRATISCRASESVDNYGISFMNWFQQKPGQPPKLLIYA |
| | chain | | ASNQGSGVPARFSGSGSGTDFSLNIHPMEEDDTAMYFCQQSKEVPWTFGGG |
| | | | TKLEIK |
| | m2H12 | 67 | QVQLQQSGPELVRPGTFVKISCKASGYTFTNYDINWVNQRPGQGLEWIGWIY |
| | VH chain | | PGDGSTKYNEKFKAKATLTADKSSSTAYLQLNNLTSENSAVYFCASGYEDAMD |
| | | | YWGQGTSVTVSS |
| | m2H12 VL | 68 | DIKMTQSPSSMYASLGERVIINCKASQDINSYLSWFQQKPGKSPKTLIYRANRL |
| | chain | | VDGVPSRFSGSGSGQDYSLTISSLEYEDMGIYYCLQYDEFPLTFGAGTKLELKR |
| | DRB2 VH | 69 | EVKLQESGPELVKPGASVKMSCKASGYKFTDYVVHWLKQKPGQGLEWIGYIN |
| | chain | | PYNDGTKYNEKFKGKATLTSDKSSSTAYMEVSSLTSEDSAVYYCARDYRYEVYG |
| | | | MDYWGQGTSVTVSS |
| | DRB2 VL | 70 | DIVLTQSPTIMSASPGERVTMTCTASSSVNYIHWYQQKSGDSPLRWIFDTSKV |
| | chain | | ASGVPARFSGSGSGTSYSLTISTMEAEDAATYYCQQWRSYPLTFGDGTRLELK |
| | | | RADAAPTVS |
| | My9-6 VH | 71 | QVQLQQPGAEVVKPGASVKMSCKASGYTFTSYYIHWIKQTPGQGLEWVGVIY |
| | chain | | PGNDDISYNQKFKGKATLTADKSSTTAYMQLSSLTSEDSAVYYCAREVRLRYFD |
| | | | VWGAGTTVTVSS |
| | My9-6 VL | 72 | NIMLTQSPSSLAVSAGEKVTMSCKSSQSVFFSSSQKNYLAWYQQIPGQSPKLLI |
| | chain | | YWASTRESGVPDRFTGSGSGTDFTLTISSVQSEDLAIYYCHQYLSSRTFGGGTKL |
| | | | EIKR |
| | M195 VH | 73 | EVQLQQSGPELVKPGASVKISCKASGYTFTDYNMHWVKQSHGKSLEWIGYIY |
| | chain | | PYNGGTGYNQKFKSKATLTVDNSSSTAYMDVRSLTSEDSAVYYCARGRPAMD |
| | | | YWGQGTSVTVS |
| | M195 VL | 74 | DIVLTQSPASLAVSLGQRATISCRASESVDNYGISFMNWFQQKPGQPPKLLIYA |
| | chain | | ASNQGSGVPARFSGSGSGTDFSLNIHPMEEDDTAMYFCQQSKEVPWTFGGG |
| | | | TKLEIK |
| | m2H12 | 75 | QVQLQQSGPELVRPGTFVKISCKASGYTFTNYDINWVNQRPGQGLEWIGWIY |
| | VH chain | | PGDGSTKYNEKFKAKATLTADKSSSTAYLQLNNLTSENSAVYFCASGYEDAMD |
| | | | YWGQGTSVTVSS |
| | m2H12 VL | 76 | DIKMTQSPSSMYASLGERVIINCKASQDINSYLSWFQQKPGKSPKTLIYRANRL |
| | chain | | VDGVPSRFSGSGSGQDYSLTISSLEYEDMGIYYCLQYDEFPLTFGAGTKLELKR |
| 5T4 | H8 heavy | 77 | EVQLQQSGPDLVKPGASVKISCKASGYSFTGYYMHWVKQSHGKSLEWIGRIN |
| | chain | | PNNGVTLYNQKFKDKAILTVDKSSTTAYMELRSLTSEDSAVYYCARSTMITNYV |
| | | | MDYWGQVTSVTVSS |
| | H8 VL | 78 | SIVMTQTPTFLLVSAGDRVTITCKASQSVSNDVAWYQQKPGQSPTLLISYTSSR |
| | chain | | YAGVPDRFIGSGYGTDFTFTISTLQAEDLAVYFCQQDYNSPPTFGGGTKLEIKR |
| | A3 heavy | 79 | QIQLVQSGPELKKPGETVKISCKASGYTFTNFGMNWVKQGPGEGLKWMGWI |
| | chain | | NTNTGEPRYAEEFKGRFAFSLETTASTAYLQINNLKNEDTATYFCARDWDGAY |
| | | | FFDYWGQGTTLTVSS |
| | A3 light | 80 | SIVMTQTPKFLLVSAGDRVTITCKASQSVSNDVAWYQQKPGQSPKLLINFATN |
| | chain | | RYTGVPNRFTGSGYGTDFTFTISTVQAEDLALYFCQQDYSSPWTFGGGTKLEIK |
| | A2 heavy | 81 | QVQLQQSRPELVKPGASVKMSCKASGYTFTDYVISWVKQRTGQGLEWIGEIY |
| | chain | | PGSNSIYYNEKFKGRATLTADKSSSTAYMQLSSLTSEDSAVYFCAMGGNYGFD |
| | | | YWGQGTTLTVSS |
| | A2 light | 82 | QIVLTQSPAIMSASLGERVTLTCTASSSVNSNYLHWYQQKPGSSPKLWIYSTSN |
| | chain | | LASGVPARFSGSGSGTSYSLTISSMEAEDAATYYCHQYHRSPLTFGAGTKLELK |
| | A3 heavy | 83 | EVQLVESGGGLVQPKGSLKLSCAASGFTFNTYAMNWVRQAPGKGLEWVARI |
| | chain | | RSKSNNYATYYADSVKDRFTISRDDSQSMLYLQMNNLKTEDTAMYYCVRQW |
| | | | DYDVRAMNYWGQGTSVTVSS |
| | A3 light | 84 | DIVMTQSHIFMSTSVGDRVSITCKASQDVDTAVAWYQQKPGQSPKLLIYWAS |
| | chain | | TRLTGVPDRFTGSGSGTDFTLTISNVQSEDLADYFCQQYSSYPYTFGGGTKLEIK |
| ROR1 | 2A2 | 85 | QVQLQQSGAELVRPGASVTLSCKASGYTFSDYEMHWVIQTPVHGLEWIGAID |
| | heavy | | PETGGTAYNQKFKGKAILTADKSSSTAYMELRSLTSEDSAVYYCTGYYDYDSFT |
| | chain | | YWGQGTLVTVSA |
| | 2A2 VL | 86 | DIVMTQSQKIMSTIVGDRVSITCKASQNVDAAVAWYQQKPGQSPKWYSAS |
| | chain | | NRYTGVPDRFTGSGSGTDFTLTISNMQSEDLADYFCQQYDIYPYTFGGGTKLEI |
| | | | K |
| | 4A5 | 87 | EVKLVESGGGLVKPGGSLKLSCAASGFTFSSYAMSWVRQIPEKRLEWVASISR |
| | heavy | | GGTTYYPDSVKGRFTISRDNVRNILYLQMSSLRSEDTAMYYCGRYDYDGYYAM |
| | chain | | DYWGQGTSVTVSS |
| | 4A5 light | 88 | DIKMTQSPSSMYASLGERVTITCKASPDINSYLSWFQQKPGKSPKTLIYRANRL |
| | hain | | VDGVPSRFSGGGSGQDYSLTINSLEYEDMGIYYCLQYDEFPYTFGGGTKLEMK |
| | D10 | 89 | QVQLKESGPGLVAPSQTLSITCTVSGFSLTSYGVHWVRQPPGKGLEWLGVIW |
| | heavy | | AGGFTNYNSALKSRLSISKDNSKSQVLLKMTSLQTDDTAMYYCARRGSSYSMD |
| | chain | | YWGQGTSVTVSS |
| | D10 light | 90 | EIVLSQSPAITAASLGQKVTITCSASSNVSYIHWYQQRSGTSPRPWIYEISKLASG |
| | chain | | VPVRFSGSGSGTSYSLTISSMEAEDAAIYYCQQWNYPLITFGSGTKLEIQ |
| EGFRvIII | 139- | 91 | EVQVLESGGGLVQPGGSLRLSCAASGFTFSSYAMSWVRQAPGKGLEWVSAIS |
| | heavy | | GSGGSTNYADSVKGRFTISRDNSKNTLYLQMNSLRAEDTAVYYCAGSSGWSE |
| | chain | | YWGQGTLVIVSS |
| | 139- | 92 | DIQMTQSPSSLSASVGDRVTITCRASQGIRNNLAWYQQKPGKAPKRLIYAASN |
| | VL chain | | LQSGVPSRFTGSGSGTEFTLIVSSLQPEDFATYYCLQHHSYPLTSGGGTKVEIK |
| | MR1- | 93 | QVQLQQSGGGLVKPGASLKLSCVTSGFTFRKFGMSWVRQTSDKRLEWVASIS |
| | heavy | | TGGYNTYYSDNVKGRFTISRENAKNTLYLQMSSLKSEDTALYYCTRGYSSTSYA |
| | chain | | MDYWGQGTTVTV |
| | MR1- | 94 | DIELTQSPASLSVATGEKVTIRCMTSTDIDDDMNWYQQKPGEPPKFLISEGNTL |
| | light | | RPGVPSRFSSSGTGTDFVFTIENTLSEDVGDYYCLQSFNVPLTFGDGTKLEKAL |
| | chain | | |
| BCMA | BCMA-50 | 95 | QVQLVQSGAEVKKPGASVKVSCKASGYSFPDYYINWVRQAPGQGLEWMGW |
| | VH chain | | IYFASGNSEYNQKFTGRVTMTRDTSINTAYMELSSLTSEDTAVYFCASLYDYDW |
| | | | YFDVWGQGTMVTVSS |
| | BCMA-50 | 96 | DIVMTQTPLSLSVTPGQPASISCKSSQSLVHSNGNTYLHWYLQKPGQSPQLLIY |
| | VL chain | | KVSNRFSGVPDRFSGSGSGTDFTLKISRVEAEDVGIYYCSQSSIYPWTFGQGTK |
| | | | LEIK |
| | BCMA-30 | 97 | QVQLVQSGAEVKKPGASVKVSCKASGYSFPDYYINWVRQAPGQGLEWMGW |
| | VH chain | | IYFASGNSEYNQKFTGRVTMTRDTSSSTAYMELSSLRSEDTAVYFCASLYDYD |
| | | | WYFDVWGQGTMVIVSS |
| | BCMA-30 | 98 | DIVMTQTPLSLSVTPGEPASISCKSSQSLVHSNGNTYLHWYLQKPGQSPQLLIY |
| | VL chain | | KVSNRFSGVPDRFSGSGSGADFTLKISRVEAEDVGVYYCAETSHVPWTFGQGT |
| | | | KLEIK |
| | C11D5.3 | 99 | QIQLVQSGPELKKPGETVKISCKASGYTFTDYSINWVKRAPGKGLKWMGWIN |
| | VH chain | | TETREPAYAYDFRGRFAFSLETSASTAYLQINNLKYEDTATYFCALDYSYAMDY |
| | | | WGQGTSVTVSS |
| | C11D5.3 | 100 | DIVLTGSPPSLAMSLGKRATISCRASESVTILGSHLIHWYQQKPGQPPTLLIQLA |
| | VL chain | | SNVQTGVPARFSGSGSRTDFTLTIDPVEEDDVAVYYCLQSRTIPRTFGGGTKLEI |
| | | | K |
| | C13F12.1 | 101 | QIQLVQSGPELKKPGETVKISCKASGYTFTHYSMNWVKQAPGKGLKWMGRI |
| | VH chain | | NTETGEPLYADDFKGRFAFSLETSASTAYLVINNLKNEDTATFFCSNDYLYSCDY |
| | | | WGRGTTLTVSS |
| | C13F12.1 | 102 | DIVLTQSPPSLAMSLGKRATISCRASESVTILGSHLIYWYQQKPGQPPTLLIQLAS |
| | VL chain | | NVQTGVPARFSGSGSRTDFTLTIDPVEEDDVAVYYCLQSRTIPRTFGGGTKLEIK |
| CS1 | Luc63 VH | 103 | EVKLLESGGGLVQPGGSLKLSCAASGFDFSRYWMSWVRQAPGKGLEWIGEIN |
| | chain | | PDSSTINYTPSLKDKFIISRDNAKNTLYLQMSKVRSEDTALYYCARPDGNYWYF |
| | | | DVWGAGTTVTVSS |
| | Luc63 VL | 104 | DIVMTQSHKFMSTSVGDRVSITCKASQDVGIAVAWYQQKPGQSPKWYWAS |
| | chain | | TRHTGVPDRFTGSGSGTDFTLTISNVQSEDLADYFCQQYSSYPYTFGGGTKLEI |
| | | | K |
| | Luc90 VH | 105 | QVQLQQPGAELVRPGASVKLSCKASGYSFTTYWMNWVKQRPGQGLEWIG |
| | chain | | MIHPSDSETRLNQKFKDKATLTVDKSSSTAYMQLSSPTSEDSAVYYCARSTMIA |
| | | | TRAMDYWGQGTSVTVSS |
| | Luc90 VL | 106 | DIVMTQSQKSMSTSVGDRVSITCKASQDVITGVAWYQQKPGQSPKLLIYSASY |
| | chain | | RYTGVPDRFTGSGSGTDFTFTISNVQAEDLAVYYCQQHYSTPLTFGAGTKLELK |
| | Luc34 VH | 107 | QVQLQQSGAELARPGASVKLSCKASGYTFTSYWMQWVKQRPGQGLEWIGA |
| | chain | | IYPGDGDTRYTQKFKGKATLTADKSSSTAYMQLSSLASEDSAVYYCARGKVYYG |
| | | | SNPFAYWGQGTLVTVSA |
| | Luc34 VL | 108 | DIQMTQSSSYLSVSLGGRVTITCKASDHINNWLAWYQQKPGNAPRLLISGATS |
| | chain | | LETGVPSRFSGSGSGKDYTLSITSLQTEDVATYYCQQYWSTPWTFGGGTKLEIK |
| | LucX1 VH | 109 | QVQLQQSGPELVKPGASVKISCKASGYAFSSSWMNWVKQRPGQGLEWIGRI |
| | chain | | YPGDGDTKYNGKFKGKATLTADKSSSTAYMQLSSLTSVDSAVYFCARSTMIAT |
| | | | GAMDYWGQGTSVTVSS |
| | LucX1 VL | 110 | ETTVTQSPASLSMAIGEKVTIRCITSTDIDDDMNWYQQKPGEPPKLLISEGNTL |
| | chain | | RPGVPSRFSSSGYGTDFVFTIENMLSEDVADYYCLQSDNLPLTFGGGTKLEIK |
| | LucX2 VH | 111 | QVQLQQSGPELVKPGASVKISCKASGYAFSSSWMNWVKQRPGQGLEWIGRI |
| | chain | | YPGDGDTKYNGKFKGKATLTADKSSSTAYMQLSSLTSVDSAVYFCARSTMIAT |
| | | | GAMDYWGQGTSVTVSS |
| | LucX2 VL | 112 | DIVMTQSHKFMSTSVGDRVSITCKASQDVSTAVAWYQQKPGQSPKWYSASY |
| | chain | | RYTGVPDRFTGSGSGTDFTFTISSVQAEDLAVYYCQQHYSTPPYTFGGGTKLEI |
| | | | K |
| CD123 | 7G3 VH | 113 | MGWSWIFLFLVSGTGGVLSEVQLQQSGPELVKPGASVKMSCKASGYTFTDYY |
| | chain | | MKWVKQSHGKSLEWIGDIIPSNGATFYNQKFKGKATLTVDRSSSTAYMHLNS |
| | | | LTSEDSAVYYCTRSHLLRASWFAYWGQGTLVTVSAAS |
| | 7G3 VL | 114 | MESQTQVLMSLLFWVSGTCGDFVMTQSPSSLTVTAGEKVTMSCKSSQSLLNS |
| | chain | | GNQKNYLTWYLQKPGQPPKLLIYWASTRESGVPDRFTGSGSGTDFTLTISSVQ |
| | | | AEDLAVYYCQNDYSYPYTFGGGTKLEIKR |
| | Old4 VH | 115 | WTWRFLFVVAAATGVQSQVQLLQSGAEVKKPGSSVKVSCKASGGTFSTYAIS |
| | chain | | WVRQAPGQGLEWMGGIIPIFGIVNYAQKFQGRVTITADESTSTAYMELSSLRS |
| | | | EDTAVYYCARGGGSGPDVLDIWGQGTMVTVSSAST |
| | Old4 VL | 116 | MDMRVPAQLLGLLLLWLPGARCVIWMTQSPSLLSASTGDRVTISCRMSQGIR |
| | chain | | SYLAWYQQKPGKAPELLIYAASTLQSGVPSRFSGSGSGTDFTLTISSLQSEDFAT |
| | | | YYCQQYYSFPYTFGQGTKLEIKRTV |
| | 26292 VH | 117 | QVQLQQPGAELVRPGASVKLSCKASGYTFTSYWMNWVKQRPDQGLEWIGRI |
| | chain | | DPYDSETHYNQKFKDKAILWDKSSSTAYMQLSSLTSEDSAVYYCARGNWDDY |
| | | | WGQGTTLTVSS |
| | 26292 VL | 118 | DVQITQSPSYLAASPGETITINCRASKSISKDLAWYQEKPGKTNKWYSGSTLQS |
| | chain | | GIPSRFSGSGSGTDFTLTISSLEPEDFAMYYCQQHNKYPYTFGGGTKLEIK |
| | 32716 VH | 119 | QIQLVQSGPELKKPGETVKISCKASGYIFTNYGMNWVKQAPGKSFKWMGWI |
| | chain | | NTYTGESTYSADFKGRFAFSLETSASTAYLHINDLKNEDTATYFCARSGGYDPM |
| | | | DYWGQGTSVTVSS |
| | 32716 VL | 120 | DIVLTQSPASLAVSLGQRATISCRASESVDNYGNTFMHWYQQKPGQPPKLLIY |
| | chain | | RASNLESGIPARFSGSGSRTDFTLTINPVEADDVATYYCQQSNEDPPTFGAGTK |
| | | | LELK |
| | Klon43 | 121 | EVKLVESGGGLVQPGGSLSLSCAASGFTFTDYYMSWVRQPPGKALEWLALIRS |
| | VH chain | | KADGYTTEYSASVKGRFTLSRDDSQSILYLQMNALRPEDSATYYCARDAAYYSY |
| | | | YSPEGAMDYWGQGTSVTVSS |
| | Klon43 VL | 122 | MADYKDIVMTQSHKFMSTSVGDRVNITCKASQNVDSAVAWYQQKPGQSPK |
| | chain | | ALIYSASYRYSGVPDRFTGRGSGTDFTLTISSVQAEDLAVYYCQQYYSTPWTFG |
| | | | GGTKLEIKR |
| | 12F1 VH | 123 | VQLQESGPGLVKPSQSLSLTCSVTDYSITSGYYWNWIRQFPGNKLEWMGYISY |
| | chain | | DGSNNYNPSLKNRISITRDTSKNQFFLKLSSVTTEDTATYYCSRGEGFYFDSWG |
| | | | QGTTLTVSSARS |
| | 12F1 VL | 124 | DIMMSQSPSSLAVSVGEKFTMTCKSSQSLFFGSTQKNYLAWYQQKPGQSPKL |
| | chain | | LIYWASTRESGVPDRFTGSGSGTDFTLAISSVMPEDLAVYYCQQYYNYPWTFG |
| | | | GGTKLEIK |
| FAP | Humanized | 170 | QVQLVQSGAEVKKPGASVKVSCKTSRYTFTEYTIHWVRQAPGQRLEWIGGIN |
| | F19 VH | | PNNGIPNYNQKFKGRVTITVDTSASTAYMELSSLRSEDTAVYYCARRRIAYGYD |
| | chain | | EGHAMDYWGQGTLVTVSS |
| | Humanized | 171 | DIVMTQSPDSLAVSLGERATINCKSSQSLLYSRNQKNYLAWYQQKPGQPPKLLI |
| | F19 VL | | FWASTRESGVPDRFSGSGFGTDFTLTISSLQAEDVAVYYCQQYFSYPLTFGQGT |
| | chain | | KVEIK |
| | FAP5 VH | 172 | QVQLQQSGAELARPGASVNLSCKASGYTFTNNGINWLKQRTGQGLEWIGEIY |
| | chain | | PRSTNTLYNEKFKGKATLTADRSSNTAYMETELRSLTSEDSAVYFCARTLTAPFA |
| | | | FWGQGTLVTVSA |
| | FAP5 VL | 173 | QIVLTQSPAIMSASPGEKVTMTCSASSGVNFMHWYQQKSGTSPKRWIFDTSK |
| | chain | | LASGVPARFSGSGSGTSYSLTISSMEAEDAATYYCQQWSFNPPTFGGGTKLEIK |
| | | | R |
| BCMA | VH | 174 | EVQLLESGGGLVQPGGSLRLSCAASGFTFSSYAMNWVRQAPGKGLE |
| | | | WVSAILSSGGSTYYADSVKGRFTISRDNSKNTLYLQMNSLRAEDTAVYY |
| | | | CARYWPMDIWGQGTLVTV |
| | VL | 175 | EIVLTQSPGTLSLSPGERATLSCRGGQSVSSSYLAWYQQKPGQAPRLL |
| | | | MYDASIRATGIPDRFSGSGSGTDFTLTISRLEPEDFAVYYCQQYQSWPL |
| | | | TFGQGTKVEIK |

In another preferred embodiment, said VH and VL chains have as epitope target sequence of over 80% identity, preferably over 90%, and more preferably over 95% with SEQ ID NO 11 (CD20 antigen).

The extracellular ligand-binding domain can also comprise a peptide binding an antigen of the target, a peptide or a protein binding an antibody that binds an antigen of the target, a peptide or a protein ligand such as a growth factor, a cytokine or a hormone as non-limiting examples binding a receptor on the target, or a domain derived from a receptor such as a growth factor receptor, a cytokine receptor or a hormone receptor as non-limiting examples, binding a peptide or a protein ligand on the target. Preferably the target is a cell or a virus.

The antigen binding domain of the CAR can be any domain that binds to the cell target antigen including but not limited to a monoclonal antibody, a recombinant antibody, a human antibody, a humanized antibody, and a functional fragment thereof.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169: 1119-25 (2002), Caldas et al., Protein Eng., 13(5):353-60 (2000), Morea et al., Methods, 20(3): 267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8): 1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.).

According to the invention, the scFv may be nanobodies (natural single domain antibodies) which can be obtained by immunization of dromedaries, camels, llamas, alpacas or sharks.

Linkers within the Chimeric scFv

The flexibility of scFv linker engineering can be combined with the inherent quick and adaptable characters of surface coupling chemistry (e.g., electrostatic, hydrogen bonding, or covalent attachment). Peptide linkers can vary from 10 to 25 amino acids in length and are typically, but not always, composed of hydrophilic amino acids such as glycine (G) and serine (S). Peptide linkers of shorter lengths (0-4 amino acids) have also been used. However, scFv bearing shorter linkers can form multimers. Generally, the (GGGGS)₃ peptide is used as an scFv peptide linker. This 15-amino acid linker sequence [designated as the (GGGGS) 3 linker] is used in the Recombinant Phage Antibody System (RPAS kit) commercially available from Amersham. Previous study demonstrated that scFvs (MW˜27 000) containing metal-binding amino acids (i.e., cysteine or histidine) in the scFv peptide linker can be directly immobilized onto a gold surface in a favorable antigen-binding orientation at high density that significantly increased assay sensitivity by 3-5-fold over whole IgG or Fab antibody fragments, respectively (Shen Z, Mernaugh R L, Yan H, Yu L, Zhang Y, Zeng X. Anal. Chem. 2005; 77:6834-6842; Shen Z, Stryker G A, Mernaugh R L, Yu L, Yan H, Zeng X. Anal. Chem. 2005; 77:797-805).

Amongst other linkers suitable within the present invention are the 15-mer peptide linker (RGRGRGRGRSRGGGS) (Zhihong Shen, Heping Yan, Ying Zhang, Raymond L. Mernaugh, and Xiangqun Zeng (2008), Anal Chem. 80(6): 1910-1917).

In some embodiments, the “linker” as used in the context of a scFv refers to a peptide linker that consists of amino acids such as glycine and/or serine residues used alone or in combination, to link variable heavy and variable light chain regions together. In one embodiment, the flexible polypeptide linker is a Glycine/Serine linker and comprises the amino acid sequence (Gly-Gly-Gly-Ser)ₙ or (Gly-Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (GlyₓSer)ₙ, where x=1, 2, 3, 4 or 5 and n is 1, 2, 3, 4, 5, 6, 7, 8, 9 or 10, such as multiple repeat of (GlySer), (Gly₂Ser) or (GlysSer). Also included within the scope of the invention are linkers described in WO2012/138475, incorporated herein by reference.

### Chimeric Antigen Receptor (CAR)

The CAR according to the invention are sought for enabling engineered immune cells to trigger the destruction of pathological cells, in particular malignant cells. They may be designed according to single-chain or multi-chain architectures. In some embodiments, the extracellular ligand-binding domain, transmembrane domain, and intracellular signaling domain are in one polypeptide, i.e., in a single chain. Multi-chain architectures are more particularly disclosed in WO2014039523.

A multi-chain CAR is typically formed of different polypeptides such as:
one transmembrane polypeptide comprising at least one extracellular ligand-binding domain and;
one transmembrane polypeptide comprising at least one signal-transducing domain.

The signaling polypeptide is responsible for the activation of at least one of the normal functions of the engineered immune cell. For example, the function of a T cell can be a cytolytic activity or helper activity including the secretion of cytokines. Thus, the term “signaling protein” refers to a protein which transduces the transmitter domain function signal and directs the cell to perform a specialized function. In a particular embodiment, said transmitter domain can be a signaling protein. Transmission of the signals can result from: protein/protein interactions, protein/DNA interaction, protein/RNA interaction, protein/small molecule interaction, post translational protein modification, conformational change, subcellular relocalization.

The signaling protein can activate a gene in the nucleus. Examples of signaling protein can be members of NFAT transcription factor family which are inducible factor that could bind the intereukin-2 promoter in activated T cells. The regulation of NFAT proteins involves metabolites and proteins such as calcium, calcineurin and Homer scaffolding proteins. Said signaling protein can be an activated engineered form of NFAT avoiding regulation by calcineurin and Homer proteins. Said signaling protein can be a NF-κB engineered to avoid sequestration in the cytoplasm by Iκb allowing activation of T cells. Said signaling protein can also be the expression of the three IKK subunits (IKKα, IKKβ, IKKγ). Reconstituted IKK complex activated NF-κB pathway, by triggering the ubiquitination of the IκB. Also the activation of the JNK signaling could be triggered through the direct expression of signaling protein AP-1 (transcription factor). Said signaling protein can be an engineered transcription activator like effector (TALE) binding domain that will specifically target and activate transcription of the same gene as for the NFAT and NF-kb.

According to the invention, said signaling protein can inhibit a signaling pathway through protein-protein interaction or can activate a gene in the nucleus to inhibit a signaling pathway. Said signaling protein can be vaccinia H1 related proteins (VHR) a member of the mitogen-activated protein kinase phosphatases (MKPs) family which dephosphorylates and inactivates an extracellular signal regulated kinases (ERK) signaling proteins.

According to the invention, signal transducing domain for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain may comprise two distinct classes of cytoplasmic signaling sequence, those that initiate antigen-dependent primary activation, and those that act in an antigen-independent manner to provide a secondary or co-stimulatory signal.

In particular embodiment the signal transduction domain of the CAR of the present invention comprises a co-stimulatory signal molecule. A co-stimulatory molecule is a cell surface molecule other than an antigen receptor or their ligands that is required for an efficient immune response.

“Co-stimulatory ligand” refers to a molecule on an antigen presenting cell that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A “co-stimulatory molecule” refers to the cognate binding partner on a T cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor.

For instance, a multi-chain CAR can be derived from the structure of a Fc receptor, preferably FcεRI, and comprise at least two of the following components:
a) one polypeptide comprising the transmembrembrane domain of FcRI alpha chain fused to an extracellular ligand-binding domain,
b) one polypeptide comprising a part of N- and C-terminal cytoplasmic tail fused to the transmembrane domain of a FcRI beta chain, and/or
c) two additional polypeptides comprising each one part of an intracytoplasmic tail and/or the transmembrane domain of FRI gamma chain,

In general, these different polypeptides multimerize together spontaneously to form dimeric, trimeric or tetrameric structures that arise at the cell surface in a juxtamembrane position.

In some embodiments, the invention relates to an immune cell comprising a single-chain CAR as well defined in the prior art, as well as in any of U.S. Pat. No. 7,446,190, WO2008/121420, U.S. Pat. No. 8,252,592, US20140024809, WO2012/079000, WO2014153270, WO2012/099973, WO2014/011988, WO2014/011987, WO2013/067492, WO2013/070468, WO2013/040557, WO2013/126712, WO2013/126729, WO 2013/126726, WO2013/126733, U.S. Pat. No. 8,399,645, US20130266551, US20140023674, WO2014039523, U.S. Pat. Nos. 7,514,537, 8,324,353, WO2010/025177, U.S. Pat. No. 7,446,179, WO2010/025177, WO2012/031744, WO2012/136231A1, WO2012/050374A2, WO2013074916, WO/2009/091826A3, WO2013/176915 or WO/2013/059593.

In some embodiments, the invention relates to a CAR comprising
an extracellular domain comprising
   at least one extracellular binding domain that comprises a scFv formed by at least a VH chain and a VL chain specific to an antigen, preferably a cell surface marker antigen, wherein said extracellular binding domain comprises at least one mAb-specific epitope, and,
   a hinge,
a transmembrane domain, and,
an intracellular domain.

In one embodiment, the transmembrane domain comprises the transmembrane region(s) of the alpha, beta or zeta chain of the T-cell receptor, PD-1, 4-1BB, OX40, ICOS, CTLA-4, LAG3, 2B4, BTLA4, TIM-3, TIGIT, SIRPA, CD28, CD3 epsilon, CD45, CD4, CD5, CD8, CD9, CD16, CD22, CD33, CD37, CD64, CD80, CD86, CD134, CD137 or CD154.

In another embodiment, the hinge is an IgG4 hinge or a CD8 alpha hinge, preferably a CD8 alpha hinge.

The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T cell receptor such as α, β, γ or δ, polypeptide constituting CD3 complex, IL2 receptor p55 (α chain), p75 (β chain) or γ chain, subunit chain of Fc receptors, in particular Fcγ receptor III or CD proteins. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1). Said transmembrane domain can also be a CD8 transmembrane domain (alpha and beta chains). Said Transmembrane domain can be engineered to create obligated hetero or homodimers. In particular embodiment said CARs can comprise transmembrane domains or intracellular domains which can only dimerize after ligand recognition. Another example of transmembrane domain can be NKG2-D receptor. NKG2D (natural killer cell group 2D) is a C-type lectin-like receptor expressed on NK cells, γδ-TcR⁺ T cells, and CD8⁺αβ-TcR⁺ T cells (Bauer, Groh et al., 1999, Science 285(5428):727-9. NKG2D is associated with the transmembrane adapter protein DAP10 (Wu, Song et al. 1999, Science 285(5428):730-2), whose cytoplasmic domain binds to the p 85 subunit of the PI-3 kinase.

Said transmembrane domain can also be an integrin. Integrins are heterodimeric integral membrane proteins composed of a α and β chains which combined together form the LFA-1 (integrin lymphocyte function-associated antigen-1) which is expressed on all leukocytes. LFA-1 plays a central role in leukocyte intercellular adhesion through interactions with its ligand, ICAMs 1-3 (intercellular adhesion molecules 1 through 3), and also it has an important role in lymphocyte co-stimulatory signaling (Chen and Flies 2013, Nat Rev Immunol 13(4):227-42). The molecular details of the binding of LAF-1 to its immunoglobulin ICAM-1 are quite known allowing a careful engineering of LAF-1 binding site. The affinity of ox domain for ICAM-1 is regulated by the displacement of its C-terminal helix which is conformational linked to alterations of specific loops in LAF-1. The active and low conformations differ of 500 and 10,000 folds. It is also interesting to note that two types of antagonists are known for LFA-1 and their mechanism of action is known. Integrin cell surface adhesion receptors can transmit a signal from the outside to inside but also vice-versa. There are cytoskeletal proteins as Talin which binds to the integrin tail LFA-1 to transfer a message from inside to outside.

According to one embodiment, the transmembrane domain comprises the transmembrane region of PD-1 or the transmembrane region(s) of CD8 alpha.

In one aspect of the invention, the transmembrane domain is attached to the extracellular domain of the CAR via a hinge e.g., a hinge from a human protein. For example, in one embodiment, the hinge can be a human Ig (immunoglobulin) hinge, e.g., a PD-1 hinge, an IgG4 hinge, or a CD8alpha hinge.

In a preferred embodiment, the hinge of the CAR is a human immunoglobulin hinge.

In a more preferred embodiment, the hinge of the CAR is an IgG4 hinge or a CD8 alpha hinge.

In some embodiments, the hinge is an FcγRIII alpha hinge.

In some embodiments, the hinge is a CD8 alpha hinge.

In some embodiments, the hinge is a CD8 alpha hinge has amino acid sequence with at least about 70%, preferably at least 80%, more preferably at least 90%, 95%, 97%, or 99% sequence identity with an amino acid sequence shown in SEQ. ID NO: 179, 180 or 181.

The term “hinge region” (also named stalk region in the literature) used herein generally means any oligo- or polypeptide that functions to link the transmembrane domain to the extracellular ligand-binding domain. In particular, stalk region are used to provide more flexibility and accessibility for the extracellular ligand-binding domain. A stalk region may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4, CD28 or RTK, or from all or part of an antibody constant region. Alternatively the stalk region may be a synthetic sequence that corresponds to a naturally occurring stalk sequence, or may be an entirely synthetic stalk sequence.

The intracellular domain (also referred to herein as a “cytoplasmic signaling domain” or “an intracellular signaling domain”) comprises a functional signaling domain derived from a stimulatory molecule as defined below. In some embodiments, the stimulatory molecule is the zeta chain associated with the T-cell receptor complex. In some embodiments, the cytoplasmic signaling domain further comprises one or more functional signaling domains derived from at least one costimulatory molecule as defined below. In some embodiments, the costimulatory molecule is chosen from 4-1BB (i.e., CD137), CD27 and/or CD28.

The term “stimulatory molecule,” refers to a molecule expressed by a T-cell that provides the positive cytoplasmic signaling sequence(s) that regulate positive activation of the TCR complex in a stimulatory way for at least some aspect of the T-cell signaling pathway. In some embodiments, the positive signal is initiated by, for instance, binding of a TCR/CD3 complex with an MHC molecule loaded with peptide, and which leads to mediation of a T-cell response, including, but not limited to, proliferation, activation, differentiation, and the like. A positive cytoplasmic signaling sequence (also referred to as a “positive signaling domain” or positive intracellular signaling domain) that acts in a stimulatory manner may contain a signaling motif which is known as immunoreceptor tyrosine-based activation motif or ITAM. Examples of an ITAM containing positive cytoplasmic signaling sequence includes, but is not limited to, those derived from TCR zeta (or CD3zeta), FcR gamma, FcR beta, CD3 gamma, CD3 delta, CD3 epsilon, CD5, CD22, CD79a, CD79b, CD278 (also known as “ICOS”) and CD66d. In some embodiments, the intracellular signaling domain of the CAR can comprise the CD3ζ (zeta) signaling domain which has amino acid sequence with at least about 70%, preferably at least 80%, more preferably at least 90%, 95%, 97%, or 99% sequence identity with an amino acid sequence shown in SEQ. ID NO: 175.

In some aspect, the intracellular signaling domain of the CAR generates a signal that promotes an immune effector function of the CAR containing cell. Examples of immune effector function, e.g., in a CAR T-cell, include cytolytic activity and helper activity, including the secretion of cytokines.

The term “costimulatory molecule” refers to the cognate binding partner on a T-cell that specifically binds with a costimulatory ligand, thereby mediating a costimulatory response by the T-cell, such as, but not limited to, proliferation. Costimulatory molecules are cell surface molecules other than antigen receptors or their ligands that are required for an efficient immune response. Costimulatory molecules include, but are not limited to an MHC class I molecule, BTLA and a Toll ligand receptor, as well as OX40, CD2, CD27, CD28, CDS, ICAM-1, LFA-1 (CD11a/CD18) and 4-IBB (CD137).

A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. In some embodiments, the intracellular signaling domain of the CAR of the invention comprises amino acid sequence which comprises at least 70%, preferably at least 80%, more preferably at least 90%, 95%, 97%, or 99% sequence identity with an amino acid sequence shown in SEQ. ID NO: 176 and SEQ. ID NO: 177.

TABLE 4
provide exemplary sequence of CAR components

| | | SEQ ID |
|---|---|---|
| Domain | Amino Acid Sequence | NO: |
| CD8α signal peptide | MALPVTALLLPLALLLHAARP | 178 |
| FcγRIIIα hinge | GLAVSTISSFFPPGYQ | 179 |
| CD8α hinge | TTTPAPRPPTPAPTIASQPLSLRPEACRPAAGGAVHTRGLDFACD | 180 |
| IgG1 hinge | EPKSPDKTHTCPPCPAPPVAGPSVFLFPPKPKDTLMIARTPEVTCVVVD | 181 |
| | VSHEDPEVKFNWYVDGVEVHNAKTKPREEQYNSTYRVVSVLTVLHQD | |
| | WLNGKEYKCKVSNKALPAPIEKTISKAKGQPREPQVYTLPPSRDELTKN | |
| | QVSLTCLVKGFYPSDIAVEWESNGQPENNYKTTPPVLDSDGSFFLYSKLT | |
| | VDKSRWQQGNVFSCSVMHEALHNHYTQKSLSLSPGK | |
| CD8α | IYIWAPLAGTCGVLLLSLVITLYC | 182 |
| transmembrane | | |
| (TM) domain | | |
| 41BB intracellular | KRGRKKLLYIFKQPFMRPVQTTQEEDGCSCRFPEEEEGGCEL | 176 |
| signaling domain | | |
| (ISD) | | |
| CD3ζ intracellular | RVKFSRSADAPAYQQGQNQLYNELNLGRREEYDVLDKRRGRDPEMGG | 175 |
| signaling domain | KPRRKNPQEGLYNELQKDKMAEAYSEIGMKGERRRGKGHDGLYQGLS | |
| (ISD) | TATKDTYDALHMQALPPR | |
| FcϵR1 α-TM-IC | FFIPLLVVILFAVDTGLFISTQQQVTFLLKIKRTRKGFRLLNPHPKPNPKNN | 183 |
| (FcϵR1 α chain | | |
| transmembrane and | | |
| intracellular | | |
| domain) | | |
| FcϵRI β-ΔITAM | MDTESNRRANLALPQEPSSVPAFEVLEISPQEVSSGRLLKSASSPPLHTW | 184 |
| (FcϵRIβ chain | LTVLKKEQEFLGVTQILTAMICLCFGTVVCSVLDISHIEGDIFSSFKAGYPF | |
| without ITAM) | WGAIFFSISGMLSIISERRNATYLVRGSLGANTASSIAGGTGITILIINLKKS | |
| | LAYIHIHSCQKFFETKCFMASFSTEIVVMMLFLTILGLGSAVSLTICGAGE | |
| | ELKGNKVPE | |
| 41BB-IC (41BB co- | KRGRKKLLYIFKQPFMRPVQTTQEEDGCSCRFPEEEEGGCEL | 177 |
| stimulatory domain) | | |
| CD28-IC (CD28 co- | RSKRSRGGHSDYMNMTPRRPGPTRKHYQPYAPPRDFAAYRS | 185 |
| stimulatory domain) | | |

CARs and immune cells comprising them have been extensively disclosed and can be prepared by the skilled person according to known methods. For example, methodologies to prepare CARs and cells comprising such CARs are disclosed in U.S. Pat. No. 7,446,190, WO2008/121420, U.S. Pat. No. 8,252,592, US20140024809, WO2012/079000, WO2014153270, WO2012/099973, WO2014/011988, WO2014/011987, WO2013/067492, WO2013/070468, WO2013/040557, WO2013/126712, WO2013/126729, WO 2013/126726, WO2013/126733, U.S. Pat. No. 8,399,645, US20130266551, US20140023674, WO2014039523, U.S. Pat. Nos. 7,514,537, 8,324,353, WO2010/025177, U.S. Pat. No. 7,446,179, WO2010/025177, WO2012/031744, WO2012/136231A1, WO2012/050374A2, WO2013074916, WO2009/091826A3, WO2013/176915 or WO/2013/059593 which are all incorporated herein in their entirety by reference.

The present invention encompasses a recombinant DNA construct comprising sequences encoding an CAR as defined above, wherein the CAR comprises an extracellular domain such as an antibody fragment that binds specifically to cell target antigen, and wherein the sequence of the extracellular domain is contiguous with and in the same reading frame as a nucleic acid sequence encoding a transmembrane domain and an intracellular domain. An exemplary CAR construct may comprise an optional leader sequence, an extracellular cell target antigen binding domain, a hinge, a transmembrane domain, and an intracellular inhibitory signaling domain

In some embodiments, the invention relates to a recombinant DNA construct comprising sequences encoding a CAR as defined above. In some embodiments, the CAR comprises an extracellular domain comprising
at least one extracellular binding domain that comprises a scFv formed by at least a VH chain and a VL chain specific to a cell surface marker antigen, wherein said extracellular binding domain comprises at least one mAb-specific epitope to be bound by a epitope-specific mAb for in vitro cell sorting and/or in vivo cell depletion of T cells expressing said CAR and wherein comprising comprising an extracellular binding domain, and,
a hinge,
a transmembrane domain, and,
an intracellular domain.

### Method for Sorting CAR-Positive Immune Cells

According to one aspect, the invention relates to a method for in vitro sorting CAR-expressing immune cell, comprising contacting a population of said engineered immune with antigen-specific antibody (preferably monoclonal Abs) to collect only cells expressing CAR.

In some embodiments, the invention relates to a method for in vitro sorting CAR-expressing immune cell, wherein said CAR comprises at least one extracellular binding domain comprising at least one mAb-specific epitope as described above, comprising
contacting a population of said immune cells with a monoclonal antibody specific for said mAb-specific epitope to collect only said CAR-expressing immune cell.

In some embodiments, the invention relates to a method for in vitro sorting CAR-expressing immune cells, wherein said CAR comprises at least one extracellular binding domain comprising at least one mAb-specific epitope, comprising
contacting a population of said immune cells with a monoclonal antibody (epitope-specific mAb) specific for said mAb-specific epitope,
selecting the cells that bind to the monoclonal antibody, to obtain a population of cells enriched in CAR-expressing immune cell.

In some embodiments, said monoclonal antibody specific for said mAb-specific epitope is conjugated to a fluorophore and the step of selecting the cells that bind to the monoclonal antibody is done by Fluorescence Activated Cell Sorting (FACS).

In some embodiments, said monoclonal antibody specific for said mAb-specific epitope is conjugated to a magnetic particle and the step of selecting the cells that bind to the monoclonal antibody is done by Magnetic Activated Cell Sorting (MACS).

In some embodiments, the extracellular binding domain of the CAR comprises a mAb-specific epitope of SEQ ID NO 144.

In some embodiments, the extracellular binding domain of the CAR comprises a mAb-specific epitope of SEQ ID NO 144 and the antibody used to contact the population of immune cells is QBEND-10.

In some embodiments, the extracellular binding domain of the CAR comprises a mAb-specific epitope of SEQ ID NO 35.

In some embodiments, the extracellular binding domain of the CAR comprises a mAb-specific epitope of SEQ ID NO 35 and the antibody used to contact the population of immune cells is

### Rituximab.

In some embodiments, the population CAR-expressing immune cells obtained when using the method for in vitro sorting CAR-expressing immune cells described above, comprises at least 70%, 75%, 80%, 85%, 90%, 95% of CAR-expressing immune cells. In some embodiments, the population

CAR-expressing immune cells obtained when using the method for in vitro sorting CAR-expressing immune cells described above, comprises at least 85% CAR-expressing immune cells.

In some embodiments, the population of CAR-expressing immune cells obtained when using the method for in vitro sorting CAR-expressing immune cells described above shows increased cytotoxic activity in vitro compared with the initial (non-sorted) cell population using the protocol described in Example 7.5. In a preferred embodiment, said cytotoxic activity in vitro is increased by 10%, 20%, 30% or 50%.

Preferably, the mAbs are previously bound onto a support such as a column or on beads such as routinely realized by the skilled in the art.

According to a favored embodiment, immune cells are T-cells.

According to the invention, cells to be administered to the recipient may be enriched in vitro from the source population.

Methods of expanding source populations are well known in the art, and may include selecting cells that express an antigen such as CD34 antigen, using combinations of density centrifugation, immuno-magnetic bead purification, affinity chromatography, and fluorescent activated cell sorting, known to those skilled in the art.

### Flow Cytometry

Flow cytometry is widely used in the art and is a method well known to one of ordinary skill to sort and quantify specific cell types within a population of cells. In general, flow cytometry is a method for quantitating components or structural features of cells primarily by optical means. Since different cell types can be distinguished by quantitating structural features, flow cytometry and cell sorting can be used to count and sort cells of different phenotypes in a mixture.

A flow cytometric analysis involves two basic steps: 1) labeling selected cell types with one or more labeled markers, and T) determining the number of labeled cells relative to the total number of cells in the population.

The primary method of labeling cell types is by binding labeled antibodies to markers expressed by the specific cell type. The antibodies are either directly labeled with a fluorescent compound or indirectly labeled using, for example, a fluorescent-labeled second antibody which recognizes the first antibody.

In a preferred embodiment, the method used for sorting T cells expressing CAR is the Magnetic-Activated Cell Sorting (MACS).

Magnetic-activated cell sorting (MACS) is a method for separation of various cell populations depending on their surface antigens (CD molecules) by using superparamagnetic nanoparticles and columns. It takes only a few simple steps to get pure cell populations Cells in a single-cell suspension are magnetically labeled with microbeads. The sample is applied to a column composed of ferromagnetic spheres, which are covered with a cell-friendly coating allowing fast and gentle separation of cells. The unlabeled cells pass through while the magnetically labeled cells are retained within the column. The flow-through can be collected as the unlabeled cell fraction. After a short washing step, the column is removed from the separator, and the magnetically labeled cells are eluted from the column.

Amongst other technique, FACS is a technique of choice to purify cell populations of known phenotype as very high purity of the desired population can be achieved, or when the target cell population expresses a very low level of the identifying marker, or when cell populations require separation based on differential marker density. In addition, FACS is the only available purification technique to isolate cells based on internal staining or intracellular protein expression, such as a genetically modified fluorescent protein marker. FACS allows the purification of individual cells based on size, granularity and fluorescence. In order to purify cells of interest, they are first stained with fluorescently-tagged monoclonal antibodies (mAb), which recognize specific surface markers on the desired cell population.

Detailed protocol for the purification of specific cell population such as T-cell can be found in Basu S et al. (2010). (Basu S, Campbell H M, Dittel B N, Ray A. Purification of specific cell population by fluorescence activated cell sorting (FACS). J Vis Exp. (41): 1546).

In a preferred embodiment of the invention, the mAb used in the method for sorting T cells expressing the CAR is chosen amongst ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.

In a more preferred embodiment, said mAb is rituximab.

In a more preferred embodiment, said mAb is QBEND-10.

### Method for Depleting CAR-Expressing Immune Cells

By “in vivo depletion” is meant in the present invention the administration of a treatment to a mammalian organism aiming to stop the proliferation of CAR-expressing immune cells by inhibition or elimination.

One aspect of the invention is related to a method for in vivo depleting an engineered immune cell expressing a CAR comprising an m-Ab specific epitope as previously described, comprising contacting said engineered immune cell or said CAR-expressing immune cell with at least one epitope-specific mAbs. Another aspect of the invention relates to a method for in vivo depleting immune CAR-expressing immune cell which comprises the above chimeric scFv (formed by insertion of a mAb-specific epitope) by contacting said engineered immune cell with epitope-specific antibodies.

Preferably, said immune cells are T-cells and/or the antibodies are monoclonal.

According to one embodiment, the in vivo depletion of immune engineered cell is performed on engineered immune cell which has been previously sorted using the in vitro method of the present invention. In this case, this will be the same infused mAb used.

According to a preferred embodiment, the mAb-specific antigen is CD20 antigen and the epitope-specific mAb is rituximab.

In some embodiments, the invention relates to a method for in vivo depleting an engineered immune cell expressing a CAR comprising an mAb-specific epitope (CAR-expressing immune cell) as previously described, in a patient comprising contacting said CAR-expressing immune cell with at least one epitope-specific mAb.

In a preferred embodiment of the invention, the mAb used in the method for depleting an engineered immune cell expressing a CAR is chosen amongst ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.

In some embodiments, said mAb-specific epitope is a CD20 epitope or mimotope, preferably SEQ ID NO 35 and the epitope-specific mAbs is rituximab.

In some embodiments, the step of contacting said engineered immune cell or said CAR-expressing immune cell with at least one epitope-specific mAb comprises infusing the patient with epitope-specific mAb, preferably rituximab. In some embodiments, the amount of epitope-specific mAb administered to the patient is sufficient to eliminate at least 20%, 30%, 40%, 50%, 60%, 70%, 80% or 90% of the CAR-expressing immune cell in the patient.

In some embodiments, the step of contacting said engineered immune cell or said CAR-expressing immune cell with at least one epitope-specific mAb comprises infusing the patient with 375 mg/m² of rituximab, once or several times, preferably once weekly.

In some embodiments, when immune cells expressing a CAR comprising an mAb-specific epitope (CAR-expressing immune cells) are depleted in a CDC assay using epitope-specific mAb, the amount of viable CAR-expressing immune cells decreases, preferably by at least 10%, 20%, 30%, 40%, 50%, 60%, 70%, 80% or 90%. Preferably the CDC assay is the assay disclosed in Example 3, Example 4 or Example 7.4. In some embodiments, said mAb-specific epitope is a CD20 epitope or mimotope, preferably SEQ ID NO 35 and the epitope-specific mAbs is rituximab.

To one particular embodiment, the in vivo depletion of CAR-engineered immune cells is performed by infusing bi-specific antibodies. By definition, a bispecific monoclonal antibody (BsAb) is an artificial protein that is composed of fragments of two different monoclonal antibodies and consequently binds to two different types of antigen. These BsAbs and their use in immunotherapy have been extensively reviewed in Müller D and Kontermann R. E. (2010) Bispecific Antibodies for Cancer Immunotherapy, BioDrugs 24 (2): 89-98.

By “effector cell”, this term includes immune cells such as lymphocytes, macrophages, dendritic cells, natural killer cells (NK Cell), cytotoxic T lymphocytes (CTL).

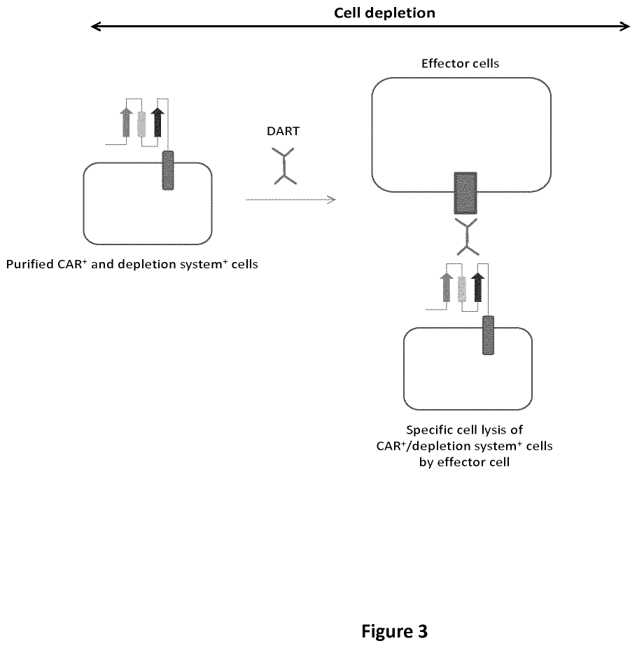

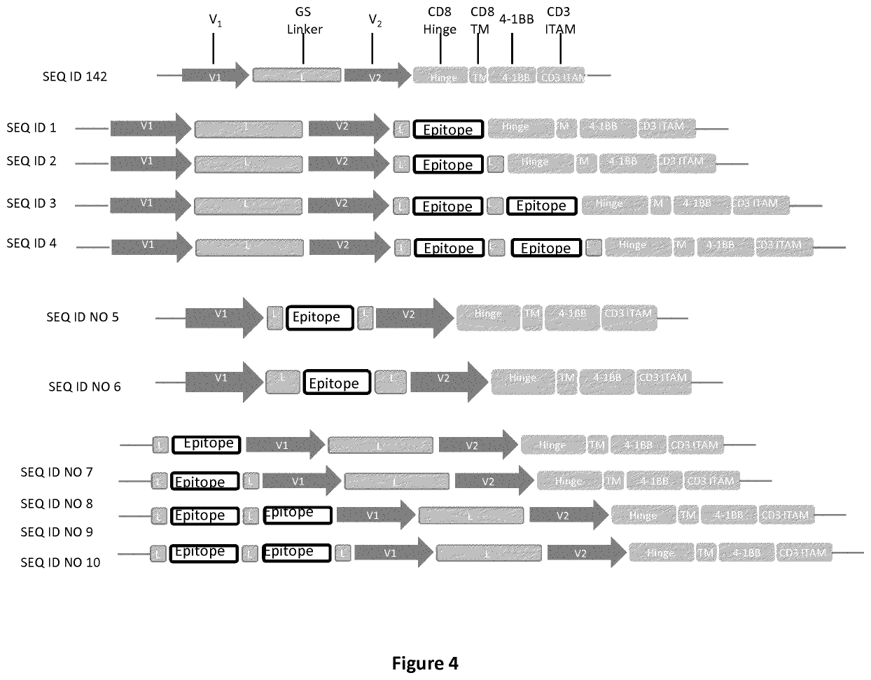

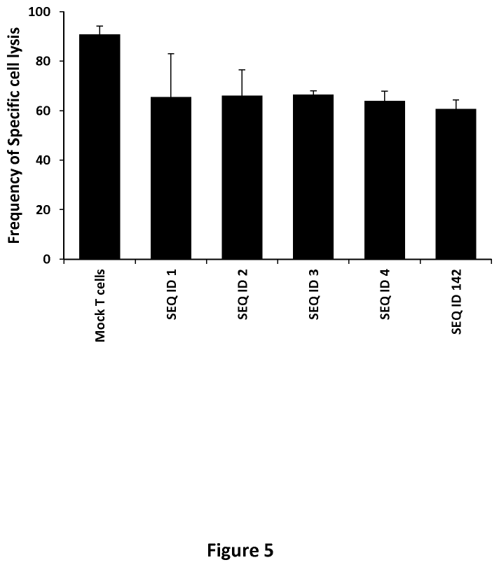

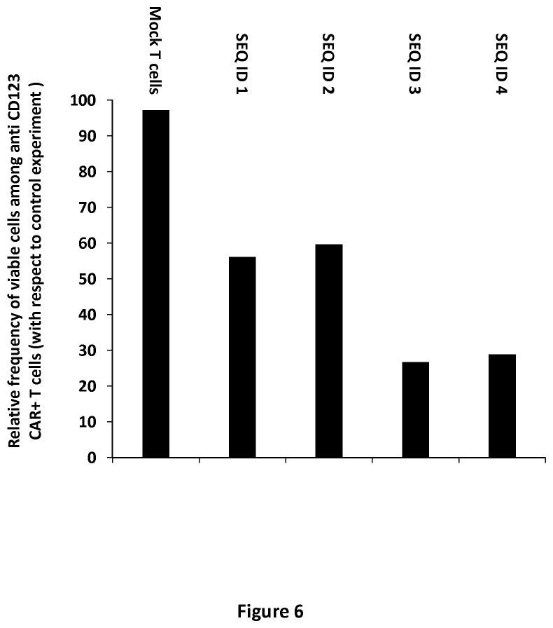

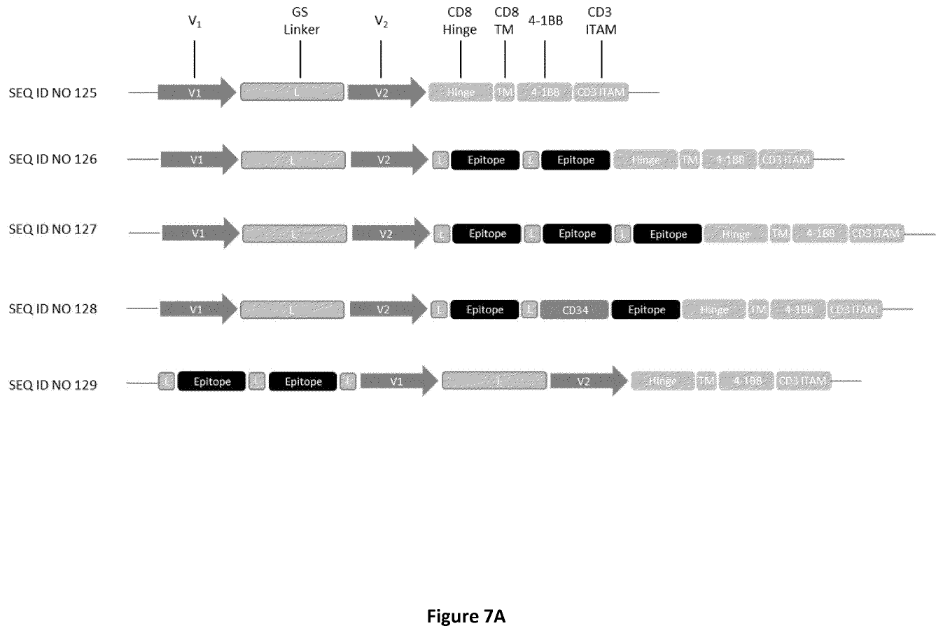

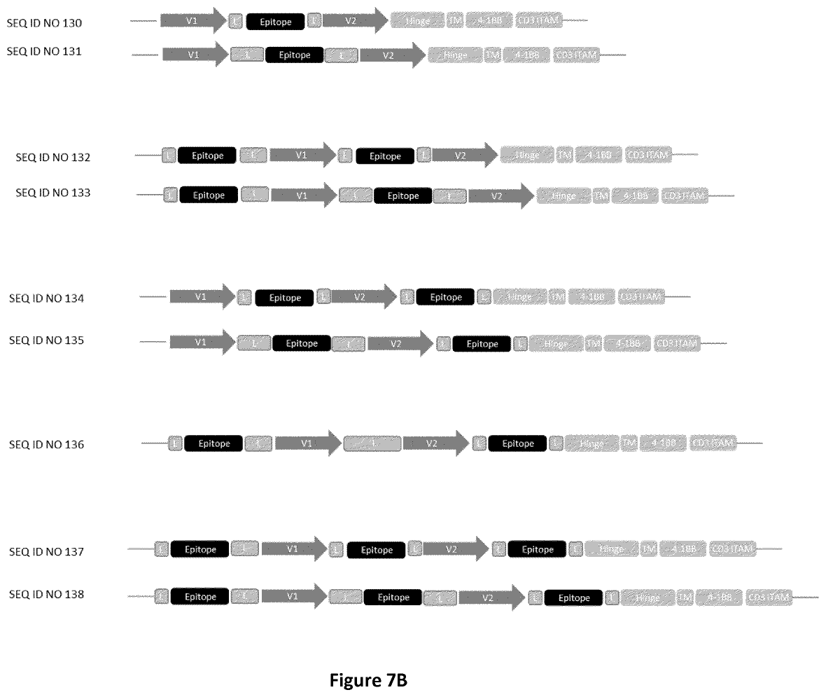

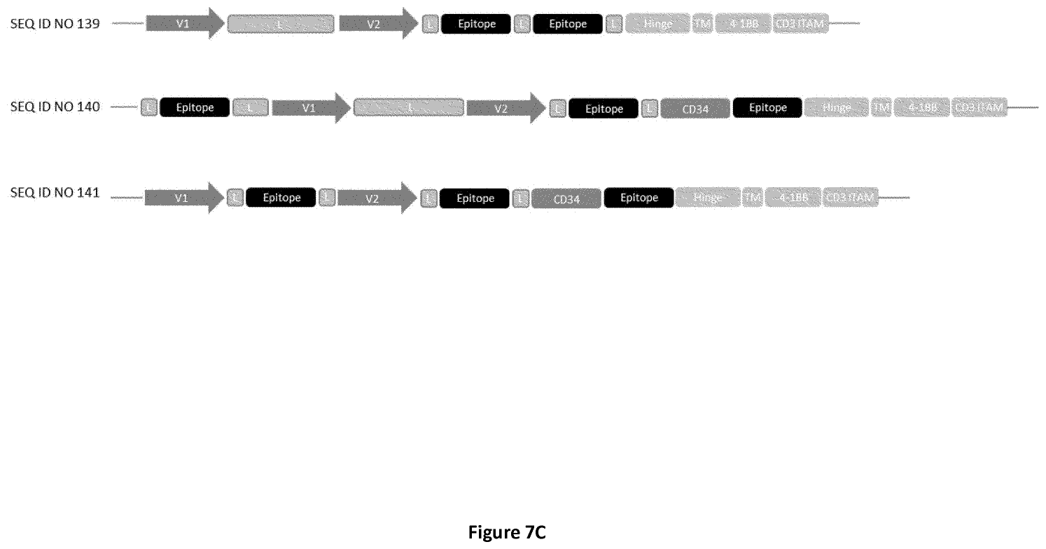

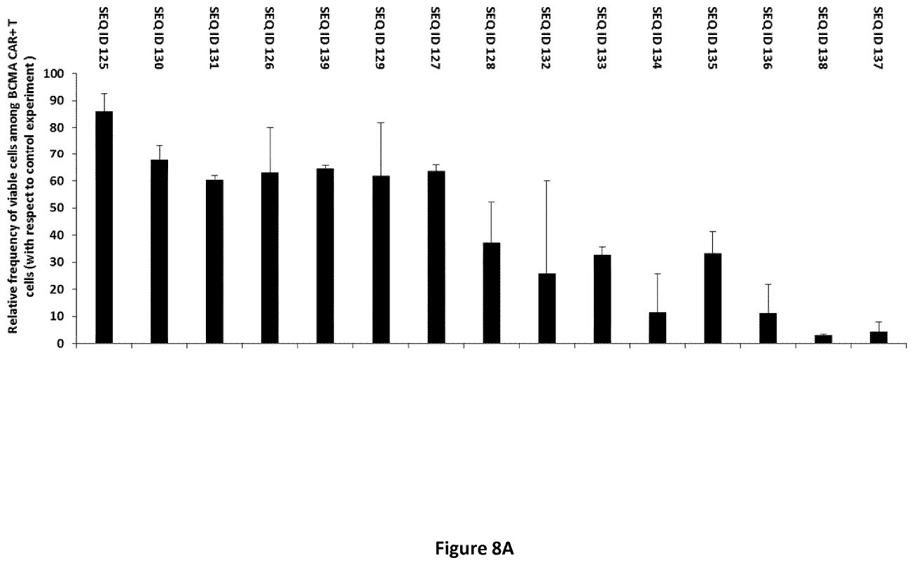

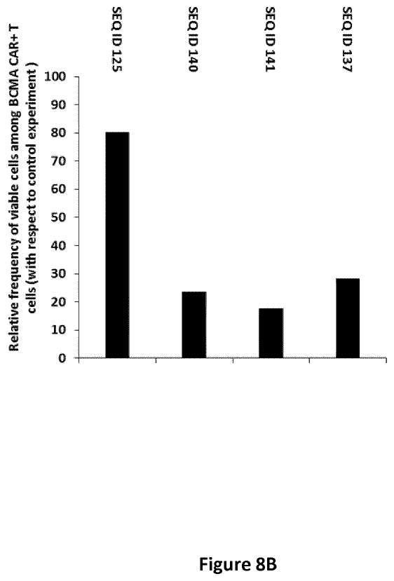

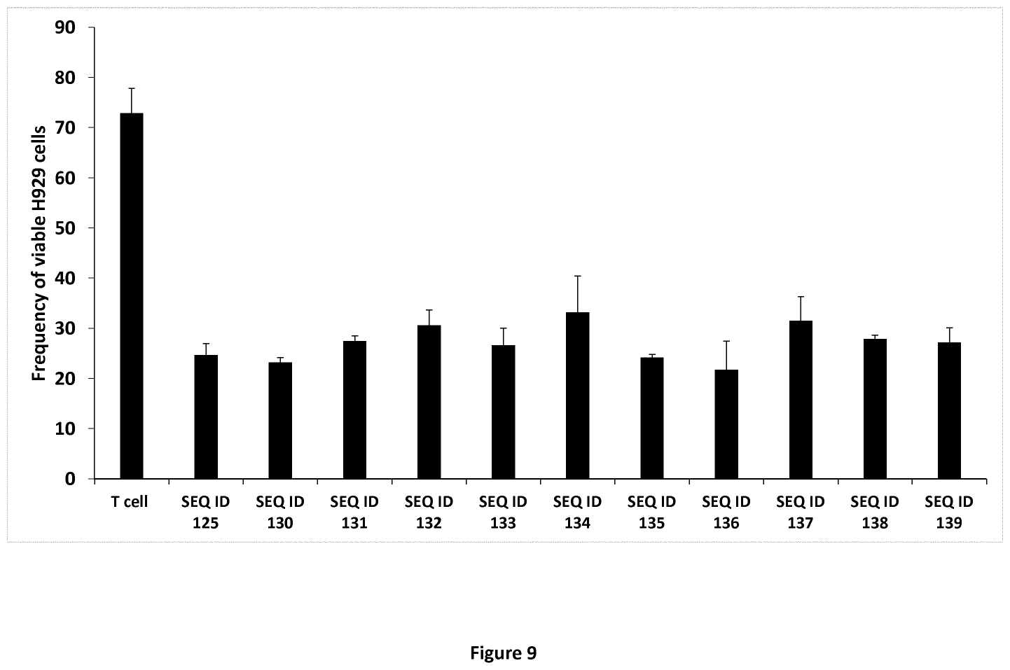

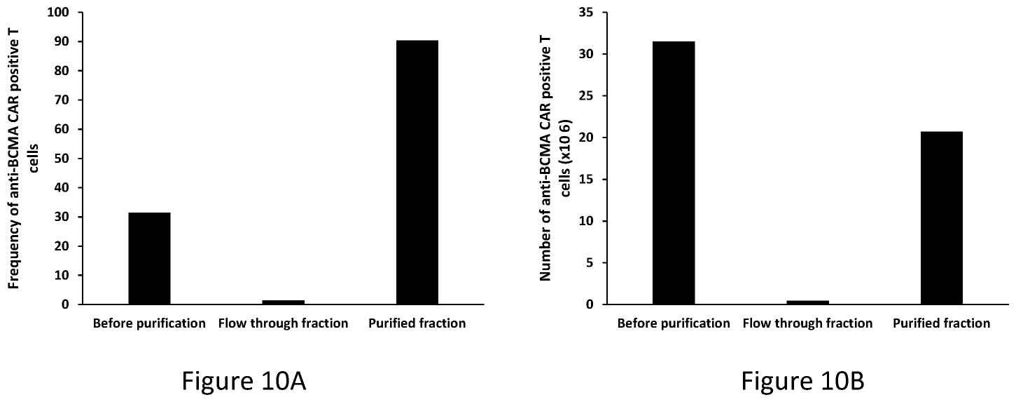

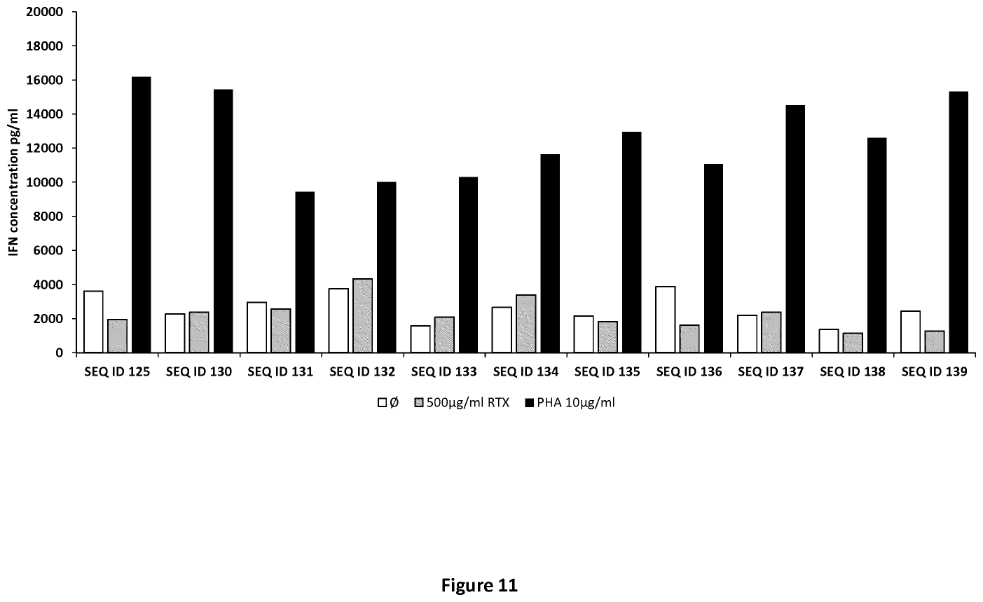

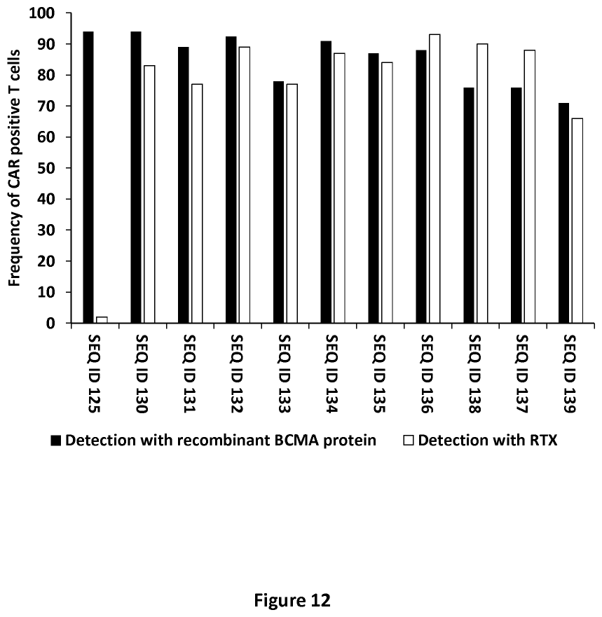

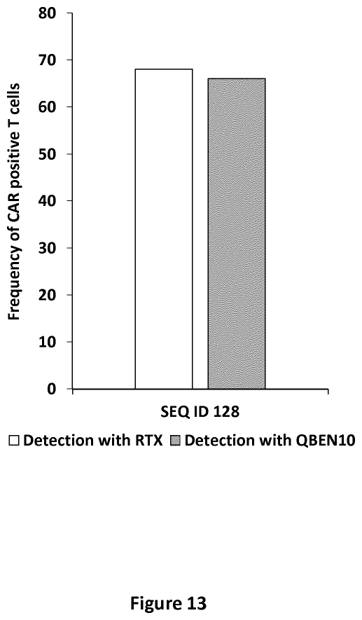

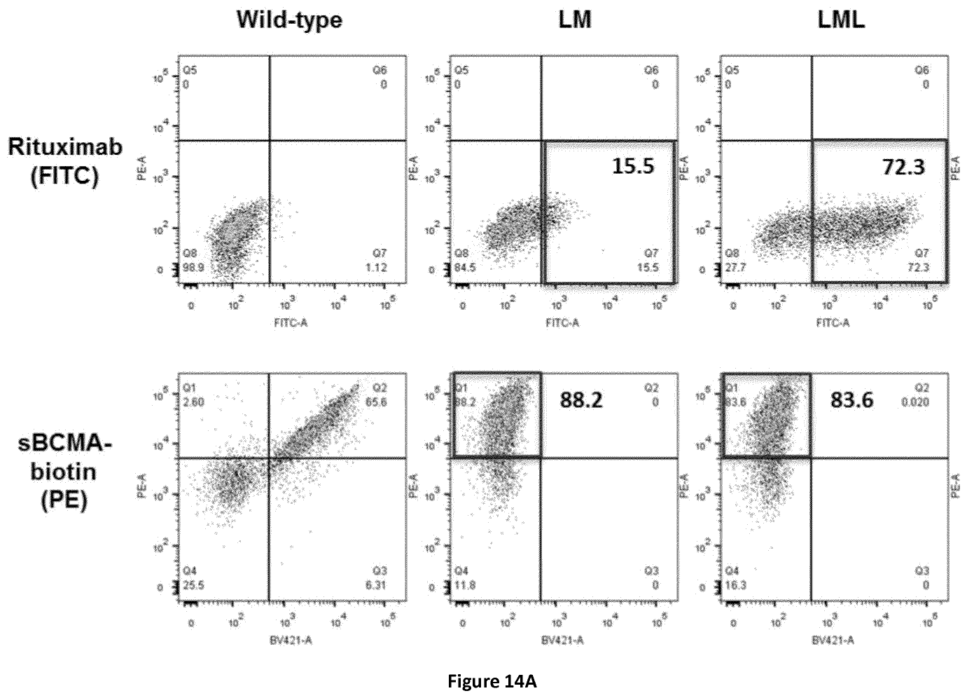

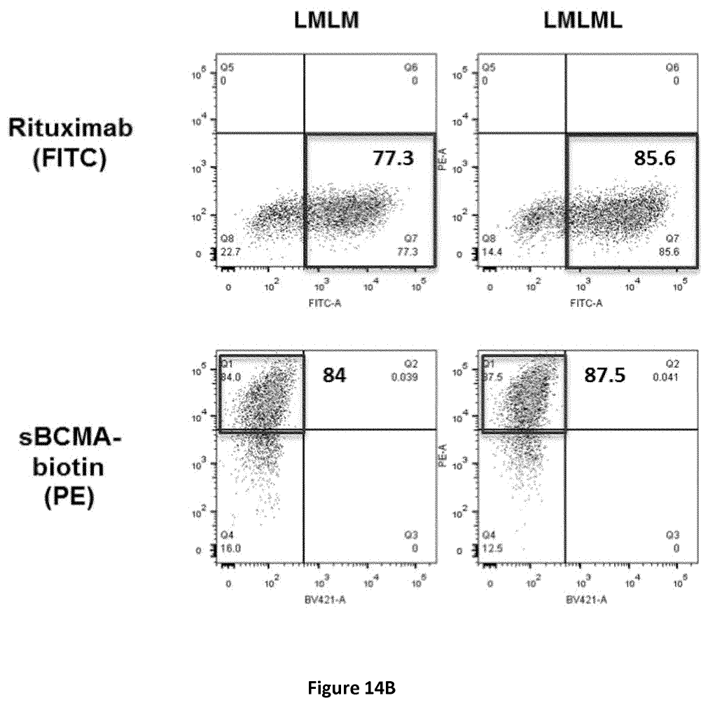

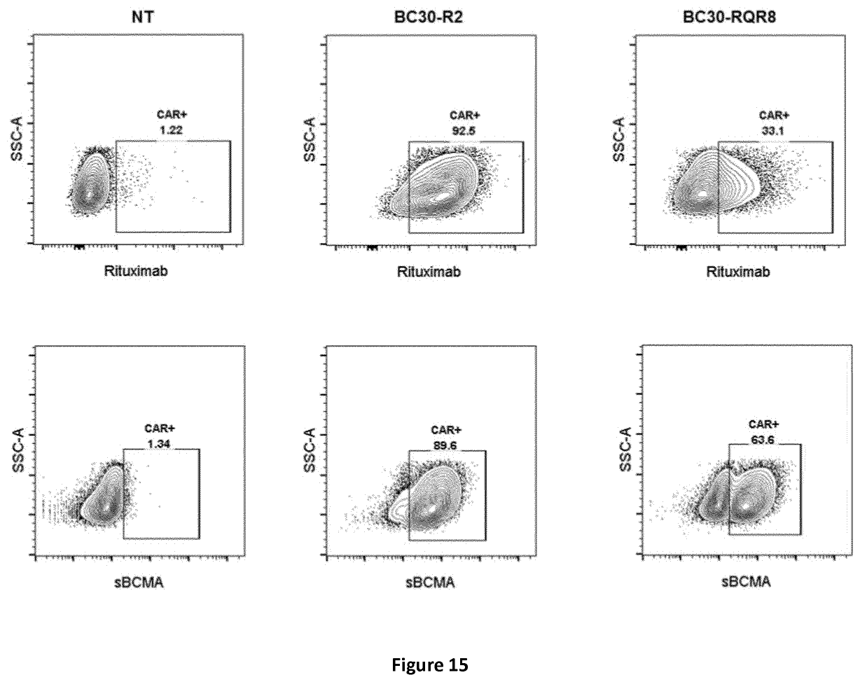

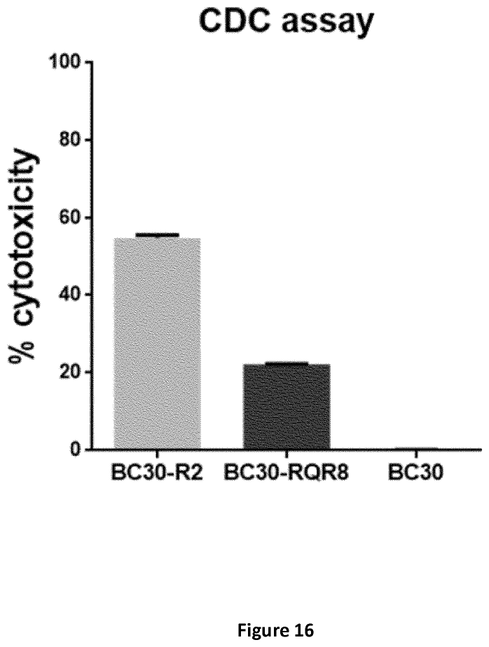

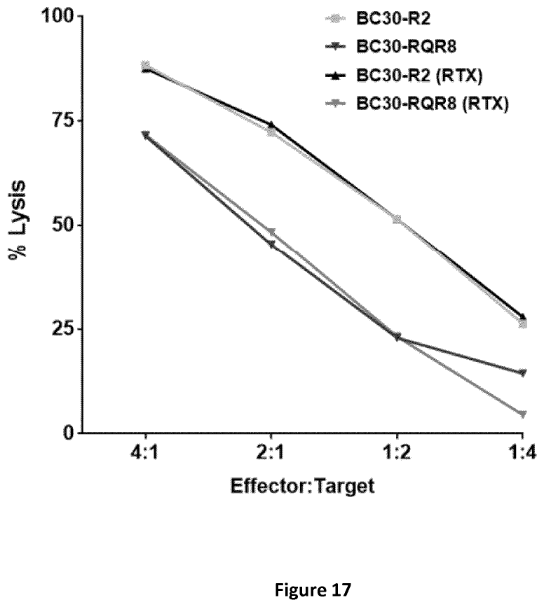

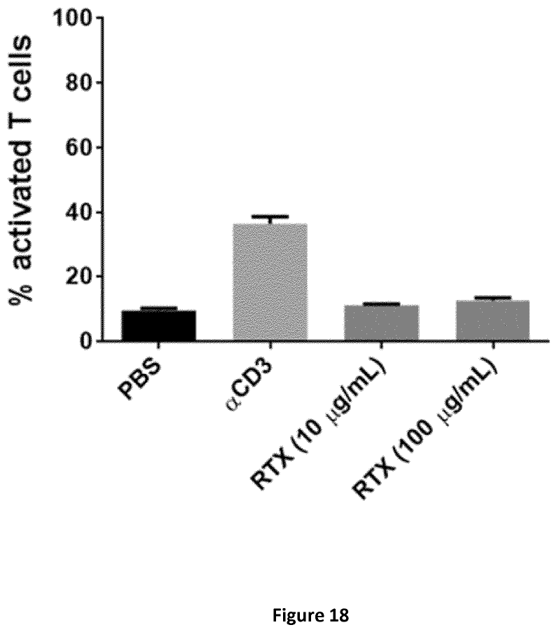

According to another particular embodiment, the infused bi-specific mAb is able to bind both the mAb-specific epitope borne on engineered immune cells expressing the chimeric scFv and to a surface antigen on an effector and cytotoxic cell. This aspect is presented in FIG. 3. By doing so, the depletion of engineered immune cells triggered by the BsAb can occur through antibody-dependent cellular cytotoxicity (ADCC). Such conformation can be found by instance in Deo Y M, Sundarapandiyan K, Keler T, Wallace P K, and Graziano R F, (2000), Journal of Immunology, 165 (10):5954-5961].

According to a particular embodiment, a cytotoxic drug is coupled to the epitope-specific mAbs which are used in to deplete CAR-expressing immune cells. By combining targeting capabilities of monoclonal antibodies with the cancer-killing ability of cytotoxic drugs, antibody-drug conjugate (ADC) allows a sensitive discrimination between healthy and diseased tissue when compared to the use of the drug alone. Market approvals were received for several ADCs; the technology for making them—particularly on linkers—is abundantly presented in the following prior art (Payne, G. (2003) Cancer Cell 3:207-212; Trail et al (2003) Cancer Immunol. Immunother. 52:328-337; Syrigos and Epenetos (1999) Anticancer Research 19:605-614; Niculescu-Duvaz and Springer (1997) Adv. Drug Del. Rev. 26:151-172; U.S. Pat. No. 4,975,278).

According to another particular embodiment, the epitope-specific mAb to be infused is conjugated beforehand with a molecule able to promote complement dependent cytotoxicity (CDC). Therefore, the complement system helps or complements the ability of antibodies to clear pathogens from the organism. When stimulated by one of several, is triggered an activation cascade as a massive amplification of the response and activation of the cell-killing membrane attack complex.

Different molecule may be used to conjugate the mAb, such as glycans [Courtois, A, Gac-Breton, S., Berthou, C., Guézennec, J., Bordron, A. and Boisset, C. (2012), Complement dependent cytotoxicity activity of therapeutic antibody fragments is acquired by immunogenic glycan coupling, Electronic Journal of Biotechnology ISSN: 0717-3458; http://www.ejbiotechnology.info DOI: 10.2225/vol15-issue5).

In some embodiments of the invention, the epitope-specific mAb used in the method for sorting and depleting an engineered immune cell expressing a CAR is the same and is chosen amongst ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and ustekinumab.

In some embodiments of the invention, different antibodies are used for sorting and depleting the cells. In some embodiments, the extracellular binding domain comprises at least one epitope specifically bound by rituximab such as an mAb-specific epitope having an amino acid sequence of SEQ ID NO 35 and at least one epitope specifically bound by QBEND10 such as SEQ ID NO 144 and the mAb used for sorting the cells is QBEND10 and the mAb used to deplete the cell is rituximab.

### Methods of Engineering Immune Cells

The inventors developed methods of engineering immune cells expressing a chimeric antigen receptor (CAR), preferably a CAR as described above, with all components necessary to trigger a cell surface target antigen and to expand/amplify. Further, this CAR has the particularity of to carry a chimeric scFv wherein the scFv is modified to include an epitope able to be specifically recognized by an antibody for cell sorting and/or cell depletion purposes.

In one embodiment, the method for engineering an immune cell chimeric antigen receptor (CAR), comprising at least one extracellular binding domain that comprises a scFv formed by at least a VH chain and a VL chain specific to a cell surface marker antigen and one mAb-specific epitope to be bound by a epitope-specific mAb, comprising:
(a) Providing an immune cell;
(b) Introducing into said cell at least one polynucleotide encoding the said chimeric antigen receptor.
(c) Expressing said polynucleotide into said cell.

In one embodiment, the method for engineering an immune cell expressing a chimeric antigen receptor (CAR) as described above, preferably comprising at least one extracellular binding domain that comprises a scFv formed by at least a VH chain and a VL chain specific to a cell surface marker antigen and one mAb-specific epitope to be bound by a epitope-specific mAb, comprises:
(a) Providing an immune cell;
(b) Introducing into said cell at least one polynucleotide encoding the said chimeric antigen receptor; and,
(c) Expressing said polynucleotide into said cell.

CARs and immune cells comprising them have been extensively disclosed and can be prepared by the skilled person according to known methods. For example, methodologies to prepare CAR and cells comprising such CARs are disclosed earlier. Immune cells comprising a CAR can be prepared by the skilled person according to the methodologies disclosed in the above mentioned references. In a preferred embodiment, immune cells comprising a CAR can be prepared by the skilled person according to the methodologies disclosed in WO2013/176915.

In some embodiments, the immune cell can be derived from an inflammatory T-lymphocyte, a cytotoxic T-lymphocyte, a regulatory T-lymphocyte, or a helper T-lymphocyte.

In some embodiments, immune cell is obtained from a healthy donor. In some embodiments, the immune cell is obtained from a patient.

In some embodiments, the method to engineer cell of the invention further comprises one or more additional genomic modification step. By additional genomic modification step, can be intended the introduction into cells to engineer of one or more protein of interest. Said protein of interest can be a CAR.

In some embodiments, the method of engineering T-cells of invention can comprise:
(a) modifying T-cells by inactivating at least:
   a first gene expressing a target for an immunosuppressive agent, and
   a second gene encoding a component of the T-cell receptor (TCR)
(b) expanding said cells, optionally in presence of said immunosuppressive agent.

An immunosuppressive agent is an agent that suppresses immune function by one of several mechanisms of action. In other words, an immunosuppressive agent is a role played by a compound which is exhibited by a capability to diminish the extent and/or voracity of an immune response. As non-limiting example, an immunosuppressive agent can be a calcineurin inhibitor, a target of rapamycin, an interleukin-2 u-chain blocker, an inhibitor of inosine monophosphate dehydrogenase, an inhibitor of dihydrofolic acid reductase, a corticosteroid or an immunosuppressive antimetabolite.

In a particular embodiment, the genetic modification step of the method relies on the inactivation of one gene selected from the group consisting of CD52, GR, TCR alpha and TCR beta. In another embodiment, the genetic modification step of the method relies on the inactivation of two genes selected from the group consisting of CD52 and GR, CD52 and TCR alpha, CDR52 and TCR beta, GR and TCR alpha, GR and TCR beta, TCR alpha and TCR beta. In another embodiment, the genetic modification step of the method relies on the inactivation of more than two genes. The genetic modification is preferably operated ex-vivo.

The rare-cutting endonucleases used for inactivating the genes in T-cells are preferably Transcription Activator like Effector (TALE), but may be also a Cas9 coupled to a RNA guide as respectively described in WO 2013/176915 and WO 2014/191128.

### Delivery Methods

The different methods described above involve expressing CAR at the surface of a cell. As non-limiting example, said CAR can be expressed by introducing the latter into a cell. CARs can be introduced as transgene encoded by one plasmid vector. Said plasmid vector can also contain a selection marker which provides for identification and/or selection of cells which received said vector.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into cells are known in the art and including as non-limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or virus, in view of being expressed in cells.

### Polynucleotides and Vectors

In one embodiment, said isolated cell according to the present invention comprises a polynucleotide encoding the chimeric antigen receptor carrying the chimeric scFv.

The present invention also relates to polynucleotides, vectors encoding the above described CAR according to the invention.

The polynucleotide may consist in an expression cassette or expression vector (e.g. a plasmid for introduction into a bacterial host cell, or a viral vector such as a baculovirus vector for transfection of an insect host cell, or a plasmid or viral vector such as a lentivirus for transfection of a mammalian host cell).

Those skilled in the art will recognize that, in view of the degeneracy of the genetic code, considerable sequence variation is possible among these polynucleotide molecules. Preferably, the nucleic acid sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

### Therapeutic Applications

In another embodiment, isolated cell or immune cell expressing a CAR as described herein obtained by the different methods or cell line derived from said isolated cell as previously described can be used as a medicament.

In another embodiment, said medicament can be used for treating pathologies such as cancer in a patient in need thereof.

In another embodiment, said isolated cell or immune cell expressing a CAR as described herein I according to the invention or cell line derived from said isolated cell can be used in the manufacture of a medicament for treatment of a pathology such as a cancer in a patient in need thereof.

In another aspect, the present invention relies on methods for treating patients in need thereof, said method comprising at least one of the following steps:
(a) providing an immune-cell obtainable by any one of the methods previously described;
(b) Administrating said transformed immune cells to said patient,

In one embodiment, said immune cell, preferably T cells, of the invention can undergo robust in vivo T cell expansion and can persist for an extended amount of time.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

Said treatment can be used to treat patients diagnosed with cancer, viral infection, autoimmune disorders or Graft versus Host Disease (GvHD). Cancers that may be treated include tumors that are not vascularized, or not yet substantially vascularized, as well as vascularized tumors. The cancers may comprise non solid tumors (such as hematological tumors, for example, leukemias and lymphomas) or may comprise solid tumors. Types of cancers to be treated with the CAR of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

It can be a treatment in combination with one or more therapies against cancer selected from the group of antibodies therapy, chemotherapy, cytokines therapy, dendritic cell therapy, gene therapy, hormone therapy, laser light therapy and radiation therapy.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or nataliziimab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991, Immunology 73(3):316-21; Liu, Albers et al. 1992, 31(16):3896-901; Bierer, Hollander et al. 1993, Curr Opin Immunol 5(5):763-73). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

### Other Definitions

Amino acid residues in a polypeptide sequence are designated herein according to the one-letter code, in which, for example, Q means Gln or Glutamine residue, R means Arg or Arginine residue and D means Asp or Aspartic acid residue.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a ort, v represents g, a or c, b represents g, t or c, h represents a, t or c, and n represents g, a, t or C.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
By chimeric antigen receptor (CAR) is intended molecules that combine a binding domain against a component present on the target cell, for example an antibody-based specificity for a desired antigen (e.g., tumor antigen) with a T cell receptor-activating intracellular domain to generate a chimeric protein that exhibits a specific anti-target cellular immune activity. Generally, CAR consists of an extracellular single chain antibody (scFv) fused to the intracellular signaling domain of the T cell antigen receptor complex zeta chain (scFv:ζ) and have the ability, when expressed in T cells, to redirect antigen recognition based on the monoclonal antibody's specificity.
By “delivery vector” or “delivery vectors” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” are also intended delivery methods to perform transfection.
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g. adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as nonlimiting example, that are able to integrate the genome of a target cell. At the opposite by “non-integrative lentiviral vectors (or NILV)” is meant efficient gene delivery vectors that do not integrate the genome of a target cell through the action of the virus integrase.
Delivery vectors and vectors can be associated or combined with any cellular permeabilization techniques such as sonoporation or electroporation or derivatives of these techniques.
by “mutation” is intended the substitution, deletion, insertion of up to one, two, three, four, five, six, seven, eight, nine, ten, eleven, twelve, thirteen, fourteen, fifteen, twenty, twenty five, thirty, fourty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “functional variant” is intended a catalytically active mutant of a protein or a protein domain; such mutant may have the same activity compared to its parent protein or protein domain or additional properties, or higher or lower activity.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.
The term “subject” or “patient” as used herein includes all members of the animal kingdom including non-human primates and humans. In some embodiments, the patient is human.

In addition to the preceding features, the invention comprises further features which will emerge from the following examples illustrating the method of in vitro sorting or in vivo depleting immune cells expressing CAR for immunotherapy, as well as the appended drawings.

### Example 1. Generation of Rituximab-Driven Depletion Systems Embedded in an Anti-CD123 CAR

All 10 CARs having different conformations in terms of chimeric scFv (anti-CD123 scFv with CD20 mimotope(s)) are depicted in FIG. 4: their resulting polypeptide sequences are shown in SEQ ID NO 1 to 10.

The DNA construct of the 10 CARs are transcribed into their corresponding mRNA via in vitro transcription and used to transfect by electroporation primary T cells freshly isolated from buffy coat via a standard ficoll procedure. One day post transfection, T cells were recovered and used to performed a flow based cytotoxicity assay as described as follows.

### Generation of Anti CD123 CAR T Cells.

To generate primary T cells expressing anti-CD123 CAR, primary T cells are first purified from buffy-coat samples and activated using Dynabeads human T activator CD3/CD28. 3 days post activation, 1 million of activated T cells are transduced with lentiviral vectors harboring an anti-CD123 CAR expression cassette under the control of the Ef1α promoter, at the multiplicity of infection (MOI) of 1. T cells are kept in culture at 37° C. in the presence of 5% CO₂, 20 ng/ml IL-2 (final concentration) and 5% human AB serum in X-vivo-15 media (Lonza) for further characterization. 5 days post transduction, cells are used to perform the flow-based cytotoxicity assay.

### Flow-Based Cytotoxicity Assay

The cytolytic activity and specificity of anti-CD123 CAR T cell are assessed according to the flow cytometry-based cytotoxicity assay as routinely performed (see for example Valton. et Al (2015) Mol Ther; 23(9):1507-1518). This assay consists of labeling 104 CD123 positive tumor cells and 104 CD123-negative control cells with 0.5 mM CellTrace™ CFSE and 0.5 mM CellTrace™ violet (Life Technology) and co-incubating them with 105 effector CAR T cells (E/T ratio of 10:1) in a final volume of 100 μl X-Vivo-15 media, for 5 H at 37° C. Cells are then recovered and labeled with eFluor780 viability marker before being fixed by 4% PFA as described above. Fixed cells are then analysed by flow cytometry to determine their viability. The frequency of specific cell lysis is calculated and displayed in the following:

Frequency of specific cell lysis=(Via CD123+cells with T/CD123−cells with T)/(Via CD123+cells/Via CD123−cells)

where Via CD123+ with T and Via CD123− with T correspond respectively to the % of viable CD123+ cells and CD123− cells obtained after 5 H in the presence of CAR T cells and where Via CD123+ cells and Via CD123− cells correspond respectively to the % of CD123+ cells and CD123− cells obtained after 5 H in the absence of CAR T cells.

The results show that T cells transfected with engineered anti-CD123 CAR are able to kill CD123− positive tumor cell models. As shown in FIG. 5, results from flow-based cytotoxicity assays described above showed that T cells expressing SEQ ID 1-4 displayed the same activity than T cells expressing unmodified anti-CD123 CAR SEQ ID 142 (FIG. 5). These data suggest that insertion of CD20 mimotope in the sequence of the anti-CD123 CAR (SEQ ID 142) does not significantly impair its ability to specifically recognize the CD123 antigen and to kill CD123-expressing tumor cells. In some embodiments, the CARs of the invention comprising one of two mAb-specific epitope, preferably of SEQ ID NO 35 are able to specifically recognize the antigen targeted by the CAR and to kill cells expressing said antigen.

Consistent with these findings, transfected CAR T cells are tested for their capacity to degranulate when exposed to a CD123 recombinant protein coated on a 96 well plate. Together, our experiments are designed to show that insertion of CD20 mimotope in the sequence of the anti-CD123 CAR does not significantly impair its ability to specifically recognize the CD123 antigen.

To demonstrate the ability of rituximab to inhibit T cell cytotoxicity functions through specific recognition of CD20 mimotopes, transfected T cells are incubated in the presence of CD123-positive tumor cells, in the presence or in the absence of rituximab and baby rabbit-complement. The objective is to show that the cytotoxic activity and degranulation capacity of transfected T cells are impaired in the presence of rituximab and baby rabbit complement, indicating further that efficient recognition of engineered anti-CD123 CAR by rituximab leads to T cell depletion.

### Example 2. Flexibility of the mAb-Driven Depletion Systems in the Anti-CD123 Chimeric Antigen Receptor

To further demonstrate the flexibility of the mAb-driven depletion system, different epitopes or mimotopes (SEQ ID NO 35-42) specific for cetuximab, palivizumab and nivolumab mAbs are inserted within the anti-CD123 CAR constructions using the same procedure and architecture as the one used for the CD20 mimotope described in Example 1. The results aim to show that transfected T cells retain their cytolytic activity and degranulation capacity toward CD123 positive tumor cells. In addition, the experiments are designed also to indicate that transfected T cells are depleted by some of the aforementioned mAbs

### Example 3. Rituximab-Dependent Depletion of Anti-CD123 CAR Containing an mAb-Driven Depletion System

To explore the ability of the mAb-driven depletion system to allow depletion of anti-CD123 CAR T cells, transduced T cell expressing a CAR of SEQ ID NO 1, 2, 3 or 4 or an unmodified anti-CD123 CAR (SEQ ID NO 142), were subjected to a complement dependant cytotoxicity assay (CDC).

### CDC Assay

The CDC assay consisted in incubating 0.2 10⁶ transduced T cells either alone, or in the presence of Rituximab (RTX, ROCHE, 400 ng) and Babby Rabbit Complement (BRC, AbD Serotec, ref #C12CA, 100 μL of the solution diluted according to the manufacturer protocol) for 3 hours at 37° C. in a final volume of 400 μL of Xvivo 10% FBS. At the end of incubation, anti CD123-CAR T cells were recovered and labeled with recombinant CD123 protein fused to an FC fragment (SEQ ID 143) and a PE labeled anti-FC secondary monoclonal antibody (Jackson ImmunoResearch, ref #115-115-164, diluted 1/200). Cells were then recovered in PFA 4% before being analyzed by flow cytometry. The flow cytometry gating strategy consisted of determining the viability of anti-CD123 CAR positive T cells (PE positive cells) among the singlet found in the total population of cells. This analysis was performed on cells incubated alone and in the presence of RTX and BRC. Results are expressed as the ratio named “Relative frequency of viable cells among anti-CD123 CAR positive T cells (with respect to control experiment)” described below:

(Frequency of viable cells among anti-CD123 CAR positive T cells obtained in the presence of RTX and BRC)×100/(Frequency of viable cells among anti-CD123 CAR positive T cells obtained in the absence of RTX and BRC)

The results showed that all CAR architectures allowed the RTX-dependent depletion of CAR T cells (FIG. 6). CAR T cells expressing SEQ ID NO 3 and 4 were more efficiently depleted than the ones expressing SEQ ID NO 1 and 2 suggesting that the number of CD20 mimotope present in the CAR architecture influenced the extent and/or the kinetic of T cells depletion.

In some embodiments, CARs of the invention having architectures illustrated by FIG. 4 allow rituximab dependent depletion of CAR T cells. In some embodiments, CAR of the invention comprising at least 2 mAb-specific epitopes, preferably having CAR architecture of SEQ ID NO 3 or 4 are particularly efficiently depleted.

### Example 4. Efficiency of the mAb-Driven Depletion System in Cells Expressing an Anti-BCMA CAR Comprising One or More mAbs Specific Epitopes in the Extracellular Domain

To explore the ability of the mAb-driven depletion system to allow depletion of anti-BCMA CAR T cells, 15 different CAR architectures (SEQ ID 125-139, FIG. 7) were constructed. These architectures were designed to contain 1, 2 or 3 CD20 mimotopes localized at different portions of the extracellular domain of the anti-BCMA CAR (SEQ ID NO 125), i.e in the N terminal domain, in the linker domain separating the V1 and V2 of the ScFv or upstream to the CD8 hinge linking the ScFv to the transmembrane domain of the CAR.

To generate primary T cells expressing anti-BCMA CAR, primary T cells were first purified from buffy-coat samples and activated using Dynabeads human T activator CD3/CD28. 3 days post activation, 5 million of activated T cells were transfected with either 15 or 30 μg of poly adenylated mRNA encoding the different anti-BCMA CAR architectures (SEQ ID 125-139, FIG. 7). T cells were then kept in culture at 37° C. in the presence of 5% CO₂, 20 ng/ml IL-2 (final concentration) and 5% human AB serum in X-vivo-15 media (Lonza) for further characterization. One day post transfection, cells were used to perform the CDC assay, a flow based cytotoxicity assay, a detection assay and an Interferon γ (IFN γ) release assay.

### CDC Assay

The CDC assay consisted in incubating 0.2 106 transfected cells either alone, or in the presence of Rituximab (RTX, ROCHE, 400 ng) and Babby Rabbit Complement (BRC, AbD Serotec, ref #C12CA, 100 μL of the solution diluted according to the manufacturer protocol) for 2 hours at 37° C. in a final volume of 400 μL of Xvivo 10% FBS. At the end of incubation, anti BCMA-CAR T cells were recovered and labeled with recombinant BCMA protein fused to an FC fragment (SEQ ID NO 151) and a PE labeled anti-FC secondary monoclonal antibody (Jackson ImmunoResearch, ref #115-115-164, diluted 1/200). Cells were then recovered in PFA 4% before being analyzed by flow cytometry. The flow cytometry gating strategy was to determine the viability of anti-BCMA CAR positive T cells (PE positive cells) among the singlet found in the total population of cells. This analysis was performed on cells incubated alone and in the presence of RTX and BRC. Results are expressed as the ratio named “Relative frequency of viable cells among BCMA CAR positive T cells (with respect to control experiment)” described below:

(Frequency of viable cells among anti-BCMA CAR positive T cells obtained in the presence of RTX and BRC)×100/(Frequency of viable cells among anti-BCMA CAR positive T cells obtained in the absence of RTX and BRC)

### Flow-Based Cytotoxicity Assay

The cytolytic activity and specificity of anti-BCMA CAR T cell were assessed according to the flow cytometry-based cytotoxicity assay reported in Valton. et Al (2015) Mol Ther; 23(9): 1507-1518. This assay consisted of labeling BCMA positive tumor target cell (T, H929) with 0.5 mM CellTrace™ CFSE (Life Technology, incubation 10 min 37° C. according to manufacturer protocol) and co-incubate them with 105 anti BCMA CAR T effector (E) cells (E/T ratio of 10:1) in a final volume of 100 μl X-Vivo-15 media, for 5 H at 37° C. Cells were then recovered and labeled with eFluor780 viability marker before being fixated by 4% PFA. Fixated cells were then analysed by flow cytometry to determine their viability.

### IFN γ Release Assay

To investigate autoactivation of T cell expressing various anti-BCMA CAR comprising RTX specific epitopes by clinically relevant dose of RTX, primary T cells transfected with mRNA encoding SEQ ID 125, 130-139 were incubated, one day post transfection, for 72 hours in X-vivo-15 medium supplemented with 5% AB serum, 20 ng/ml IL2 in the absence or in the presence of 500 μg/mL RTX at a concentration of 0.1 10⁶ cells/wells in a final volume of 100 μl. CAR T cells were then spun down, the supernatant was recovered and analysed by ELISA (using the Human IFN-gamma Quantikine ELISA Kit, Rand D systems, ref #DIF50) to determine the amount of IFN γ released in the culture media. As positive control for CAR T cell activation and IFN γ release, cells were incubated with 10 μg/mL phytohemagglutinin (PHA).

### Purification of Anti-BCMA CAR T Cells Using Miltenyi CD34 Purification Kit

To test the capacity of certain anti-BCMA CAR architectures (containing the CD34 epitope, SEQ ID NO 144 recognized by the QBEND10 antibody) to be purified, 100 10⁶ primary T cells steadily expressing SEQ ID 128 were purified using the CD34 MicroBead Kit (Miltenyi, ref #130-046-702) according to the manufacturer protocol.

### Results

### Depletability of Anti-BCMA CAR Positive T Cells

The results showed that T cells expressing SEQ ID 126-139 were all depleted to different extents by BRC and RTX in contrast to the unmodified anti-BCMA CAR (SEQ ID NO 125) that was not markedly depleted (FIG. 8A). The results show that efficiency of depletion increase along with the number of CD20 mimotopes present in the CAR architecture. In addition, the results show that separating multiple CD20 mimotopes by domain larger than GS linkers, increased the efficiency of depletion as seen when comparing the extents of depletion obtained with T cells expressing SEQ ID NO 127 and SEQ ID NO 137 containing 3 CD20 mimotopes, as well as SEQ ID NO 139 and SEQ ID NO 136 containing 2 CD20 mimotopes (FIG. 7-8A).

In some embodiments, the CAR of the invention having CAR architecture of SEQ ID NO126-139 allow rituximab dependent depletion of CAR T cell. In some embodiments the CAR of the invention having CAR architecture such as in SEQ ID, 136, 137, 138, i.e where the CAR comprises at least two identical mAb specific epitope separated by one or more other domains (such as VH, VL, VH-L1-VL . . . ) are particularly efficiently depleted.

### Cytotoxic Activity of Anti-BCMA CAR+ T Cells

The flow-based cytotoxicity assay results indicated that all architectures (SEQ ID 126-139) were able to recognize and kill BCMA-expressing H929 tumor cells to a similar extent than T cells expressing the unmodified version of anti-BCMA CAR architecture (SEQ ID NO 125, FIG. 9). Consistent with the results obtained in Example 1, the presence of 1, 2 or 3 mAb specific epitopes and in particular 1, 2 or 3 CD20 mimotopes inside the CAR architecture, did not significantly influence the cytolytic activity of anti-BCMA CAR T cells.

In some embodiments, the CARs of the invention having CAR architectures of SEQ ID NO126-139 have similar cytotoxic activity as compared to a CAR without mAb-specific epitope such as a CAR of SEQ ID 125.

Anti-BCMA Positive CAR T Cells Sorting from an Heterogeneous Population of Cells

To test the capacity of T-cell expressing the anti-BCMA CAR of SEQ ID 128 (FIG. 7A) to be purified from an heterogeneous population of cells, a bulk population of 100×10⁶ primary T cells containing 31.5% of CAR positive cells was purified using the CD34 MicroBead Kit according to the manufacturer protocol. The results showed that the purified fraction harbored about 90% of anti-BCMA CAR positive T cells indicating that the purification process was efficient (FIG. 10). Out of 31.5×10⁶ anti-BCMA CAR positive T cells, about 20×10⁶ anti-BCMA CAR positive T cells were recovered after purification indicating that less than 40% of anti-BCMA CAR positive T cells were lost throughout the purification process.

### IFN γ Release Assay

The ELISA assay results showed that the presence of one or multiple CD20 mimotopes within the CAR architecture did not influence the propensity of CAR T cells to be activated by RTX (FIG. 11). Indeed The results showed that the level of IFN γ released by all architectures in the presence of RTX were similar to the basal level of IFN γ released in the absence of RTX.

### Example 5. Hybrid Anti-BCMA Chimeric Antigen Receptor Architectures for Optimal Depletion and Purification of CAR T Cells

To improve the depletability of anti-BCMA CAR T cells and at the same time, allow to sort them, two new hybrid CAR architectures SEQ ID NO 140 and 141 (FIG. 7C) were designed. These two architectures contained three CD20 mimotopes separated from one another by protein domains and one CD34 epitope. Their ability to be depleted by RTX and BRC was assessed by CDC assay according to the protocol described in Example 4. The results showed that these two architectures were efficiently depleted to a similar extent than T cells expressing SEQ ID 137 (FIG. 8B). Their cytolytic properties were also assessed using the flow-based assay described earlier. The results showed that they share similar cytotoxic activity than the T cells expressing the unmodified anti-BCMA CAR T cells (SEQ ID NO 125) indicating that the presence of CD20 mimotopes and CD34 epitope (SEQ ID NO 35 and 144 respectively) did not negatively impact the cytolytic activity of CAR T cells.

In some embodiments the CAR of the invention having CAR architecture such as in SEQ ID 140, 141, i.e where the CAR comprises at three identical mAb-specific epitope recognized by an approved antibody such as rituximab which can be used for depletion of the cells and one mAb specific eptitope which can be used for purification are particularly efficiently depleted and can also be efficiently purified.

Additional CARs based on the architecture of SEQ ID NO 140 and 141 but comprising VH and VL of ScFv specific for CD19 (CAR of SEQ ID NO 162-163 and 168-169), CD123 (CAR of SEQ ID NO 164-165), CD20 (CAR of SEQ ID NO 166-167) were assembled according to the protocol described in Example 4. Their ability to be depleted by RTX and BRC can be assessed by CDC assay according to the protocol described in Example 4.

### Example 6. Universal Detection of CAR T Cells Bearing a mAb-Driven Depletion System

Monitoring and comparing proliferation of different CAR T cells in vivo has been tiedous and cumbersome due to the lack of universal detection system. The ability of different CAR architectures to be detected was tested by flow cytometry using RTX as primary antibody and an FITC-coupled anti-Fab′2 monoclonal antibody (Life technologies, ref #H10101C, diluted 1/200) or using an APC labeled anti-CD34 monoclonal antibody named QBEND10 (Miltenyi Biotec, ref #130-090-954, diluted 1/25). Results were compared side by side with detection performed with recombinant BCMA protein fused to an FC fragment (SEQ ID NO 151) and a PE labeled anti-FC secondary monoclonal antibody (Jackson ImmunoResearch, ref #115-115-164, diluted 1/200).

The results, showed that the frequency of positive CAR T cells expressing SEQ ID NO 128 and 130-139 detected with RTX were similar to the ones obtained when they were detected with recombinant BCMA protein (FIG. 12). Similar results were found when CAR T cells expressing SEQ ID NO 128 were detected with QBEND10 and rituximab (FIG. 13). Altogether, The results showed that the presence of CD20 mimotope or CD34 epitope allow for efficient and universal detection of different CAR architectures.

### Example 7—Anti-BCMA CAR T Cells Expressing Anti BCMA CAR Comprising One, Two or Three mAB Specific Epitopes

### 7.1—Plasmids

The below CD20 mimotope-containing CARs are codon-optimized, synthesized and subcloned into the lentiviral vector pLVX-EF1a-IRES-Puro (Clontech) using the EcoRI (5′) and MIuI (3′) restriction sites (thus removing the IRES-Puro cassette). Lentiviruses are produced using psPAX2, an HIV-1 gag-pol packaging plasmid, and pMD2.G, a VSV-G expression plasmid.

### BC30 (SEQ ID NO 145) Comprises the Following Domains:

leader-BCMA30 VH-linker-BCMA30 VL-CD8 Hinge-CD8 TM-4-1BB-CD3z wherein BCMA30 VH and BCMA30 VL are respectively SEQ ID NO 97 and SEQ ID NO 98.

### BC30-LM (SEQ ID NO 146) Comprises the Following Domains:

Leader-BCMA30 VH-linker-BCMA30 VL-linker(L)-Mimotope (M)-CD8 Hinge-CD8 TM-4-1BB-CD3z wherein BCMA30 VH and BCMA30 VL are respectively SEQ ID NO 97 and SEQ ID NO 98 and the mimotope is SEQ ID NO35.

### BC30-LML (SEQ ID NO 147) Comprises the Following Domains:

Leader-BCMA30 VH-linker-BCMA30 VL-linker(L)-Mimotope (M)-linker(L)-CD8 Hinge-CD8 TM-4-1BB-CD3z wherein BCMA30 VH and BCMA30 VL are respectively SEQ ID NO 97 and SEQ ID NO 98 and the mimotope is SEQ ID NO35.

### BC30-LMLM (SEQ ID NO 148) Comprises the Following Domains:

Leader-BCMA30 VH-linker-BCMA30 VL-linker(L)-Mimotope (M)-linker(L)-Mimotope (M)-CD8 Hinge-CD8 TM-4-1BB-CD3z wherein BCMA30 VH and BCMA30 VL are respectively SEQ ID NO 97 and SEQ ID NO 98 and the mimotopes are both SEQ ID NO 35.

### BC30-LMLML (SEQ ID NO 149) Comprises the Following Domains:

Leader-BCMA30 VH-linker-BCMA30 VL-linker(L)-Mimotope (M)-linker(L)-Mimotope (M)-linker(L)-CD8 Hinge-CD8 TM-4-1BB-CD3z wherein BCMA30 VH and BCMA30 VL are respectively SEQ ID NO 97 and SEQ ID NO 98 and the mimotopes are both SEQ ID NO 35.

### 7.2—T Cell Activation and Lentiviral Transduction

Untouched T cells are isolated from human peripheral blood mononuclear cells (PBMCs) using the Pan T Cell isolation kit (Miltenyi Biotec) and activated for three days with antibodies against human CD2, CD3, and CD28 (T Cell activation/expansion kit—Miltenyi Biotec). Lentiviral vectors (LV) are produced by transient transfection of sub-confluent HEK-293T/17 (American Type Culture Collection (ATCC)) cells in 6-well plates. Briefly, pLVX, psPAX2, and pMD2.G plasmids are transfected at a 4:3:1 ratio, respectively, using Lipofectamine 2000 (Invitrogen) following the manufacturer's instructions. The following day, the media is replaced with T cell culture medium (5% human AB serum in X-vivo-15 medium (Lonza)), and 48 h after transfection the LV supernatant is harvested and filtered through a 0.45 μm syringe filter (Millipore). Activated T cells are seeded at 0.25×10⁶ cells/mL in T cell culture medium containing 40 ng/ml IL-2 and transduced by adding an equal volume of fresh LV supernatant. Cells are cultured at 37° C. and 5% CO2 for three days and used for flow cytometry analysis or expanded in fresh T cell medium containing 20 ng/ml IL-2.

### 7.3—Detection of BCMA CARs Containing CD20 Mimotopes by Flow Cytometry

To test the utility of intra CAR CD20 mimotopes for detection and tracking of CAR-T cells, flow cytometry analysis is performed on transduced T cells using either biotinylated-BCMA protein, which binds the scFV region of the CAR followed by PE-conjugated streptavidin, or the anti-CD20 antibody rituximab followed by FITC-conjugated anti-human IgG (Rituximab (FITC)). FIGS. 14A and 14B show that T cells transduced with the different CD20-mimotope-containing CARs are detected with comparable efficiency by flow cytometry using biotinylated-BCMA followed by PE-conjugated streptavidin. Detection of intra CAR CD20 mimotope(s) with rituximab is weak in cells transduced with the LM construct (15.5%) but very high in all other formats tested. For example, the LMLML CAR is detected in 85.6% of the cells, indicating that this format allows the identification of virtually all cells expressing the CAR (FIGS. 14A and 14B). Thus, the presence of two CD20 epitopes separated by flexible linkers allows for enhanced binding to rituximab and provides an optimum system to detect CAR+ cells.

The functionality of intra CAR CD20 epitopes for CAR-T cell detection is assessed by comparison with the RQR8 marker/suicide gene system (SEQ ID NO 150), which consists of a compact protein containing two CD20 epitopes and a CD34 epitope that is normally co-expressed with the CAR (Philip, Blood 2014). For this experiment, T cells are transduced with a lentivirus that allows the co-expression of the BCMA30 CAR (SEQ ID NO 145) and the RQR8 protein (SEQ ID NO 150) (BC30-RQR8 construct). For comparison, T cells are transduced with the BCMA30 LMLML CAR construct (BC30-R2 construct—SEQ ID NO 149) and analyzed by flow cytometry three days post-transduction. In addition, non-transduced (NT) T cells serve as negative control. FIG. 15 aims to show that incorporating the CD20 epitopes in the CAR molecule significantly improves detection of CAR-T cells with the anti-CD20 antibody rituximab. In addition, increased transduction efficiency and CAR expression is observed in cells transduced with the BC30-R2 construct compared with those transduced with the vector encoding RQR8 and the CAR, as indicated by flow cytometry analysis with biotinylated BCMA (FIG. 15). Thus, insertion of CD20 epitopes into the CAR molecule enables enhanced transduction, improved detection, and absolute correlation between CAR expression and mAb specific epitope(s) expression.

### 7.4—Intra CAR CD20 Epitopes Sensitize CAR-T Cells to Complement-Dependent Cytotoxicity

The ability of intra CAR CD20 epitopes to enable selective elimination of CAR-T cells is evaluated in vitro using a CDC assay. The objective is to show that the presence of CD20 epitopes in the CAR molecule renders CAR-T cells highly susceptible to rituximab-mediated depletion. For this experiment, T cells transduced with either the BC30-R2 construct or the BC30-RQR8 construct are mixed with 25% baby-rabbit complement (AbD serotec) in the presence or absence of rituximab (100 μg/mL) and incubated at 37° C. and 5% CO2 for 4 hours. Selective deletion of CAR-T cells is determined by flow cytometry analysis using biotinylated BCMA protein. FIG. 16 shows that while both the RQR8 and intra CAR CD20 epitope suicide gene systems enable CAR-T cell depletion, cells transduced with the BC30-R2 construct are depleted more efficiently than those expressing RQR8. As expected, T cells expressing the BCMA30 CAR but no CD20 epitopes (BC30 construct) are spared. These differences may be due to the high expression of the BC30-R2 CAR and the absolute correlation between CAR expression and suicide gene expression.

7.5—Incorporation of CD20 Epitopes into CARs does not Impair the Cytolytic Activity of CAR-T Cells

The possibility that insertion of CD20 epitopes between the hinge and the scFv regions of the CAR might impair CAR activity is evaluated in a cytotoxicity assay. Briefly, T cells expressing either the BC30-R2 construct or the BC30-RQR8 construct are incubated with Luciferase-positive MM1S target cells at different ratios. For these killing assays, cells are seeded in 96-well white opaque tissue culture plates in a final volume of 100 μl of 5% human AB serum in X-vivo-15 medium (Lonza). After 4 hours, cells are equilibrated to room temperature and one volume of Bright-Glo™ Reagent (Promega) is added to each well. Luminescence is measured in a GLOMAX 96 microplate luminometer (Promega) and percentage of cell lysis is calculated according to the following formula:

100×(1—(Sample lysis−max lysis)/(Spontaneous lysis−max lysis)). Maximum lysis is determined by addition of 8% Triton X-100 (Sigma) to Luc+ MM1S cells. For spontaneous lysis, MM1S cells are incubated in the absence of effector CAR-T cells.

The results show that BC30-R2 CAR-T cells effectively eliminate BCMA-expressing MM1S cells in vitro (FIG. 17). Moreover, the cytolytic activity of BC30-R2 CAR-T cells is not influenced by rituximab (100 μg/mL), which is added to the effector cell population 2 h before these cells are mixed with the target cells (FIG. 17). This experiment aims to demonstrate that insertion of CD20 epitopes into the BC30 CAR molecule does not affect its ability to mediate killing of BCMA+ target cells, even in the presence of rituximab.

7.6 Rituximab Binding to Intra CAR CD20 Epitopes does not Lead to CAR-T Cell Activation

To investigate if crosslinking of CARs by rituximab might lead to T cell activation due to CAR aggregation on the cell surface, BC30-R2 CAR-T cells are grown in the presence of rituximab. The anti-CD3 OKT3 antibody (eBioscience) causes crosslinking of the T cell receptor (TCR) resulting in cellular activation and proliferation and is used as a positive control. Briefly, BC30-R2 CAR-T cells are cultured in T cell medium in the presence/absence of rituximab for three days. T cell activation is then assessed by measuring the expression of the activation markers CD25 and CD69 using flow cytometry. This experiment shows that the percentage of activated T cells in the presence of RTX is not significantly different from the control (PBS). Therefore, soluble rituximab has no significant effect on the activation state of BC30-R2 CAR-T cells (FIG. 18).

### REFERENCES

Arbiza J., Taylor G., López J. A., Furze J., Wyld S., Whyte P., Stott E. J., Wertz G., Sullender W., Trudel M., et al. (1992), Characterization of two antigenic sites recognized by neutralizing monoclonal antibodies directed against the fusion glycoprotein of human respiratory syncytial virus. J Gen Virol.; 73 (9):2225-34).
Benjamin, R J and Waldmann, H. (1986). Induction of tolerance by monoclonal antibody therapy. Nature 520: 449-451.
Boch, J., H. Scholze, et al. (2009). Breaking the code of DNA binding specificity of TAL-type III effectors. Science 326(5959): 1509-12.
Budde, L. E., Berger C, Lin Y, Wang J, Lin X, Frayo S E, Brouns S A, Spencer D M, Till B G, Jensen M C, Riddell S R (2013). Combining a CD20 chimeric antigen receptor and an inducible caspase 9 suicide switch to improve the efficacy and safety of T cell adoptive immunotherapy for lymphoma. PLOS One. December 17; 8(12):e82742
Buller R. M., Holmes K. L., Hügin A., Fredrickson T. N., Morse H. C. (1987). Induction of cytotoxic T cell responses in vivo in the absence of CD4 helper cells. Nature.; 328:77-79.
Cobbold, S. P., Martin, G., Qin, S., and Waldmann, H. (1986). Monoclonal antibodies to promote marrow engraftment and tissue graft rejection. Nature 323:164-166
Dolan D E, Gupta S. 2014 PD-1 pathway inhibitors: changing the landscape of cancer immunotherapy. Cancer Control. 21(3):231-7.
Epa, V. C., O. Dolezal, et al. (2013). Structural model for the interaction of a designed Ankyrin Repeat Protein with the human epidermal growth factor receptor 2. PLOS One 8(3): e59163.
Fedorov V. D., Themeli M and Sadelain M. (2013). PD-1- and CTLA-4-Based Inhibitory Chimeric Antigen Receptors (iCARs) Divert Off-Target Immunotherapy Responses. Sci Transl Med:5 (215), 215
Friedrich, K., J. R. Hanauer, et al. (2013). DARPin-targeting of measles virus: unique bispecificity, effective oncolysis, and enhanced safety. Mol Ther 21(4): 849-59.
Jena, B., G. Dotti, et al. (2010). Redirecting T-cell specificity by introducing a tumor-specific chimeric antigen receptor. Blood 116(7): 1035-44.
Jost, C., J. Schilling, et al. (2013). Structural Basis for Eliciting a Cytotoxic Effect in HER2-Overexpressing Cancer Cells via Binding to the Extracellular Domain of HER2. Structure 21(11): 1979-91.
Moscou, M. J. and A. J. Bogdanove (2009). A simple cipher governs DNA recognition by TAL effectors. Science 326(5959): 1501.
Park, T. S., S. A. Rosenberg, et al. (2011). Treating cancer with genetically engineered T cells. Trends Biotechnol 29(11): 550-7.
Philip B, Kokalaki E, Mekkaoui L, Thomas S, Straathof K, Flutter B, Marin V, Marafioti T, Chakraverty R, Linch D, Quezada S A, Peggs K S, Pule M (2014). A highly compact epitope-based marker/suicide gene for easier and safer T-cell therapy. Blood. 124(8): 1277-87
Riemer A. B., Kurz H., Klinger, M., Scheiner, O., Zielinski, C., and Jensen-Jarolim, E. (2005), Vaccination with cetuximab mimotopes and biological properties of induced anti-epidermal growth factor receptor antibodies, J Natl Cancer Inst.; 97(22):1663-70)
Valton J., Guyot V., Marechal A., Filhol J M., Juillerat A., Duclert A., Duchateau P., Poirot L. (2015) A multidrug resistant engineered CAR T cell for allogeneic combination immunotherapy. Mol Ther; 23(9):1507-1518
Philip B, Kokalaki E, Mekkaoui L, Thomas S, et al. A highly compact epitope-based marker/suicide gene for easier and safer T-cell therapy. Blood 2014; 124(8): 1277-87.

### Embodiments of the Invention

1. A polypeptide encoding a chimeric antigen receptor (CAR) comprising at least one extracellular binding domain that comprises a scFv formed by at least a VH chain and a VL chain specific to a cell surface marker antigen, wherein said extracellular binding domain includes at least one mAb-specific epitope to be bound by a epitope-specific mAb for in vitro cell sorting and/or in vivo cell depletion of the immune cells expressing said CAR.
2. A polypeptide according to embodiment 1, wherein said mAb-specific epitope is located between the VH and VL chains.
3. A polypeptide according to embodiment 1 or 2, wherein said VH and VL chains, and mAb specific-epitope are bound together by at least one linker and to the transmembrane domain of said CAR by a hinge.
4. A polypeptide according to embodiment 3, wherein the mAb-epitope is joined to the VH and VL chains by two linkers.
5. A polypeptide according to anyone of embodiment 1 to 4, wherein the mAb-epitope is from one polypeptide selected from those listed in Table 1.
6. A polypeptide according to anyone of embodiment 1 to 4, wherein said VH and VL chains have as antigenic target sequence of over 80% identity, preferably over 90%, and more preferably over 95% with SEQ ID NO 43 (CD19 antigen), SEQ ID NO 44 (CD38 antigen), SEQ ID NO 45 (CD123 antigen), SEQ ID NO 46 (CS1 antigen), SEQ ID NO 47 (BCMA antigen), SEQ ID NO 48 (FLT-3 antigen), SEQ ID NO 49 (CD33 antigen), SEQ ID NO 50 (CD70 antigen), SEQ ID NO 51 (EGFR-3v antigen) and SEQ ID NO 52 (WT1 antigen).
7. A polypeptide according to anyone of embodiment 1 to 5, wherein said VH and VL chains have as antigenic target sequence of over 80% identity, preferably over 90%, and more preferably over 95% with SEQ ID NO 53-64 (CD19 antigen), SEQ ID NO 65-76 (CD33 antigen), SEQ ID NO 77-84 (5T4 antigen), SEQ ID NO 85-90 (ROR1 antigen), SEQ ID NO 91-94 (EGFRvIII antigen), SEQ ID NO 95-102 (BCMA antigen), SEQ ID NO 103-112 (CS1 antigen) and SEQ ID NO 113-124 (CD123 antigen).
8. A polypeptide according to anyone of embodiment 1-7, wherein said VH and VL chains have as epitope target sequence of over 80% identity, preferably over 90%, and more preferably over 95% identity with the CD20 antigen of SEQ ID NO.11.
9. A polypeptide according to anyone of embodiment 1-8, wherein the CAR is a single-chain CAR.
10. A polypeptide according to embodiment 9, wherein the said CAR polypeptide shares over 80% identity, preferably over 90%, and more preferably over 95% with SEQ ID NO 1 to 10.
11. A polypeptide according to anyone of embodiment 1-10 wherein the CAR is a multi-chain CAR.
12. A polynucleotide encoding a chimeric antigen receptor according to anyone of embodiments 1 to 9, wherein said CAR comprises a CD3 zeta signaling domain and co-stimulatory domain from 4-1BB.
13. An expression vector comprising a nucleic acid of embodiment 12.
14. An engineered immune cell expressing at its cell surface a chimeric antigen receptor according to anyone of embodiments 1 to 12.
15. An engineered immune cell according to embodiment 14, derived from inflammatory T-lymphocytes, cytotoxic T-lymphocytes, regulatory T-lymphocytes or helper T-lymphocytes.
16. An engineered immune cell according to embodiment 14 or 15 for use as a medicament.
17. A method for engineering an immune cell of anyone of embodiment 14-16, comprising:
(a) Providing an immune cell;
(b) Introducing into said cell at least one polynucleotide encoding the chimeric antigen receptor according to anyone of embodiment 1-12.
(c) Expressing said polynucleotide into said cell.
18. A method for engineering an immune cell of embodiment 17, wherein immune cell is a T-cell.
19. A method for sorting CAR-expressing immune cells comprising contacting a population of immune cells engineered according to anyone of embodiment 14-16 with antigen-specific mAbs in order to collect CAR-expressing immune cells only.
20. A method for sorting CAR-expressing immune cells according to embodiment 19 wherein the mAb is rituximab.
21. A method for sorting CAR-expressing immune cells according to anyone of embodiment 19-20, wherein the immune cell is a T-cell.
22. A method for depleting immune cell engineered according to anyone of embodiment 14-16, or CAR-expressing immune cell sorted according to anyone of embodiment 19-21, comprising contacting said immune cell or said CAR-expressing immune cell with epitope-specific mAbs.
23. A method for depleting immune cell or CAR-expressing immune cell according to embodiment 22, wherein said epitope-specific mAb is conjugated by a molecule able to activate the complement system.
24. A method for depleting immune cell CAR-expressing immune cell according to anyone of embodiment 22-23, wherein a cytotoxic drug is coupled to the epitope-specific mAbs.
25. A method for depleting immune cell CAR-expressing immune cell according to anyone of embodiment 22-24, wherein the mAb-specific antigen is CD20 antigen and the epitope-specific mAb is rituximab.
26. A method for depleting immune cell CAR-expressing immune cell according to anyone of embodiment 22-25, comprising contacting said immune cell or CAR-expressing immune cell with bi-specific mAb (BsAb) able to bind both the mAb-specific epitope borne on said cells and to an surface antigen borne on an effector (and cytotoxic) cell.
27. A method for depleting immune cell CAR-expressing immune cell according to anyone of embodiment 22-26, wherein said immune cell is a T-cell.
28. Method for regulating the activation of an engineered immune cell comprising at least the step of:
(i) Endowing said immune cell with a CAR, which extracellular binding domain comprises a scFv recognizing a cell surface marker linked to a mAb specific epitope
(ii). Expanding said immune cell expressing said CAR and said mAb epitope on its surface
(iii) Contacting the resulting immune cells with the mAb specific to said epitope to immobilize said immune cells.

1-62. (canceled)

63. An in vivo method for eliminating at least one engineered immune cell expressing a chimeric antigen receptor (CAR) polypeptide, the method comprising:
(i) administering a composition comprising at least one antibody specific for an epitope of SEQ ID NO: 35 to a subject in need thereof;
wherein the CAR polypeptide comprises an extracellular binding domain comprising a scFv having a VH chain and a VL chain specific for an antigen and at least two epitopes of SEQ ID NO: 35; wherein at least two of the at least two epitopes of SEQ ID NO: 35 are separated by the VH chain, the VL chain, an epitope of SEQ ID NO: 144, or any combination thereof; and
(ii) eliminating the at least one engineered immune cell from the subject.

64. The method of claim 63, wherein the extracellular binding domain of the CAR polypeptide comprises at least one linker in one or more of the following positions:
between the VH chain and one of the at least two epitopes of SEQ ID NO: 35, or
between the VH chain and two of the at least two epitopes of SEQ ID NO: 35;
between the VL chain and one of the at least two epitopes of SEQ ID NO: 35, or
between the VL chain and two of the at least two epitopes of SEQ ID NO: 35;
and/or
between the epitope of SEQ ID NO: 144 and one of the at least two epitopes of SEQ ID NO: 35, or between the epitope of SEQ ID NO: 144 and two of the at least two epitopes of SEQ ID NO: 35.

65. The method of claim 63, wherein the at least one linker comprises glycine and/or serine.

66. The method of claim 65, wherein the at least one linker comprises one of the following amino acid sequences: SGG, GGS, SGGS (SEQ ID NO: 186), SSGGS (SEQ ID NO: 187), GGGG (SEQ ID NO: 188), SGGGG (SEQ ID NO: 189), GGGGS (SEQ ID NO: 190), SGGGGS (SEQ ID NO: 191), GGGGGS (SEQ ID NO: 192), SGGGGGS (SEQ ID NO: 193), SGGGGG (SEQ ID NO: 194), GSGGGGS (SEQ ID NO: 195), GGGGGGGS (SEQ ID NO: 196), SGGGGGGG (SEQ ID NO: 197), SGGGGGGGS (SEQ ID NO: 198), or SGGGGSGGGGS (SEQ ID NO: 199).

67. The method of claim 63, wherein the extracellular binding domain of the CAR polypeptide comprises a hinge region from CD8 alpha.

68. The method of claim 63, wherein the CAR polypeptide comprises a transmembrane domain comprising a transmembrane region(s) from CD8 alpha.

69. The method of claim 63, wherein the CAR polypeptide comprises an intracellular domain comprising a CD3 zeta signaling domain.

70. The method of claim 63, wherein the CAR polypeptide comprises an intracellular domain comprising a CD3 zeta signaling domain and/or a 4-1BB co-stimulatory domain.

71. The method of claim 63, wherein the CAR polypeptide is a single-chain CAR or a multi-chain CAR.

72. The method of claim 63, wherein the antigen is a tumor-associated surface antigen.

73. The method of claim 63, wherein the antigen is selected from the group consisting of: ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, GD3, C-type lectin-like molecule-1 (CLL-1), ductal-epithelial mucine, gp36, TAG-72, glycosphingolipids, glioma-associated antigen, ß-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelin, 5T4, ROR1, Nkp30, NKG2D, tumor stromal antigens, extra domain A (EDA) of fibronectin, extra domain B (EDB) of fibronectin, A1 domain of tenascin-C (TnC A1), fibroblast associated protein (fap), LRP6, melamona-associated Chondroitin Sulfate Proteoglycan (MCSP), CD38/CS1, MART1, WT1, MUC1, LMP2, Idiotype, NY-ESO-1, Ras mutant, gp100, proteinase 3, bcr-abl, tyrosinase, hTERT, EphA2, ML-TAP, ERG, NA17, PAX3, ALK, Androgen receptor, CD3, CD4, CD8, CD24, CD25, CD33, CD34, CD70, CD79, CD116, CD117, CD135, CD123, CD133, CD138, CTLA-4, B7-1 (CD80), B7-2 (CD86), endoglin, BCMA, and FLT-3.

74. The method of claim 63, wherein the antigen is selected from the group consisting of: an HIV-specific antigen, an EBV-specific antigen, a CMV-specific antigen, an HPV-specific antigen, a Lasse Virus-specific antigen, and an Influenza Virus-specific antigen.

75. The method of claim 63, wherein the antigen comprises an amino acid sequence selected from the group consisting of: SEQ ID: NO 43 (CD19 antigen), SEQ ID NO: 44 (CD38 antigen), SEQ ID NO: 45 (CD123 antigen), SEQ ID NO: 46 (CS1 antigen), SEQ ID NO: 47 (BCMA antigen), SEQ ID NO: 48 (FLT-3 antigen), SEQ ID NO: 49 (CD33 antigen), SEQ ID NO: 50 (CD70 antigen), SEQ ID NO: 51 (EGFR-3v antigen) and SEQ ID NO: 52 (WT1 antigen).

76. The method of claim 63, wherein the at least one antibody specific for an epitope of SEQ ID NO: 35 is rituximab.

77. The method of claim 63, wherein the at least one engineered immune cell is derived from an inflammatory T-lymphocyte, a cytotoxic T-lymphocyte, a regulatory T-lymphocyte, or a helper T-lymphocyte.

78. The method of claim 63, wherein the at least one engineered immune cell is a T-cell.

79. The method of claim 63, wherein the method is carried out after administering to the subject a composition comprising the at least one engineered immune cell expressing the CAR polypeptide.

80. The method of claim 79, wherein the at least one engineered immune cell expressing the CAR has been sorted out ex vivo using an antibody specific for the epitope of SEQ ID NO: 35 or an antibody specific for the epitope of SEQ ID NO: 144 prior to administration to the subject.

81. The method of claim 80, wherein the antibody specific for an epitope of SEQ ID NO: 144 is QBEND-10.

82. The method of claim 63, wherein the composition comprising the at least one antibody specific for an epitope of SEQ ID NO: 35 is administered to a subject via infusion.

83. The method of claim 63, wherein the composition comprising the at least one antibody specific for an epitope of SEQ ID NO: 35 is administered to a subject at least once per week.

84. The method of claim 63, wherein administering the composition comprising the at least one antibody specific for an epitope of SEQ ID NO: 35 to the subject decreases the amount of engineered immune cells present in the subject by at least about 10%.

85. An engineered immune cell expressing a chimeric antigen receptor (CAR) polypeptide, wherein said CAR polypeptide comprises:
(i) an extracellular binding domain comprising a scFv having a VH chain and a VL chain specific for an antigen and at least two epitopes of SEQ ID NO: 35;
wherein at least two of the at least two epitopes of SEQ ID NO: 35 are separated by the VH chain, the VL chain, an epitope of SEQ ID NO: 144, or any combination thereof;
(ii) a hinge region from CD8 alpha;
(iii) a transmembrane domain comprising a transmembrane region(s) from CD8 alpha; and
(iv) an intracellular domain comprising a CD3 zeta signaling domain and/or a 4-1BB co-stimulatory domain.

86. A chimeric antigen receptor (CAR) polypeptide, wherein said CAR polypeptide comprises:
(i) an extracellular binding domain comprising a scFv having a VH chain and a VL chain specific for an antigen and at least two epitopes of SEQ ID NO: 35;
wherein at least two of the at least two epitopes of SEQ ID NO: 35 are separated by the VH chain, the VL chain, an epitope of SEQ ID NO: 144, or any combination thereof;
(ii) a hinge region from CD8 alpha;
(iii) a transmembrane domain comprising a transmembrane region(s) from CD8 alpha; and
(iv) an intracellular domain comprising a CD3 zeta signaling domain and/or a 4-1BB co-stimulatory domain.
